SEQUENCE LISTING (32 sequences)

SEQ ID NO: 1 - DNA, 2625 bp, Homo sapiens
  CDS (252)..(1433)
acttgtcatggcgactgtccagctttgtgccaggagcctcgcaggggttgatgggattggggttttcccctcccatgtgctcaagactggcgctaaaagttttgagcttctcaaaagtctagagccaccgtccagggagcaggtagctgctgggctccggggacactttgcgttcgggctgggagcgtgctttccacgacggtgacacgcttccctggattggcagccagactgccttccgggtcactgccatggaggagccgcagtcagatcctagcgtcgagccccctctgagtcaggaaacattttcagacctatggaaactacttcctgaaaacaacgttctgtcccccttgccgtcccaagcaatggatgatttgatgctgtccccggacgatattgaacaatggttcactgaagacccaggtccagatgaagctcccagaatgccagaggctgctccccccgtggcccctgcaccagcagctcctacaccggcggcccctgcaccagccccctcctggcccctgtcatcttctgtcccttcccagaaaacctaccagggcagctacggtttccgtctgggcttcttgcattctgggacagccaagtctgtgacttgcacgtactcccctgccctcaacaagatgttttgccaactggccaagacctgccctgtgcagctgtgggttgattccacacccccgcccggcacccgcgtccgcgccatggccatctacaagcagtcacagcacatgacggaggttgtgaggcgctgcccccaccatgagcgctgctcagatagcgatggtctggcccctcctcagcatcttatccgagtggaaggaaatttgcgtgtggagtatttggatgacagaaacacttttcgacatagtgtggtggtgccctatgagccgcctgaggttggctctgactgtaccaccatccactacaactacatgtgtaacagttcctgcatgggcggcatgaaccggaggcccatcctcaccatcatcacactggaagactccagtggtaatctactgggacggaacagctttgaggtgcgtgtttgtgcctgtcctgggagagaccggcgcacagaggaagagaatctccgcaagaaaggggagcctcaccacgagctgcccccagggagcactaagcgagcactgcccaacaacaccagctcctctccccagccaaagaagaaaccactggatggagaatatttcacccttcagatccgtgggcgtgagcgcttcgagatgttccgagagctgaatgaggccttggaactcaaggatgcccaggctgggaaggagccaggggggagcagggctcactccagccacctgaagtccaaaaagggtcagtctacctcccgccataaaaaactcatgttcaagacagaagggcctgactcagactgacattctccacttcttgttccccactgacagcctccctcccccatctctccctcccctgccattttgggttttgggtctttgaacccttgcttgcaataggtgtgcgtcagaagcacccaggacttccatttgctttgtcccggggctccactgaacaagttggcctgcactggtgttttgttgtggggaggaggatggggagtaggacataccagcttagattttaaggtttttactgtgagggatgtttgggagatgtaagaaatgttcttgcagttaagggttagtttacaatcagccacattctaggtaggggcccacttcaccgtactaaccagggaagctgtccctcatgttgaattttctctaacttcaaggcccatatctgtgaaatgctggcatttgcacctacctcacagagtgcattgtgagggttaatgaaataatgtacatctggccttgaaaccaccttttattacatggggtctaaaacttgacccccttgagggtgcctgttccctctccctctccctgttggctggtgggttggtagtttctacagttgggcagctggttaggtagagggagttgtcaagtcttgctggcccagccaaaccctgtctgacaacctcttggtccaccttagtacctaaaaggaaatctcaccccatcccacaccctggaggatttcatctcttgtatatgatgatctggatccaccaagacttgttttatgctcagggtcaatttcttttttcttttttttttttttttttctttttctttgagactgggtctcgctttgttgcccaggctggagtggagtggcgtgatcttggcttactgcagcctttgcctccccggctcgagcagtcctgcctcagcctccggagtagctgggaccacaggttcatgccaccatggccagccaacttttgcatgttttgtagagatggggtctcacagtgttgcccaggctggtctcaaactcctgggctcaggcgatccacctgtctcagcctcccagagtgctgggattacaattgtgagccaccacgtccagctggaagggtcaacatcttttacattctgcaagcacatctgcattttcaccccacccttcccctccttctccctttttatatcccatttttatatcgatctcttattttacaataaaactttgctgcca

SEQ ID NO: 2 - protein, 393 aa, Homo sapiens [sequence omitted]

SEQ ID NO: 3 - DNA, 1700 bp, Homo sapiens
  CDS (275)..(895)
cttgcctgcaaacctttacttctgaaatgacttccacggctgggacgggaaccttccacccacagctatgcctctgattggtgaatggtgaaggtgcctgtctaacttttctgtaaaaagaaccagctgcctccaggcagccagccctcaagcatcacttacaggaccagagggacaagacatgactgtgatgaggagctgctttcgccaatttaacaccaagaagaattgaggctgcttgggaggaaggccaggaggaacacgagactgagagatgaattttcaacagaggctgcaaagcctgtggactttagccagacccttctgccctcctttgctggcgacagcctctcaaatgcagatggttgtgctcccttgcctgggttttaccctgcttctctggagccaggtatcaggggcccagggccaagaattccactttgggccctgccaagtgaagggggttgttccccagaaactgtgggaagccttctgggctgtgaaagacactatgcaagctcaggataacatcacgagtgcccggctgctgcagcaggaggttctgcagaacgtctcggatgctgagagctgttaccttgtccacaccctgctggagttctacttgaaaactgttttcaaaaactaccacaatagaacagttgaagtcaggactctgaagtcattctctactctggccaacaactttgttctcatcgtgtcacaactgcaacccagtcaagaaaatgagatgttttccatcagagacagtgcacacaggcggtttctgctattccggagagcattcaaacagttggacgtagaagcagctctgaccaaagcccttggggaagtggacattcttctgacctggatgcagaaattctacaagctctgaatgtctagaccaggacctccctccccctggcactggtttgttccctgtgtcatttcaaacagtctcccttcctatgctgttcactggacacttcacgcccttggccatgggtcccattcttggcccaggattattgtcaaagaagtcattctttaagcagcgccagtgacagtcagggaaggtgcctctggatgctgtgaagagtctacagagaagattcttgtatttattacaactctatttaattaatgtcagtatttcaactgaagttctatttatttgtgagactgtaagttacatgaaggcagcagaatattgtgccccatgcttctttacccctcacaatccttgccacagtgtggggcagtggatgggtgcttagtaagtacttaataaactgtggtgctttttttggcctgtctttggattgttaaaaaacagagagggatgcttggatgtaaaactgaacttcagagcatgaaaatcacactgtctgctgatatctgcagggacagagcattggggtgggggtaaggtgcatctgtttgaaaagtaaacgataaaatgtggattaaagtgcccagcacaaagcagatcctcaataaacatttcatttcccacccacactcgccagctcaccccatcatccctttcccttggtgccctccttttttttttatcctagtcattcttccctaatcttccacttgagtgtcaagctgaccttgctgatggtgacattgcacctggatgtactatccaatctgtgatgacattccctgctaataaaagacaacataactca

SEQ ID NO: 4 - protein, 206 aa, Homo sapiens
MNFQQRLQSLWTLARPFCPPLLATASQMQMVVLPCLGFTLLLWSQVSGAQGQEFHFGPCQVKGVVPQKLWEAFWAVKDTMQAQDNITSARLLQQEVLQNVSDAESCYLVHTLLEFYLKTVFKNYHNRTVEVRTLKSFSTLANNFVLIVSQLQPSQENEMFSIRDSAHRRFLLFRRAFKQLDVEAALTKALGEVDILLTWMQKFYKL

SEQ ID NO: 5 - DNA, 3160 bp, Homo sapiens
  CDS (1035)..(2246)
cctcccctcgcccggcgcggtcccgtccgcctctcgctcgcctcccgcctcccctcggtcttccgaggcgcccgggctcccggcgcggcggcggagggggcgggcaggccggcgggcggtgatgtggcaggactctttatgcgctgcggcaggatacgcgctcggcgctgggacgcgactgcgctcagttctctcctctcggaagctgcagccatgatggaagtttgagagttgagccgctgtgaggcgaggccgggctcaggcgagggagatgagagacggcggcggccgcggcccggagcccctctcagcgcctgtgagcagccgcgggggcagcgccctcggggagccggccggcctgcggcggcggcagcggcggcgtttctcgcctcctcttcgtcttttctaaccgtgcagcctcttcctcggcttctcctgaaagggaaggtggaagccgtgggctcgggcgggagccggctgaggcgcggcggcggcggcggcggcacctcccgctcctggagcgggggggagaagcggcggcggcggcggccgcggcggctgcagctccagggagggggtctgagtcgcctgtcaccatttccagggctgggaacgccggagagttggtctctccccttctactgcctccaacacggcggcggcggcggcggcacatccagggacccgggccggttttaaacctcccgtccgccgccgccgcaccccccgtggcccgggctccggaggccgccggcggaggcagccgttcggaggattattcgtcttctccccattccgctgccgccgctgccaggcctctggctgctgaggagaagcaggcccagtcgctgcaaccatccagcagccgccgcagcagccattacccggctgcggtccagagccaagcggcggcagagcgaggggcatcagctaccgccaagtccagagccatttccatcctgcagaagaagccccgccaccagcagcttctgccatctctctcctcctttttcttcagccacaggctcccagacatgacagccatcatcaaagagatcgttagcagaaacaaaaggagatatcaagaggatggattcgacttagacttgacctatatttatccaaacattattgctatgggatttcctgcagaaagacttgaaggcgtatacaggaacaatattgatgatgtagtaaggtttttggattcaaagcataaaaaccattacaagatatacaatctttgtgctgaaagacattatgacaccgccaaatttaattgcagagttgcacaatatccttttgaagaccataacccaccacagctagaacttatcaaacccttttgtgaagatcttgaccaatggctaagtgaagatgacaatcatgttgcagcaattcactgtaaagctggaaagggacgaactggtgtaatgatatgtgcatatttattacatcggggcaaatttttaaaggcacaagaggccctagatttctatggggaagtaaggaccagagacaaaaagggagtaactattcccagtcagaggcgctatgtgtattattatagctacctgttaaagaatcatctggattatagaccagtggcactgttgtttcacaagatgatgtttgaaactattccaatgttcagtggcggaacttgcaatcctcagtttgtggtctgccagctaaaggtgaagatatattcctccaattcaggacccacacgacgggaagacaagttcatgtactttgagttccctcagccgttacctgtgtgtggtgatatcaaagtagagttcttccacaaacagaacaagatgctaaaaaaggacaaaatgtttcacttttgggtaaatacattcttcataccaggaccagaggaaacctcagaaaaagtagaaaatggaagtctatgtgatcaagaaatcgatagcatttgcagtatagagcgtgcagataatgacaaggaatatctagtacttactttaacaaaaaatgatcttgacaaagcaaataaagacaaagccaaccgatacttttctccaaattttaaggtgaagctgtacttcacaaaaacagtagaggagccgtcaaatccagaggctagcagttcaacttctgtaacaccagatgttagtgacaatgaacctgatcattatagatattctgacaccactgactctgatccagagaatgaaccttttgatgaagatcagcatacacaaattacaaaagtctgaatttttttttatcaagagggataaaacaccatgaaaataaacttgaataaactgaaaatggacctttttttttttaatggcaataggacattgtgtcagattaccagttataggaacaattctcttttcctgaccaatcttgttttaccctatacatccacagggttttgacacttgttgtccagttgaaaaaaggttgtgtagctgtgtcatgtatatacctttttgtgtcaaaaggacatttaaaattcaattaggattaataaagatggcactttcccgttttattccagttttataaaaagtggagacagactgatgtgtatacgtaggaattttttccttttgtgttctgtcaccaactgaagtggctaaagagctttgtgatatactggttcacatcctacccctttgcacttgtggcaacagataagtttgcagttggctaagagaggtttccgaaaggttttgctaccattctaatgcatgtattcgggttagggcaatggaggggaatgctcagaaaggaaataattttatgctggactctggaccatataccatctccagctatttacacacacctttctttagcatgctacagttattaatctggacattcgaggaattggccgctgtcactgcttgttgtttgcgcatttttttttaaagcatattggtgctagaaaaggcagctaaaggaagtgaatctgtattggggtacaggaatgaaccttctgcaacatcttaagatccacaaatgaagggatataaaaataatgtcataggtaagaaacacagcaacaatgacttaaccatataaatgtggaggctatcaacaaagaatgggcttgaaacattataaaaattgacaatgatttattaaatatgttttctcaattgtaaaaaaaaaa

SEQ ID NO: 6 - protein, 403 aa, Homo sapiens [sequence omitted]

SEQ ID NO: 7 - protein, 61 aa, Artificial Sequence
  MOD_RES (13): Xaa - anything
MARYRHSRSRSRXRYRRRRRRRSRYRSRRRRYRGSRRSRSRRRGRRRGYSRRRYSRRRRRY

SEQ ID NO: 8 - protein, 61 aa, Artificial Sequence
  Description of Artificial Sequence  Synthetic Peptide
MARYRHSRSRSRSRYRRRRRRRSRYRSRRRRYRGSRRRRRSRRRRRRGYSRRRYSRRRRRY

SEQ ID NO: 9 - protein, 62 aa, Artificial Sequence
  Description of Artificial Sequence  Synthetic Peptide
MARYRHSRSRSRSRYRRRRRRRSRYRSRRRRYRGSRRRRSRRRGRRRGYSRRRYSRRRRRRY

SEQ ID NO: 10 - protein, 64 aa, Artificial Sequence
  Description of Artificial Sequence  Synthetic Peptide
MARYRHSRSRSRSRYRRRRRRRSRYRGRRRRYRRSRRRRRSRRGRRGYYRRRRYSRRRRRRYYY

SEQ ID NO: 11 - protein, 65 aa, Artificial Sequence
  Description of Artificial Sequence  Synthetic Peptide
MARYRHSRSRSRSGYRRQRRRRSRYRSRRRRYRRRQRRSRRGRRRGYSRRRYSRRRYSRRRRRRY

SEQ ID NO: 12 - protein, 61 aa, Artificial Sequence
  Description of Artificial Sequence  Synthetic Peptide
MARYRHSRSRSRSRYRRRRRRRSRYRSRRRRYRGRRRRRSRRGRRRGYSRRRYSRRRRRRY

SEQ ID NO: 13 - protein, 61 aa, Artificial Sequence
  Description of Artificial Sequence  Synthetic Peptide
MARYRHSRSRSRSXYRRRXRRRSRYRSRRRRYRGRRRRRSRRGRRRGYSRRRYSRRRRRRY

SEQ ID NO: 14 - protein, 61 aa, Artificial Sequence; identical to SEQ ID NO: 12
  Description of Artificial Sequence  Synthetic Peptide

SEQ ID NO: 15 - protein, 62 aa, Artificial Sequence
  Description of Artificial Sequence  Synthetic Peptide
MARYRHSRSRSRSRYRRRRRRRSRYRSRRRRYRGRRRRRSRRGRRRRGYSRRRYSRRRRRRY

SEQ ID NO: 16 - protein, 62 aa, Artificial Sequence
  Description of Artificial Sequence  Synthetic Peptide
MARYRHSRSRSRSRYRRRRRRRSRYRSRRRXYRGRRRRRSRRGRRRRGYSRRRYSRRRRRRY

SEQ ID NO: 17 - protein, 60 aa, Artificial Sequence
  Description of Artificial Sequence  Synthetic Peptide
MARYRHSRSRSRSRYRRRRRRRSRYRSRRRRXRRRRRSRRGRRRRGYSRRRYSRRRRRRY

SEQ ID NO: 18 - protein, 61 aa, Artificial Sequence; identical to SEQ ID NO: 12
  Description of Artificial Sequence  Synthetic Peptide

SEQ ID NO: 19 - protein, 62 aa, Artificial Sequence
  Description of Artificial Sequence  Synthetic Peptide
XARYRHSRSRXRSRYRRRRRXRSRYRSXRRRYRGRRRRRSRRGRRRRGYSRXRYSRRRRRRY

SEQ ID NO: 20 - protein, 62 aa, Artificial Sequence
  Description of Artificial Sequence  Synthetic Peptide
MARYRHSXSRSRSRYRRRRRRRSRYRSRRRRYRGRRRRRSRRGRRRRGYSRRRYSRRRRRRY

SEQ ID NO: 21 - protein, 62 aa, Artificial Sequence
  Description of Artificial Sequence  Synthetic Peptide
MARYRHSRSRSRSRYRRRRRRRSRYRSRRRRYRGRRRRRSRRGRRRRGYSCRRYSRRRRRRY

SEQ ID NO: 22 - protein, 61 aa, Artificial Sequence
  Description of Artificial Sequence  Synthetic Peptide
MARYRHSRSRSRSRYRRRRRRRSRYRSRRRRYRGRRRXRSRRGRRRGYSRRRYSRRRRRRY

SEQ ID NO: 23 - protein, 63 aa, Artificial Sequence
  Description of Artificial Sequence  Synthetic Peptide
MARYRRHSRSRSRSRYRRRRRRRSRHHNRRRTYRRSRRHSRRRRGRRRGYSRRRYSRRGRRRY

SEQ ID NO: 24 - protein, 63 aa, Artificial Sequence; identical to SEQ ID NO: 23
  Description of Artificial Sequence  Synthetic Peptide

SEQ ID NO: 25 - protein, 63 aa, Artificial Sequence; identical to SEQ ID NO: 23
  Description of Artificial Sequence  Synthetic Peptide

SEQ ID NO: 26 - protein, 63 aa, Artificial Sequence; identical to SEQ ID NO: 23
  Description of Artificial Sequence  Synthetic Peptide

SEQ ID NO: 27 - DNA, 219 bp, Oncorhynchus mykiss
  CDS (15)..(110)
atctatcaatcactatgcccagaagacgcagagccagccgccgtatccgcaggcgccgtcgccccagggtgtcccggcgtcgcaggggaggccgccgcaggaggcgttagacaggccgggtaacctacctgaactaaccgccccctaccggccggttctccctccagactcgaccactggtagtgcagagatgttaaaagtctgcttaaataaaagatg

SEQ ID NO: 28 - protein, 31 aa, Oncorhynchus mykiss
MPRRRRASRRIRRRRRPRVSRRRRGGRRRRR

SEQ ID NO: 29 - DNA, 2567 bp, Oncorhynchus keta
  CDS (2069)..(2170)
gaattctggcattgctaggagagagtcagagtagggcctcttgaacctctgcacaggtgtccctctggtcccgcccactaggctgtggaaactaacctgtcaacattctctgctgccaatgactggaacgaactgcaaaaatcactgaacttggatacccatatctccctttaagcaccagctgtcagagcagctcacaaatcactgcacctgtaaatagcccatctgctaaacagcccatccaactacctcattcccatactgcatccatttattcatcttgctcctttgcaccccagtatctctacatgcacattaatcttctgcacatctaccattccagtgttctatttgctatattgtaattacttagccactatggcctatttgttgctttacctatttgttgcttacctcccttattttacctcatttgccactcactgtatatagattttttctactgtattatttattgactgtatgtttgtttattccatgtgtaactctgtgttgttgttggtgtcgaactgcggtgctttatcttggccagtcgcagttgtaaatgagaacttgttctcaccttgcctacctgttaaataaaggtaaaataaaaaagtgtcaatcacttgtcatggtatccagtggagtgggctcctgaagatctaccattttagagagcattctctctatattgagatcaaataaaatatgtagaggatgaaatgtttgactgagtttattatttgggaaatgacttttacattataccccattctgataacaatctactgtagagcagcatttgatgatcataatatgacttgctttatgcacaacttgttgtgtgccattaaatcacaatgcagtttcagtgacatcacaacattctgattctgagaggctggttgcagtagttacacagacattctgaacagtttagctgaaagaagctgattcaattgactccgagaaatcacatgataatagcatgtaatgagagcgcctactcggtggtttagagattggttgtaataaacatatttacggtggtttcagactttctaatggatgacatggctgacatgtcaagggtaatagtagtagaggtcattaataattactgcagtgggctgaatcagggtcacacagtgtttctaggtagtcttaaaactactttcagacaaaagtatacacctcacacacatggttatgggtgtgaggtgtatacagaagacacctacctacctgtaccatgtcagagatagagttgatagagttgtattacatgttgagtttgcatcccaatatgacactttatatacatcacagaagactgaaatataacaaaattgtttgacatagaaacaccggattttcggcagcttttaaaaaaataatgtgtattaattatgaaatgatgaatcatattaatgtcattccacccatgaggctactaggttatttgactgcaggaaattgatgattaaatagactttccttaaatcctctgttctgttttggcataatcaaccgaagatgtgttttactgtagtatgatagcctatctgtattataatatgctagcattctatgctgcagtaggatctcctacaacattccaaatcaccattaaataaagacctgttgattatttcttccatggttcattgtgttggccaaataaacagattgttatgggtgtaagatggcagcacagtgatgtcatctgagttggtaaatgttcattactgcaactcgtgtgttttaccggttttacccggatgtaattatgatgtactgaacaagactggttactcgcatcaatggccctgtctcgtcatttaacattcaaacacagatcgatttaaaatgacaaaataaaaatatcattattgcaccatcctgccactgctactatgacgtcataattcagatgtcttctcaatttaaactgtctttaatacttattgcatcattatttatcccataatgacatcactccagctcccctccagccctataaaagggacaaccgcctgtctaaaatgtctatccatcaatcacaatgcccagaagacgcagatcctccagccgacctgtccgcaggcgccgccgccctagggtgtcccgacgtcgtcgcaggagaggaggccgcaggaggcgttagataggacgggtagaaccacctgacctatccgccccctccgggttctccctcccgacccttggtagtgtagaggtgttaaagtctgcttaaataaaagatgggttttaactaaaactgttacgactttatattagtagataggtttttttaggctgtaagagtttttggcgatggagttaataatatatttgagataatacaataatagcctactatgttagtaatatatttaattaaaacgttttaataattgtactgtccctaataaataaatacattaaaacaacatatttattgaaaacagtgacacattcaatcgtcaagtcagataatgctttgtaccattatggtttagtttgcgctcattttcagcatacatctagtcatttctggatcc

SEQ ID NO: 30 - protein, 33 aa, Oncorhynchus keta
MPRRRRSSSRPVRRRRRPRVSRRRRRRGGRRRR

SEQ ID NO: 31 - DNA, 1095 bp, Homo sapiens
  CDS (363)..(806)
tccccgctctgctctgtccggtcacaggactttttgccctctgttcccgggtccctcaggcggccacccagtgggcacactcccaggcggcgctccggccccgcgctccctccctctgcctttcattcccagctgtcaacatcctggaagctttgaagctcaggaaagaagagaaatccactgagaacagtctgtaaaggtccgtagtgctatctacatccagacggtggaagggagagaaagagaaagaaggtatcctaggaatacctgcctgcttagaccctctataaaagctctgtgcatcctgccactgaggactccgaagaggtagcagtcttctgaaagacttcaactgtgaggacatgtcgttcagatttggccaacatctcatcaagccctctgtagtgtttctcaaaacagaactgtccttcgctcttgtgaataggaaacctgtggtaccaggacatgtccttgtgtgcccgctgcggccagtggagcgcttccatgacctgcgtcctgatgaagtggccgatttgtttcagacgacccagagagtcgggacagtggtggaaaaacatttccatgggacctctctcaccttttccatgcaggatggccccgaagccggacagactgtgaagcacgttcacgtccatgttcttcccaggaaggctggagactttcacaggaatgacagcatctatgaggagctccagaaacatgacaaggaggactttcctgcctcttggagatcagaggaggaaatggcagcagaagccgcagctctgcgggtctactttcagtgacacagatgtttttcagatcctgaattccagcaaaagagctattgccaaccagtttgaagaccgcccccccgcctctccccaagaggaactgaatcagcatgaaaatgcagtttcttcatctcaccatcctgtattcttcaaccagtgatcccccacctcggtcactccaactcccttaaaatacctagacctaaacggctcagacaggcagatttgaggtttccccctgtctccttattcggcagccttatgattaaacttccttctctgctgcaaaaaaaaaaaaaaa

SEQ ID NO: 32 - protein, 147 aa, Homo sapiens
MSFRFGQHLIKPSVVFLKTELSFALVNRKPVVPGHVLVCPLRPVERFHDLRPDEVADLFQTTQRVGTVVEKHFHGTSLTFSMQDGPEAGQTVKHVHVHVLPRKAGDFHRNDSIYEELQKHDKEDFPASWRSEEEMAAEAAALRVYFQ

(19) United States
(12) Patent Application Publication
(10) Pub. No.: US 2004/0028654 A1
(43) Pub. Date: Feb. 12, 2004

(54) Protamine-adenoviral vector complexes and methods of use

(75) Inventors: Lin Ji, Sugar Land, TX (US); Jack Roth, Houston, TX (US)

Correspondence Address: Gina N. Shishima, Fulbright & Jaworski L.L.P., Suite 2400, 600 Congress Avenue, Austin, TX, 78701, US

(73) Assignee: Board of Regents, The University of Texas System

(21) Appl. No.: 10/391,068

(22) Filed: Mar. 24, 2003

Related U.S. Application Data
(60) Provisional application No. 60/366,846, filed on Mar. 22, 2002.

(51) Int. Cl. A61K 48/00

(52) U.S. Cl. 424/93.2; 514/2

(57) ABSTRACT

Embodiments of the invention include methods and compositions including viral composition that have high transduction efficiencies in vivo, in vitro and ex vivo. The viral composition include a viral vector and a protamine molecule, wherein the viral vector includes a polynucleotide encoding a tumor suppressor gene. The methods of the invention include administering the viral composition to a patient or subject for treatment of disease, in particular cancer, that is characterized by a reduced vector-induced production of neutralizing antibodies and a decreased vector-induced toxicity as compared to delivery of viral vectors alone.

This application claims priority to U.S. Provisional Patent application serial No. 60/366,846 filed on Mar. 22, 2002, which is incorporated herein by reference.

The United States government may own rights in the present invention pursuant to grant numbers 2P50-CA70970-04 and CA78778-01A1 from the National Institutes of Health.

### BACKGROUND OF THE INVENTION

I. Field of the Invention

The present invention relates generally to the fields of oncology, molecular biology, and virology. More particularly, it concerns methods and compositions for the prophylactic and therapeutic treatment of hyperproliferative disorders using a viral composition for transduction of a transgene to a cell, in particular to a cancer cell.

II. Description of Related Art

Advances in understanding and manipulating genes have set the stage for scientists to alter or augment a patients' genetic material to fight or prevent disease, i.e., Gene Therapy. Various clinical trials using gene therapies have been initiated and have included the treatment of various cancers, AIDS, cystic fibrosis, adenosine deaminase deficiency, cardiovascular disease, Gaucher's disease, rheumatoid arthritis, and others.

The primary modality for the treatment of cancer using gene therapy is the induction of apoptosis. This can be accomplished by either sensitizing a cancer cell to an agent or inducing apoptosis directly by stimulating intracellular pathways. Other cancer therapies take advantage of the need for a tumor to induce angiogenesis to supply the growing tumor with necessary nutrients, e.g., endostatin and angiostatin therapies (WO 00/05356 and WO 00/26368).

One of the various goals of gene therapy is to supply cells with a nucleic acid encoding a functional protein to restore or provide an activity of a missing or altered protein, thereby altering the genetic makeup of some of the patient's cells. One mode of delivery for genetic material involves the use of viruses that are genetically disabled and unable to reproduce themselves. Other delivery systems include non-viral vectors and direct delivery of expression vectors (e.g., naked DNA).

Most gene therapy clinical trials rely on mouse retroviruses to deliver the desired gene, but other vectors include adenoviruses, adeno-associated viruses, pox viruses, polyoma virus and the herpes virus. Liposomes have also been used as a vector in gene therapy. Currently, adenovirus is the preferred vehicle for delivery of gene therapy agents because, relative to the other viral vectors, an adenovirus provides higher transduction efficiencies, infection of non-dividing cells, easy manipulation of its genome, and a lower probability of non-homologous recombination with the host genome.

Studies have been reported that attempt to improve the therapeutic potential of adenoviral based gene delivery systems. In particular, Lanuti et al have published studies that investigate the effect of protamine augmented adenovirus-mediated cancer gene therapy. Lanuti et al. report studies that show an increased efficiency of adenovirus mediated gene transfer and potentiation of cytotoxic effects in vitro. The authors also report that the administration of protamine with adenovirus increases the efficiency of adenovirus mediated gene transfer to a tumor target in vivo. However, the authors failed to observe any increase in treatment efficacy of protamine augmented adenovirus therapy in vivo.

Clinical investigations have shown that there are few adverse effects associated with the viral vectors (Anderson et al., 1992), but it would be of great benefit to improve the clinical efficacy of gene therapy, and in particular, of viral vectors carrying anticancer or anti-proliferative genes. Thus, there is a need for improved methods and compositions for viral mediated gene delivery.

### SUMMARY OF THE INVENTION

The invention includes methods and compositions that can be used in the prophylactic and therapeutic treatment of cancer and other hyperproliferative diseases, for example lung cancer. Methods and compositions of the invention involve a viral composition that can be administered systemically. Embodiments of the invention include viral compositions having improved transduction efficiency in vitro, ex vivo, and in vivo. In certain embodiments, the methods provide for an increased transduction efficiency and therapeutic efficacy in cancer cells and tumors, in particular cancer and tumor cells associated with the lung. Certain embodiments of the invention include viral compositions comprising a (a) a protamine molecule and (b) a therapeutic viral vector.

Protamine is a natural, arginine-rich peptide with an overall positive charge. In certain embodiments, the protamine molecule is typically complexed with the viral vector through electrostatic attraction to the negatively charged surface of the viral vector. The term “protamine molecule,” as used herein, refers to low molecular weight cationic, arginine-rich polypeptide. The protamine molecule typically comprises about 20, 25, 30, 35, 40, 45, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 115, 120, 125, 130, 135, 140, 145, 150, 175, to about 200 or more amino acids and is characterized by containing at least 20%, 30%, 40%, 50%, 60%, 70% arginine. It is contemplated that at least 1, 2, 3, 4, 5, 6, 7, 8, 9, 10 or more protamine molecules may be complexed with each viral vector.

A viral vector and protamine molecule complex can be used for increasing transduction efficiencies, increasing therapeutic efficacy and alleviating side effects of viral vector therapy, such as neutralizing antibody production and hepatic toxicity. In certain embodiments of the invention, viral vector and protamine complexes include a ratio of viral vector to protamine of about 10¹⁰, about 10¹¹, about 10¹², about 10¹³, about 10¹⁴, or about 10¹⁵ viral particles or plaque forming units (pfu) to about 50, 55, 60, 65, 70, 75, 80, 85, 90, 95, 100, 105, 110, 115, 120, 125, 130, 135, 140, 145, 150, 200, 250, or 300 μg protamine.

In certain embodiments, a targeting moiety or ligand may be operably coupled to a protamine molecule. The term “targeting moiety” or “targeting ligand,” as used herein refers to a molecule or moiety having the characteristic, property or activity of directing transportation or localization of the viral composition to a specific site, location or cell type. A targeting moiety can be, for example, a peptide, a polypeptide, an oligonucleotide, a polynucleotide, a detectable label, or a drug. Polypeptides may include, but are not limited to enzymes, antibodies, antibody fragments (e.g., single chain antibodies), protein-protein interaction domains, ligands for cell surface receptors, cytokines, growth factors, hormones, toxins, and/or inducers of apoptosis. In certain embodiments, the targeting moiety is a peptide or a polypeptide. In specific embodiments, the targeting moiety is a ligand, such as a peptide ligand that interacts with cell surface receptors, such as EGFR, VEGFR, and CAR. The targeting moiety may also be a tissue and/or cell-specific ligand, such as uPA, heparin, AKAP, and hemagglutin, and so on.

The targeting ligand may be operably coupled to a protamine molecule either directly, e.g., a fusion protein, or indirectly by means of a linking moiety. Generally, the term “linking moiety” refers to a molecule or moiety having a chemical or physical property of linking or being able to link two or more moieties, thereby conjugating or operably coupling two or more moieties, for example, protamine and a targeting peptide. In some embodiments, the linking moiety may react and bind the guanidino group of the arginine side-chain. In certain embodiments, the linking moiety is salicylhydroxamic acid (SHA). Other suitable linking moieties may also be considered within the scope of the present invention, including but not limited to SHA, FDNB, DNP, phenyglyoxal, a diene, iodoacetate, diethylpyrocarbonate, succinic anhydride, ethylmaleimide, and succinimide. The linking moiety may directly bind, bond, attach, and/or coordinate a targeting peptide to a protamine molecule. A protamine-peptide conjugate may be complexed with a viral vector in the same manner as discussed below.

In some embodiments, the linking moiety may couple the protamine and/or the targeting moiety to the viral vector.

In certain embodiments, a fusion protein includes protamine fused to a targeting moiety such as a peptide ligand, an antibody or the like.

Embodiments of the invention include a viral vector comprising an expression vector and/or an expression cassette. In certain embodiments, the viral vector is an adenoviral vector, a retroviral vector, a vaccinia viral vector, an adeno-associated viral vector, a polyoma viral vector, or a herpes viral vector. The viral vector may be a replication-competent, conditionally-replicating, replication-restricted, or replicationdeficient viral vector.

“Replication-competent” as applied to a vector means that the vector is capable of replicating in normal and/or neoplastic cells. As applied to a recombinant virus, “replication-competent” means that the virus exhibits the following phenotypic characteristics in normal and/or neoplastic cells: cell infection; replication of the viral genome; and production and release of new virus particles; although one or more of these characteristics need not occur at the same rate as they occur in the same cell type infected by a wild-type virus, and may occur at a faster or slower rate. Where the recombinant virus is derived from a virus such as adenovirus that lyses the cell as part of its life cycle, it is preferred that at least 5 to 25% of the cells in a cell culture monolayer are dead 5 days after infection. Preferably, a replication-competent virus infects and lyses at least 25 to 50%, more preferably at least 75%, and most preferably at least 90% of the cells of the monolayer by 5 days post infection (p.i.).

“Replication-defective” as applied to a recombinant virus means the virus is incapable of, or is greatly compromised in, replicating its genome in any cell type in the absence of a complementing replication-competent virus. Exceptions to this are cell lines such as 293 cells that have been engineered to express adenovirus E1A and E1B proteins.

The term “conditionally-replicating” refers to a viral vector that will replicate under certain conditions, but not others, i.e., a conditionally replicating vector can only replicate in particular cells and/or under particular conditions. In particular, “Replicationrestricted” as applied to a vector of the invention means the vector replicates better in a dividing cell, i.e., either a neoplastic cell or a non-neoplastic, dividing cell, than in a cell of the same type that is not neoplastic and/or not dividing, which is also referenced herein as a normal, non-dividing cell. Preferably, a replication-restricted virus kills at least 10% more neoplastic cells than normal, non-dividing cells in cell culture monolayers of the same size, as measured by the number of cells showing cytopathic effects (CPE) at 5 days p.i. More preferably, between 25% and 50%, and even more preferably, between 50% and 75% more neoplastic than normal cells are killed by a replication-restricted virus. Most preferably, a replication-restricted adenovirus kills between 75% and 100% more neoplastic than normal cells in equal sized monolayers by 5 days p.i.

Certain embodiments of the invention include a vector that is replication-competent in neoplastic cells and which overexpresses an adenoviral death protein (ADP). Vectors useful in the invention include, but are not limited to plasmid-expression vectors, bacterial vectors such as Salmonella species that are able to invade and survive in a number of different cell types, vectors derived from DNA viruses such as human and non-human adenoviruses, adenovirus associated viruses (AAVs), poxviruses, herpesviruses, and vectors derived from RNA viruses such as retroviruses and alphaviruses. Preferred vectors include recombinant viruses engineered to overexpress an ADP. Recombinant adenoviruses are particularly preferred for use as the vector, especially vectors derived from Ad1, Ad2, Ad5 or Ad6.

Vectors according to the invention may or may not overexpress ADP. As applied to recombinant Ad and AAV vectors, the term “overexpresses ADP” means that more ADP molecules are made per viral genome present in a dividing cell infected by the vector than expressed by any previously known recombinant adenoviral vector or AAV in a dividing cell of the same type. In certain embodiments, an adenovirus may overexpress the adenovirus death protein (ADP). A therapeutic adenovirus may exhibit an upregulated expression of ADP relative to wild-type adenovirus.

As applied to other, non-adenoviral vectors, “overexpresses ADP” means that the virus expresses sufficient ADP to lyse a cell containing the vector.

In various embodiments, the viral vector is an adenoviral vector. The adenoviral vector comprises a polynucleotide encoding an adenoviral expression vector. The adenoviral expression vector may lack all or part of one or more adenoviral early regions, such as E1, E1a, E1b, E2, E2a, E2b, E3, and/or E4. In certain embodiments, the adenoviral construct lacks at least part of the E1 coding region. In some embodiments, E1b coding region is deleted. An adenoviral vector lacking the E1b region may further lack all or part of the E2, E3 and/or E4 early regions, or any combination thereof.

In certain embodiments, the viral composition includes a therapeutic adenovirus that is replication competent in one or more types of human neoplastic or hyperproliferative cells. The adenovirus may or may not replicate in one or more non-neoplastic cells to the same extent that it replicates in neoplastic cells.

In certain embodiments, a viral expression vector may comprise a polynucleotide sequence encoding a tumor suppressor gene. Tumor suppressor genes include, but are not limited to p53, MDA7, PTEN, or FHIT. In some embodiments, the expression vector has a polynucleotide sequence encoding p53. In other embodiments, the expression vector has a polynucleotide sequence encoding MDA7. In certain embodiments, the expression vector has a polynucleotide sequence encoding PTEN. In some embodiments, the expression vector has a polynucleotide sequence encoding FHIT.

In certain embodiments, the tumor suppressor gene is under control of a promoter that is operable in any cell that is targeted by the methods and compositions provided herein. Suitable promoters include, but are not limited to a CMV IE, dectin-1, dectin-2, human CD11c, F4/80, SM22alpha, a MHC class II promoter, SV40, polyoma or an adenovirus 2 promoter.

The viral expression vector may further comprises an enhancer region. As used herein, “enhancers” are genetic elements that increase transcription from a promoter located at various distances from the enhancer. An expression vector may also comprise a polyadenylation signal, for example, an SV40 or bovine growth hormone polyadenylation signal.

Certain embodiments of the invention include methods of treating a malignancy or other hyperproliferative disease using a viral composition of the invention. In one embodiment, the invention is directed to a method of treating a patient having a malignancy, such as a cancer and/or tumor, comprising administering to the patient an effective amount of a viral composition. The viral composition may or may not include a polynucleotide sequence encoding a tumor suppressor gene, as described herein. The viral composition may be comprised in a pharmacologically acceptable solution. Aspects of the viral composition discussed herein are incorporated into the viral compositions used in the inventive methods and are considered applicable and within the scope of the methods. In certain embodiments of the invention the cancer is or comprises a tumor.

A viral composition may contain at least or at most about 10¹⁰, 10¹¹, 10¹², 10¹³, 10¹⁴, or 10¹⁵ viral particles. In preferred embodiments, the range that is administered is between about 10¹⁰ to about 10¹¹, or to about 10¹² viral particles. An “effective amount” refers to the amount needed to achieve a desired goal, such as inhibiting the growth of a cancer cell, reducing the mass of a tumor and/or treating a cancer. Inhibiting the growth of a cancer cell includes inducing the cell to enter apoptosis, reducing cell growth rate, inhibiting or preventing metastasis, killing the cell and/or inhibiting cell division.

Embodiments of the invention include methods comprising the systemic administration of a viral composition of the invention. Systemic administration includes, for example, intravascular, intraarterial, and intravenous injection; continuous infusion or inhalation. Other methods of administration include, but are not limited to oral, inhalation, ocular, nasal, subcutaneous, intratumoral or intramuscular routes. In certain embodiments, the administration of the viral composition is by inhalation. In such cases, the viral composition is provided as an aerosol that, for example, is generated in an aerosol application unit, an inhaler or any device that is capable of nebulizing the viral composition. The respiratory inhalation delivery mechanism is particularly useful in the case of a lung cancer patient.

In certain embodiments, administration by direct injection may be employed, particularly when treating a tumor. In cases that the patient has a malignancy that comprises a tumor, the composition of the present invention may include administering the viral composition before, after or during tumor resection. In certain embodiments, methods of the invention comprise injection of a residual tumor site. The tumor resection may be performed by bronchoscopy.

The viral compositions may be administered one or more times to a patient or subject, and includes multiple administrations. Multiple administration may be given 1, 2, 3, 4, 5, 6, 7, 8, 9, 10 or more times. Administrations may be daily, weekly, bi-weekly, monthly, bimonthly or various times in between. The composition may be administered at the same or differing doses when administered in multiple doses.

In certain embodiments, the invention provides methods of treating a cancer patient having a malignancy. The malignancy may include, but is not limited to, lung cancer, non-small cell lung carcinoma, adenocarcinoma, large-cell undifferentiated cancer, small cell lung carcinoma, squamous cell carcinoma, epithelial cell cancer, soft tissue carcinoma or Kaposi's sarcoma. The invention can also be administered to a malignancy that is a tumor cell that originates or infiltrates the breast, lung, blood, head, neck, pancreas, prostate, bone, testicle, ovary, cervix, intestines, colon, liver, bladder, brain, tongue, gum, oropharyngeal, thyroid or nerves.

In other embodiments, the inventive compositions may be administered to a patient having a pre-cancerous growth. The term “pre-cancerous growth” refers to, for example, HPV-associated growths on the cervix, or urogenitary tract including perineal, vulvar and penile growths or lesions.

The invention may also include combination treatments that comprise administering the viral composition of the present invention to a patient receiving or who will be receiving chemotherapy, radiotherapy, immune therapy including hormone therapy, other gene therapy or has undergone surgery such as a tumor resection. The compositions of the invention may be administered prior to, during or after resection of a tumor, cancerous growth, or precancerous growth. A residual tumor site may be contacted with the compositions of the invention. A successful treatment refers to treatment that removes, diminishes, decreases, inhibits or prevents cellular proliferation of the cancer cell, which includes a treatment that affects the growth by reducing its size or growth rate, or preventing its enlargement, or reducing the number of malignant or cancer cells.

Embodiments of the invention include treatment of various patients or subjects. Patients may include humans, domestic animals, such as cows, dogs, cats, pigs, horses and the like; wild animals and such.

Embodiments of the invention include methods of preparing and viral compositions prepared by the process comprising: preparing a first solution comprising a viral vector at a concentration of about 10¹⁰, about 10¹¹, about 10¹², about 10¹³, about 10¹⁴, or about 10¹⁵ viral particles per 50 μL diluent, where, in certain embodiments, the viral vector may or may not include a polynucleotide encoding a tumor suppressor gene as described herein; preparing a second solution comprising a protamine molecule in a concentration of about 100, 125, 150, 175, 200, 225, 250, 275, 300, 325, 350, 375, 400, 425, 450, 475, 500, 525, 550, 575, 600, 25, 650, 675, 700, 725, 750, 775, 800, 825, 850, 875, 900, 925, 950, 975, 1000 μg per 50 μL diluent; mixing the first solution with the second solution in a ratio of about 1:1 to form a third solution; and incubating the third solution for a time sufficient to effect coordination between the viral vector and the protamine molecule and produce the viral composition. It is contemplated that ratios of viral particles (vp) to protamine sulfate are within a range of 1×10¹⁰ to 1×10¹¹ vp/100-1000 μg protamine sulfate. In a specific embodiment where intravenous injection of the viral composition is desired, the protamine is about 300 μg or less per dose at a concentration of less than or equal to about 1.5 μg/μl. The total number of viral particles in such cases may be about 1×10¹¹, about 2×10¹¹, about 3×10¹¹, about 4×10¹¹, or about 5×10¹¹ vp.

In certain embodiments, the method further comprises the step of adding the viral composition to a pharmacologically acceptable diluent. The viral concentration may be in a range between about 1×10¹⁰ to about 2×10¹⁰, to about 3×10¹⁰, to about 4×10¹⁰, to about 5×10¹⁰, to about 6×10¹⁰, to about 7×10¹⁰, to about 8×10¹⁰, to about 9×10¹⁰, to about 1×10¹¹, to about 2×10¹¹, to about 3×10¹¹, to about 4×10¹¹, to about 5×10¹¹, to about 6×10¹¹, to about 7×10¹¹, to about 8×10¹¹, to about 9×10¹¹, or to about 1×10¹² viral particles per total volume.

Methods of the invention also include ways to express an exogenous polypeptide in a cell using viral compositions of the invention. “Exogenous polypeptide” refers to a polypeptide expressed from a nucleic acid sequence that was added to the cell, such as a viral expression vector or nucleic acid sequence contained in a viral vector administered or provided to a cell or its parent. Exogenous polypeptides that may be expressed in a cell include, but are not limited to, wild type tumor suppressors, such as p53, PTEN, FHIT, or MDA7. Other genes that may be employed according to the present invention include Rb, APC, DCC, NF-1, NF-2, WT-1, MEN-I, MEN-II, zac1, p73, VHL, MMAC1/PTEN, DBCCR-1, FCC, rsk-3, p27, p27/p16 fusions, p21/p27 fusions, anti-thrombotic genes (e.g., COX-1, TFPI), PGS, Dp, E2F, ras, myc, neu, raf, erb, fms, trk, ret, gsp, hst, abl, E1A, p300, genes involved in angiogenesis (e.g., VEGF, FGF, thrombospondin, BAI-1, GDAIF, or their receptors) and MCC.

Another embodiment of the present invention is a method of reducing vector-based toxicity in a patient having a malignancy comprising administering to the patient an effective amount of a viral composition of the invention.

Yet other embodiments provide methods of reducing production of viral vector-induced neutralizing antibody comprising administering to a patient having a malignancy an effective amount of a viral composition of the invention.

Any of the compositions described herein may be implemented in methods of the invention and vice versa. It is contemplated that any embodiment discussed with respect to an aspect of the invention may be implemented or employed in the context of other aspects of the invention.

The use of the word “a” or “an” when used in conjunction with the term “comprising” in the claims and/or the specification may mean “one,” but it is also consistent with the meaning of “one or more,” “at least one,” and “one or more than one.”

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. Illustration of a protamine-adenovirus complex.

FIG. 2. Optimization of protamine-adenovirus complex formulation using FACS.

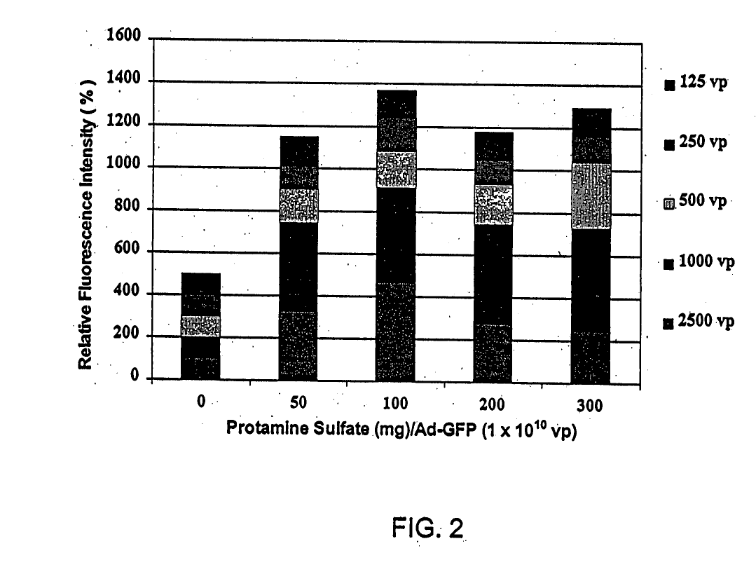

FIGS. 3A-3D. Transduction efficiency and gene expression in vitro of human NSCLC class transduced by protamine-Ad-GFP(P-Ad-GFP) and control Ad-GFP vector.

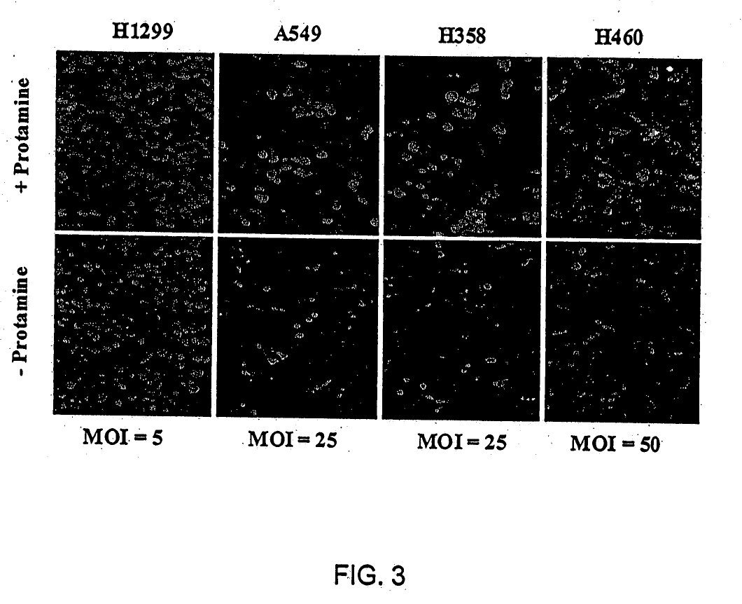

FIG. 4. Transduction efficiency of tumor cells with protamine-Ad-GFP by FACS.

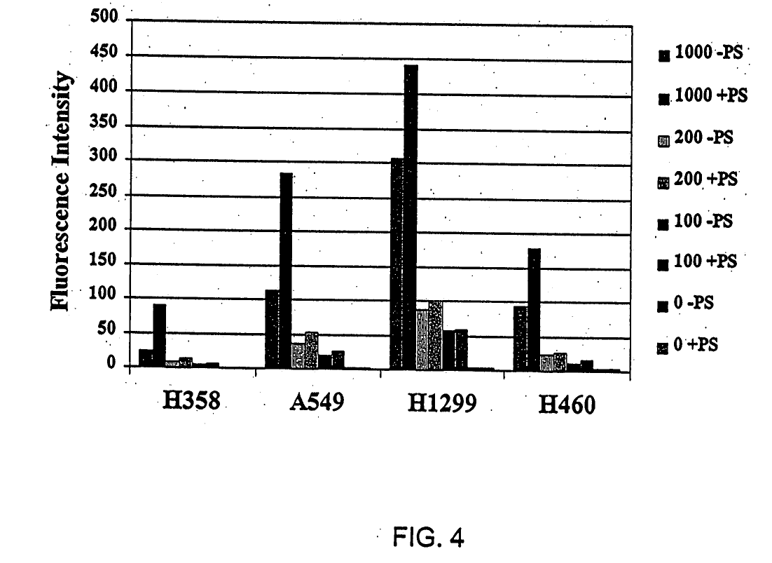

FIGS. 5A-5F. Adenoviral composition-mediated GFP expression in vivo. Intravenous administration to the lung (5C and 5D), subcutaneous tumor cells (5E and 5F) in nude mice. PBS (5A) and Ad-GFP (5B) were used as controls.

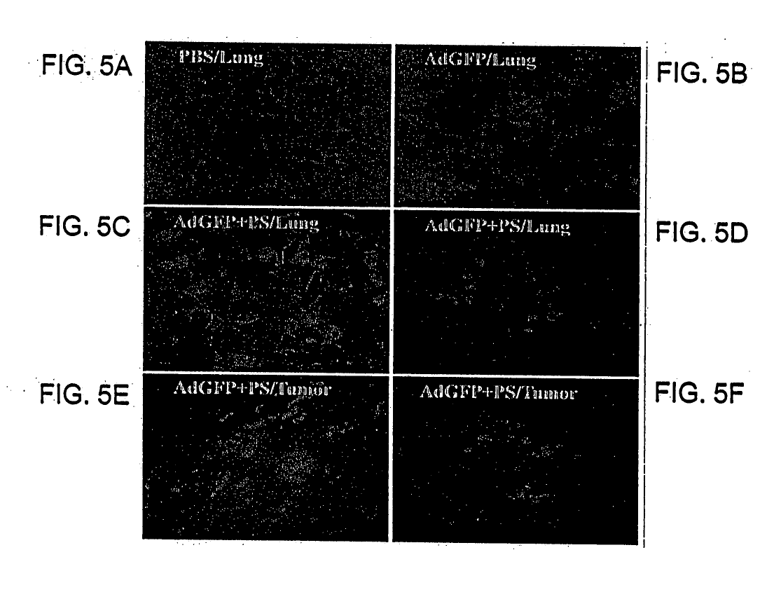

FIG. 6. Expression of GFP in vivo following administration of P-Ad-GFP and liposome-GFP complexes.

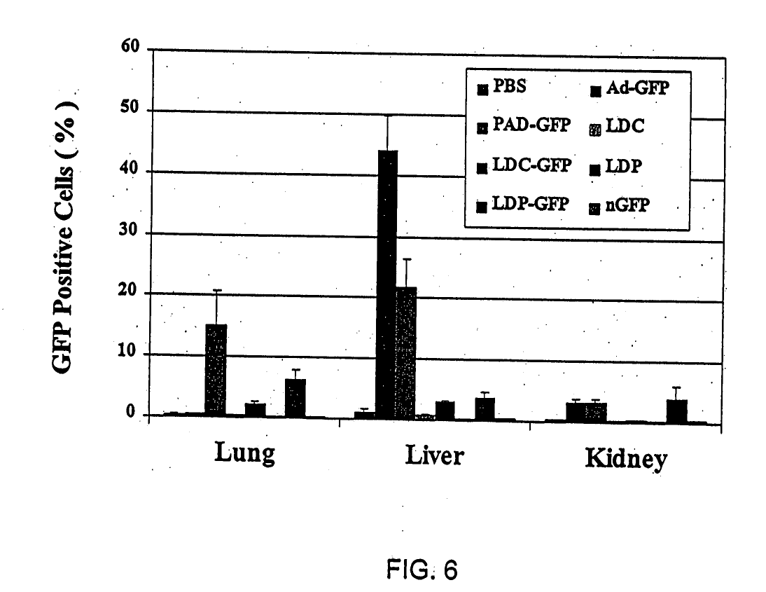

FIG. 7. Flow chart of analysis of neutralizing antibodies induced by systemic administration of adenoviral vectors.

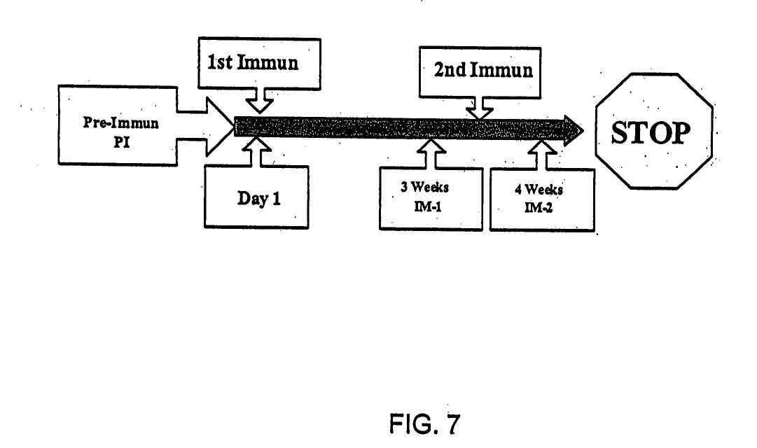

FIG. 8. Adenoviral vector-induced neutralizing antibody production in C3H mice administered PBS, protamine, Ad-GFP and P-Ad-GFP.

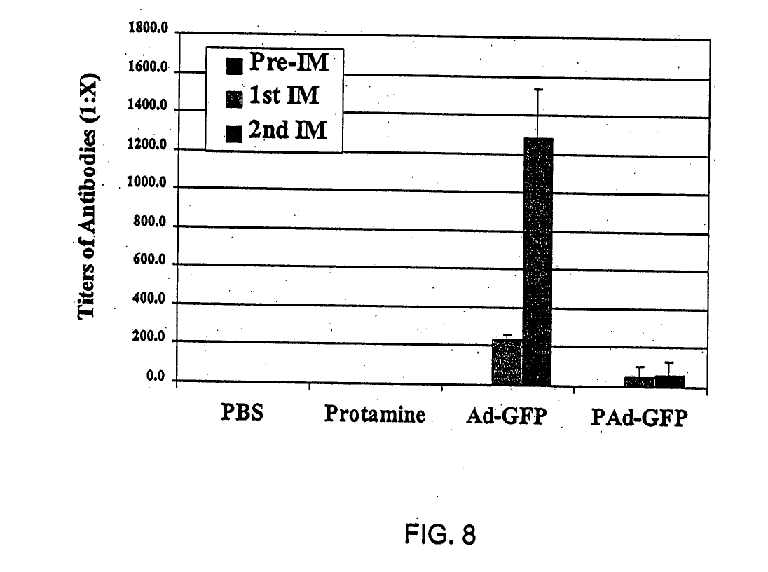

FIG. 9. Adenoviral vector-induced cytotoxicity in liver cells in animals treated with P-Ad compositions or a liposome composition.

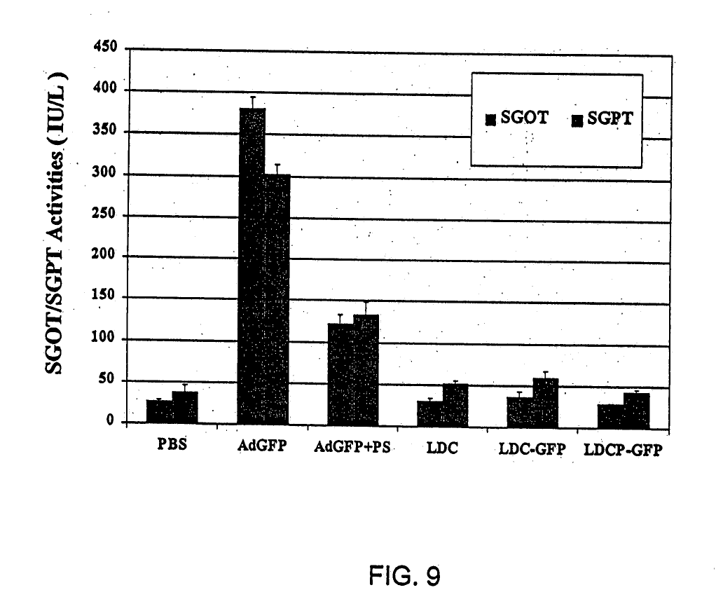

FIG. 10. Graph of tumor growth in human S2-VP 10 pancreatic tumor xenografts treated with PBD, Ad-GFP, P-Ad-p53 or P-Ad-FHIT compositions administered by intratumoral injection.

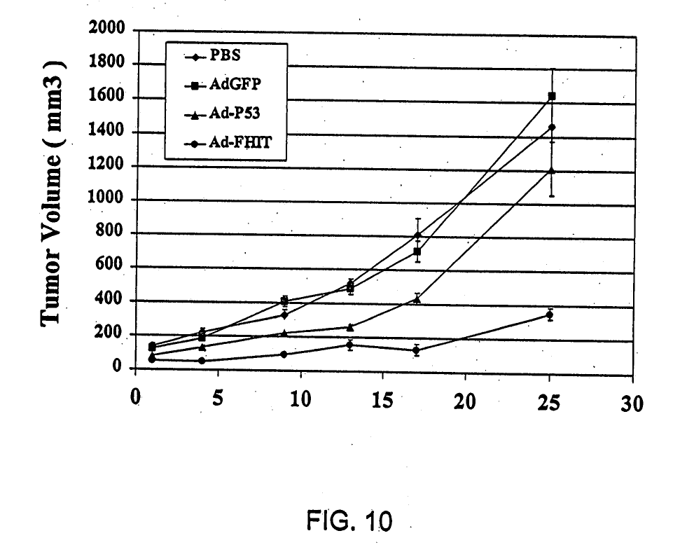

FIGS. 11A-11C. Dissections of pancreatic S2-VP10 tumors and nude mice treated with Ad-GFP (A), Ad-p53 (B), or P-Ad-FHIT (C) compositions administered by intratumoral injection.

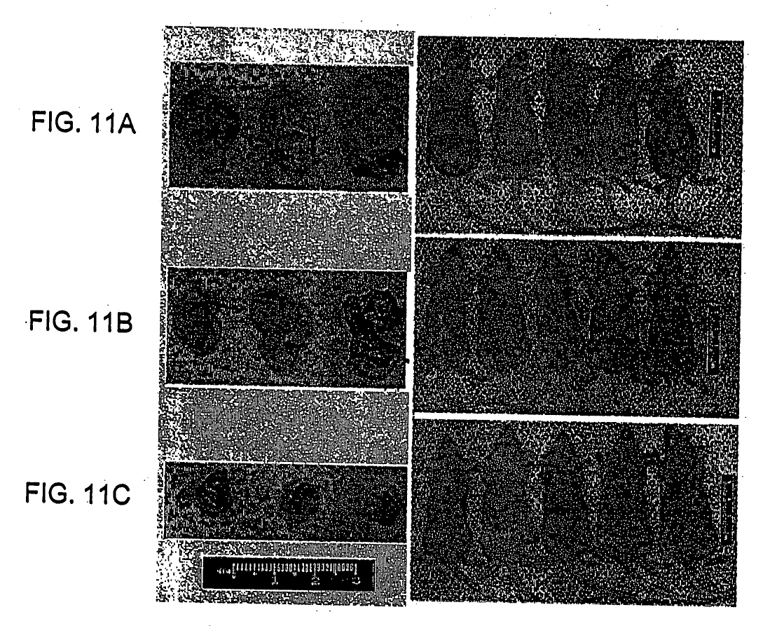

FIG. 12. Graph of relative tumor loads observed in lung metastases of S2-VP10 after systemic administration of a P-Ad-tumor suppressor gene (TSG) in nude mice.

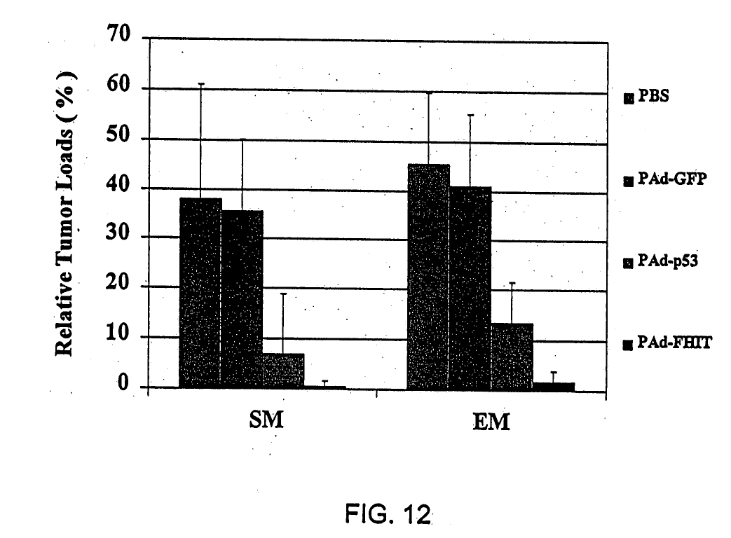

FIGS. 13A-13D. Dissections of spontaneous and experimental lung metastases of pancreatic cancers after treatment with PBS (A), P-Ad-GFP (B), P-Ad-p53 (C) or PAd-FHIT (D) compositions.

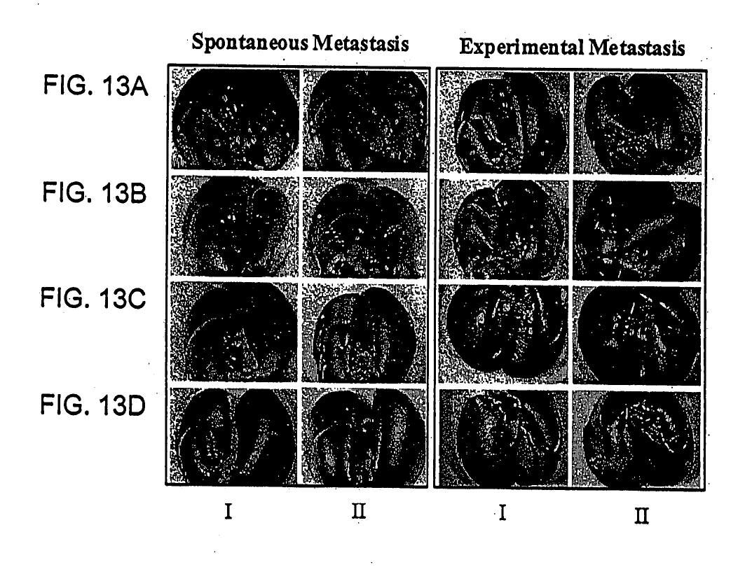

FIG. 14. Graph of therapeutic efficacy observed in systemic administration of a P-Ad-3p21.3 compositions on A549 metastases.

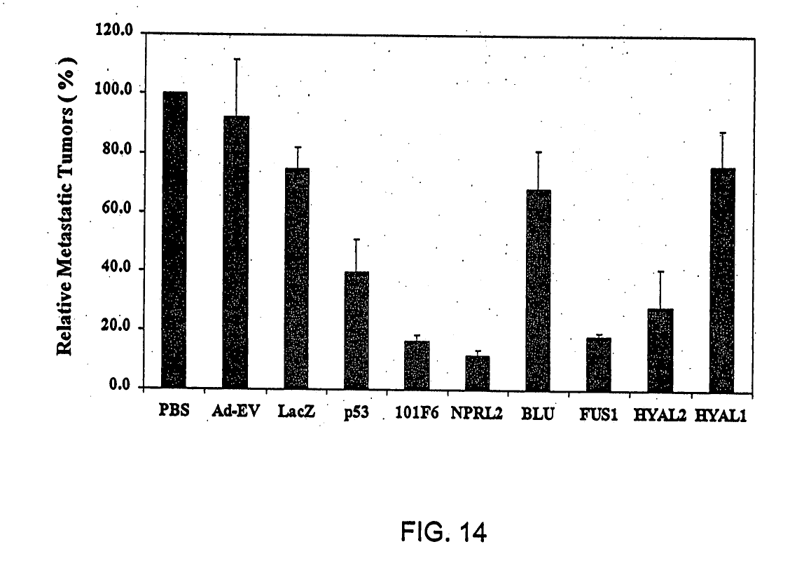

FIGS. 15A-15B. Graph of therapeutic efficacy observed in systemic administration of a P-Ad-MDA7 compositions on A549 metastases in terms of mean tumor colonies (A) and relative tumor colonies (B).

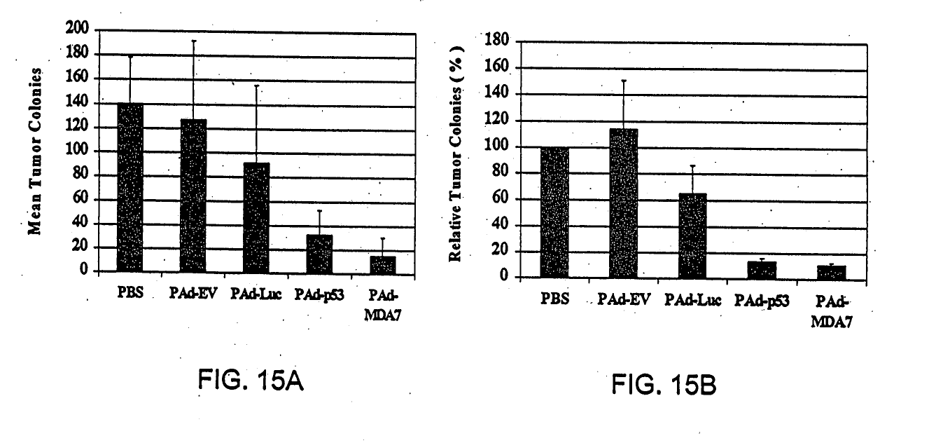

FIGS. 16A-16E. Dissections of A549 human lung metastatic tumors after systemic administration of a PBS (A), P-Ad-EV (B), P-Ad-Luc (C), P-Ad-p53 (D) and P-Ad-MDA7 compositions.

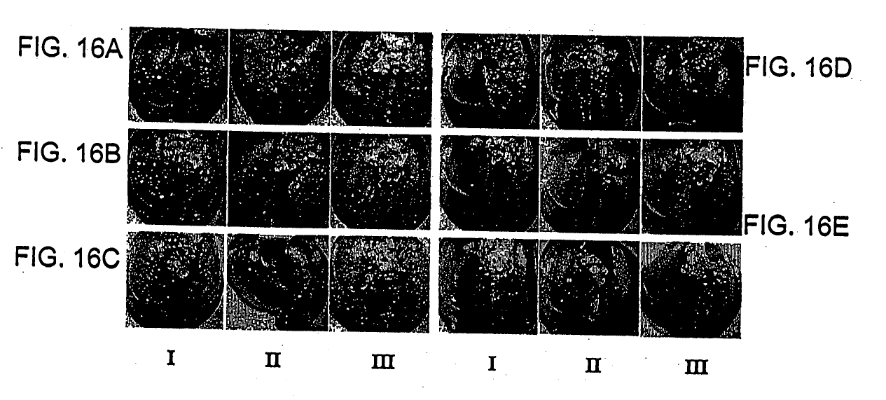

FIGS. 17A-17E. Histochemical staining of A549 human lung metastases after systemic administration of a PBS (A), P-Ad-EV (B), P-Ad-Luc (C), P-Ad-p53 (D) and P-Ad-MDA7 compositions.

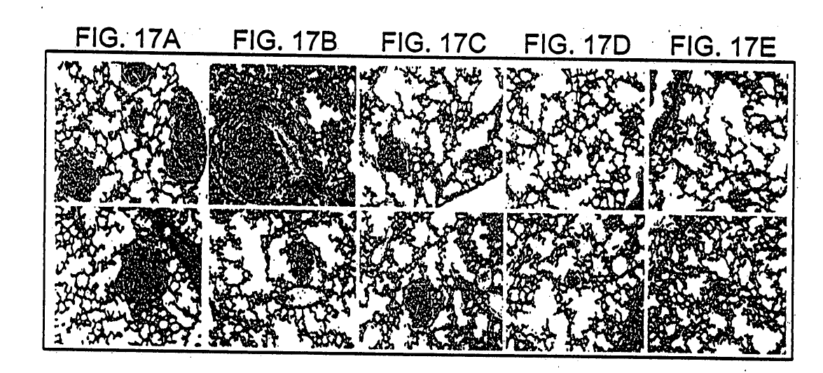

FIG. 18. Immunohistochemical staining using anti-p53 antibodies of transgene expression in mouse lung metastases tumors treated with P-Ad-EV (A), PBS (B), P-Ad-p53 (C and D) compositions.

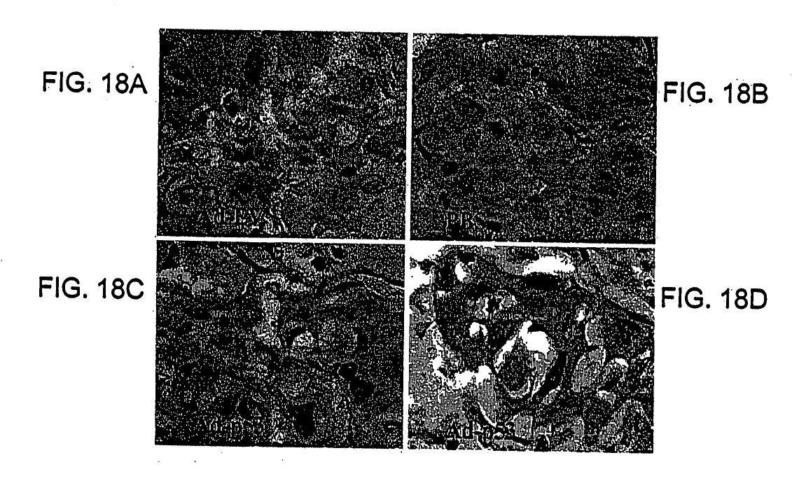

FIG. 19. Diagram of a suitable aerosol application unit employed for inhalation delivery of viral compositions to C3H mice.

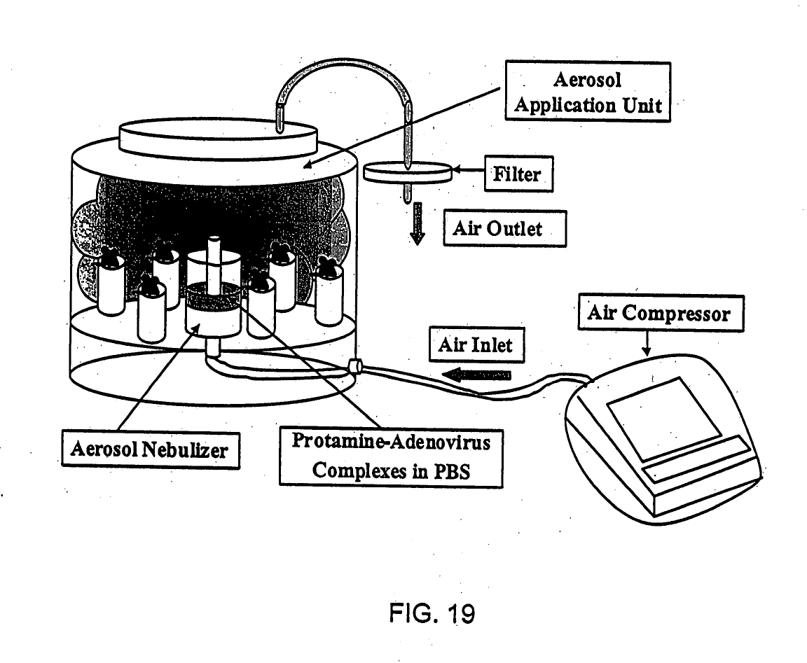

FIG. 20. Pulmonary expression of GFP 48 hours after delivery by inhalation to C3H mice of P-Ad-GFP. Photographs show different magnifications, 20× (A), 40× (B), and 100× (C).

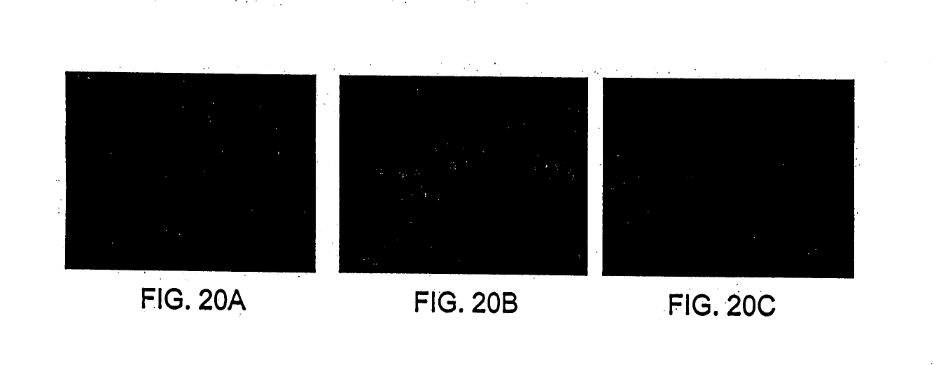

FIG. 21. Structures of protein conjugate compounds that may serve as a linking moiety.

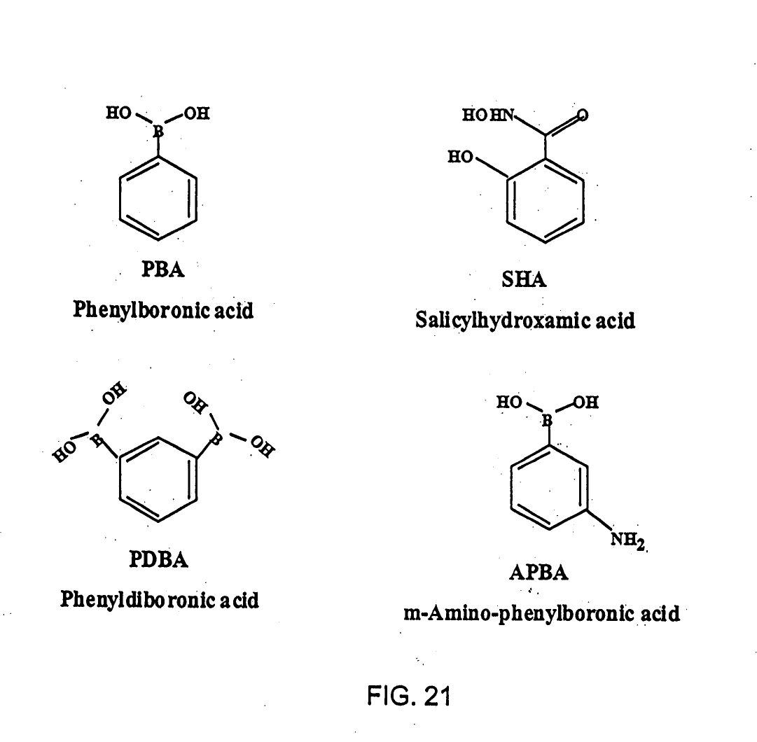

FIG. 22. Conjugation of a protamine-peptide by PBA-SHA linking chemistry.

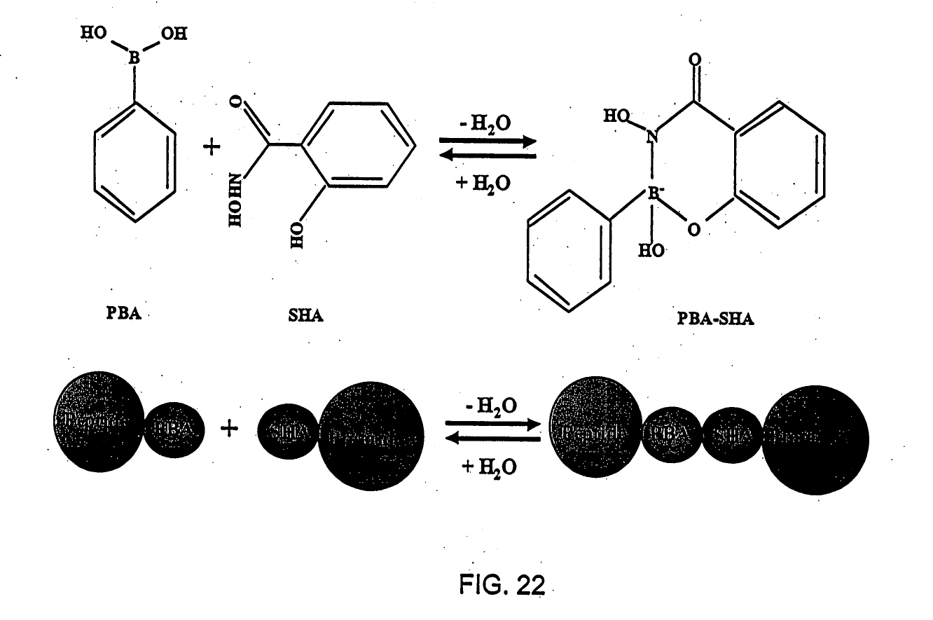

FIG. 23. Structure of protamine-uPA peptide complex using PDBA-SHA linking chemistry.

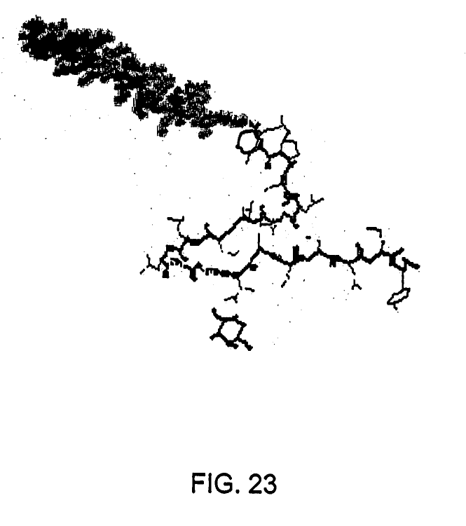

FIG. 24. Illustrates an example of the effects of systemic administration with P-Ad-p53 complexes on A549 metastases in nude mice.

### DESCRIPTION OF ILLUSTRATIVE EMBODIMENTS

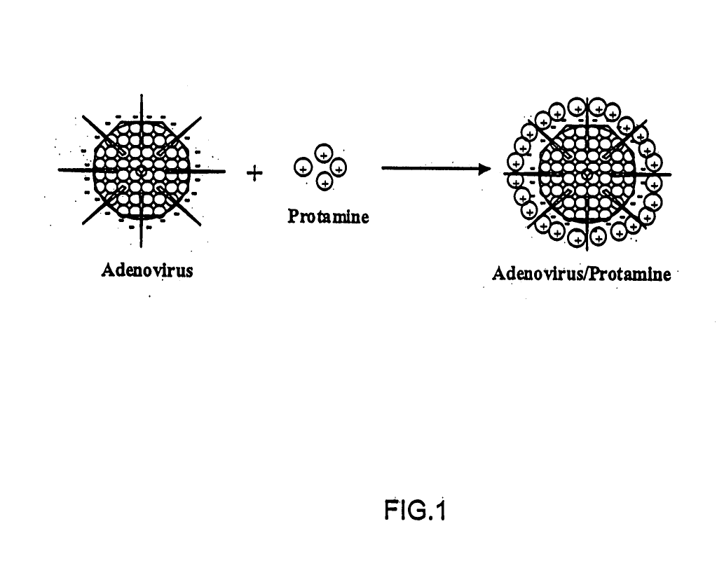

Embodiments of the invention include compositions and methods involving a viral composition comprising a protamine-viral vector complex that affects the growth and/or viability of a cancer cell. In certain embodiments, compositions are administered to treat and/or prevent a diseased condition, in particular lung cancer. The viral vector preferably comprises a polynucleotide, i.e., an expression vector, encoding a therapeutic gene, such as a tumor suppressor. Administration in vivo of the viral composition has demonstrated increased transduction efficiency, decreased viral vector-induced neutralizing antibody production and reduced viral vector-based toxicity as compared to viral vector compositions without protamine. Certain embodiments of the invention may allow administration of lower viral particle concentrations and fewer doses of viral compositions. Typically, the composition and methods do not induce hepatic toxicity in the patient or subject. Improved transduction of therapeutic viral vectors will bestow preventative and therapeutic benefits through the body's enhanced ability to prevent, inhibit, or reduce the incidence of infections, diseases, or other conditions. FIG. 1 illustrates an exemplary viral composition of the present invention.

Embodiments of the invention include a viral composition that provides high level expression of transgenes in the cells of the lung, tumor cells and metastasized tumor cells in vitro, ex vivo, or in vivo. The improvements described herein will be useful and advantageous over an adenovirus composition without protamine in allowing the application of much lower doses of adenovirus to achieve the same or improved efficacy, reducing adenovirus-induced cytotoxicity, and reducing costs associated with decreased adenoviral vector doses.

The viral composition may further comprise a targeting moiety, such as a peptide or a polypeptide. The targeting moiety is understood to enhance and/or improve delivery of the viral composition to a malignancy as compared to specificity of delivering the viral composition lacking the targeting moiety.

I. Protamine

The present invention provides methods of treating a malignancy by administering an effective amount of a viral composition comprising a viral vector and a protamine molecule. A skilled artisan is aware that protamine is a FDA-approved anti-heparin drug and recognizes that protamine is readily available from commercial manufacturers.

Administering the viral compositions of the present invention has led to high level expression of transgenes in vitro and in vivo, and, further, inhibited the development of metastases in vivo.

The effect of ionic charge on transduction efficiency in vitro has been investigated with respect to adenoviral vectors. Currently, the mechanisms for adenoviral transduction is believed to be mediated by interactions between adenoviral proteins and cell surface receptors and molecules such as integrins and CARs (Goldman et al., 1998; Goldman et al., 1995; Wichham et al., 1996). Although the mechanisms that control the initial interactions between adenoviruses and target cells are still unclear, accumulating evidence suggests that transfer efficiency is retarded or reduced by electrostatic repulsion between the negatively charged cell surface and the negatively charged adenoviral particles (Fasbender et al., 1997; Goldman et al., 1997; Lanuti et al., 1999; Li et al., 1997; Li et al., 1998). Studies have shown that either the removal of negatively charged molecules on cell surface in cultured epithelial cells or conjugation of polycations such as polybrene, poly-1-Lysine, DEAE-dextran, and protamine with adenoviral vectors facilitated the epithelial cell uptake of adenoviral particles and improved the efficiency of adenoviral vector-mediated gene transfer in vitro and in vivo (Fasbender et al., 1997; Lanuti et al., 1999; Arcasoy et al., 1997a and 1997b; Kaplan et al., 1997; Kaplan et al., 1998). However, these studies failed to show an increase in the therapeutic efficacy of the compositions. On the other hand, polyanions and heparins have completely abrogated the effects of polycations on the transduction efficiency (Arcasoy et al., 1997a and 1997b).

The present invention uses protamine, as a highly positively charged small peptide molecule, together with viral vectors, e.g., adenoviral particles, to enhance gene transfer and improve clinical efficacy. An increase in clinical efficacy may be due to various characteristics of the inventive compositions, including reduction of induced immunization against the viral vector and/or reduction of the viral vector-induced cytotoxicity.

Protamine coordinates the net negative charges on viral envelops, neutralizes cell surface negative charge, and facilitates attachment of the viral particles to the cell surface. Transduction of viral compositions of the present invention occurred with enhanced efficiency in vitro and in vivo. Further, transgene expression was markedly improved in vitro and in vivo. For example, administration of protamine-adenovirus complexes via intravenous injection efficiently delivered the viral compositions to lung cells and pulmonary metastases. The viral compositions effectively inhibited the development of metastases and metastases tumor growth in mice. Administration of the viral composition by intratumorally injection also enhanced the clinical efficacy of adenoviral compositions in representative animal models of human cancer. The respiratory inhalation of the aerosolized protamine-adenoviral vector complexes efficiently delivered adenoviral vectors to the lung bronchial epithelial cells and terminal lung cells. Unexpectedly, the systemic administration of the viral compositions also reduced cellular immune responses and hepatic toxicity that were otherwise induced by adenoviral vectors in vivo.

A skilled artisan is aware of sequence repositories, such as GenBank, to obtain nucleic acid and amino acid sequences utilized in the present invention. Examples of the organisms having a protamine molecule and respective amino acid sequences for the present invention include, but are not limited to, the following: Potorous longipes, gene accession no. AAG27965.1 (SEQ ID NO:7); Aepyprymnus rufescens, gene accession no. AAG27964.1 (SEQ ID NO:8); Bettongia penicillata, gene accession no. AAG27963.1 (SEQ ID NO:9); Hypsiprymnodon moschatus, gene accession no. AAG27962.1 (SEQ ID NO:10); Lagorchestes hirsutus, gene accession no. AAG27961.1 (SEQ ID NO:11); Onychogalea unguifera, gene accession no. AAG27960.1 (SEQ ID NO:12); Onychogalea fraenata, gene accession no. AAG27959.1 (SEQ ID NO:13); Setonix brachyurus, gene accession no. AAG27958.1 (SEQ ID NO:14); Dorcopsis veterum, gene accession no. AAG27957.1 (SEQ ID NO:15); Dorcopsulus vanheurni, gene accession no. AAG27956.1 (SEQ ID NO:16); Peradorcas concinna, gene accession no. AAG27955.1 (SEQ ID NO:17); Dendrolagus goodfellowi, gene accession no. AAG27954.1 (SEQ ID NO:18); Dendrolagus dorianus, gene accession no. AAG27953.1 (SEQ ID NO:19); Petrogale xanthopus, gene accession no. AAG27952.1 (SEQ ID NO:20); Thylogale stigmatica, gene accession no. AAG27951.1 (SEQ ID NO:21); Macropus parryi, gene accession no. AAG27950.1 (SEQ ID NO:22); Phascogale calura, gene accession no. AAC15630.1 (SEQ ID NO:23); Murexia rothschildi, gene accession no. AAC15629.1 (SEQ ID NO:24); Antechinus naso, gene accession no. AAC15628.1 (SEQ ID NO:25); Antechinus habbema, gene accession no. AAC15627.1 (SEQ ID NO:26); Oncorhynehus mykiss, gene accession No. X01204 (SEQ ID NO:27); and oncorhynchun keta, gene accession No. X07511 (SEQ ID NO:28). All gene accession numbers (GenBank Accession numbers) are hereby incorporated by reference in their entirety herein.

The present invention exploits the inventors' identification of a molecule that improves transduction efficiency and/or therapeutic efficacy in vivo and in vitro, as well as reduces viral vector-induced antibody production and cytotoxicity. Therefore, the viral compositions can be used to shuttle or transport preventative and therapeutic compounds or nucleic acids to a malignancy or pre-cancerous growth for the treatment of diseases, conditions, or disorders. Additionally, it is contemplated that the present invention includes the use of peptide sequences that mimic the coordinating activity of protamine to the vector such that these sequences can be used as the previously described delivery shuttle system. Examples of such are discussed later.

II. Viral Vectors and Gene Transfer

Some of the major shortcomings of vector-mediated gene therapy is the relative low efficiency of gene transfer to the target tissues and tumors in vivo, short-term expression of transgenes, and a diminishing of transgene expression after repeated administration. In particular, cellular immune-responses have been shown to reduce transgene expression from adenoviral expression vectors, thereby significantly limiting treatment efficacy. Improvements in transduction efficiency and expression of transgenes in vitro and in vivo will be useful and advantageous over viral vectors not complexed with protamine or a similar molecule.

Embodiments of the invention include viral compositions comprising adenoviral vectors having a polynucleotide encoding a tumor suppressor, and a protamine molecule. A number of proteins have been characterized as tumor suppressors, which define a class of proteins that are involved in the regulation of cell proliferation. The loss of wild-type tumor suppressor activity is associated with neoplastic or unregulated cell growth. It has been shown by several groups that the neoplastic growth of cells lacking a wild-type copy of a particular tumor suppressor can be halted by the addition of a wild-type version of that tumor suppressor.

The invention contemplates the use of a viral vector complexed to a protamine molecule for the delivery of a tumor suppressor, such as p53 (human sequence found in Lamb et al., 1986, hereby specifically incorporated by reference) (SEQ ID NO:1 is the nucleic acid sequence and SEQ ID NO:2 is the amino acid sequence). Other tumor suppressors that may be employed according to the present invention include p21, p15, BRCA1, BRCA2, IRF-1, PTEN (MMAC1), FHIT, MDA7, Rb, APC, DCC, NF-1, NF-2, WT-1, MEN-I, MEN-II, zac1, p73, VHL, FCC, and MCC. In preferred embodiments, the tumor suppressor is MDA7 (GenBank Accession # U16261) (SEQ ID NO:3 is the nucleic acid sequence and SEQ ID NO:4 is the amino acid sequence) or PTEN (SEQ ID NO:5 is the nucleic acid sequence and SEQ ID NO:6 is the amino acid sequence) (U.S. Patent Application No. 60/329,637, which is hereby incorporated by reference) or FHIT (GenBank Accession # NM_{—}002012) (SEQ ID NO:29 is the nucleic acid sequence and SEQ ID NO:30 is the amino acid sequence).

The gene transfer involved in the present invention is effected by a viral vector, and in specific embodiments, an adenoviral vector. A viral vector typically comprises a polynucleotide encoding a viral expression vector.

A. Viral Vectors

The methods and compositions described herein include adenoviral constructs; the methods and compositions described may be applicable to the construction of constructs using other viral vectors including but not limited to retroviruses, herpes viruses, adeno-associated viruses, vaccinia viruses. The discussion below provides details regarding the characteristics of each of these viruses in relation to their application in therapeutic compositions.

1. Adenovirus

Certain embodiments of the invention include the use of an adenovirus vector for the delivery a therapeutic gene and/or a therapeutic vector, e.g., an ADP overexpressing vector. A therapeutic gene may be provided by an expression cassette or an adenoviral expression vector. “Adenoviral expression vector,” as used herein, is meant to include those constructs containing adenovirus sequences sufficient to (a) support packaging of the construct and (b) to express a polynucleotide, a protein, and/or a polynucleotide (e.g., ribozyme or an mRNA) that has been cloned therein or provide a therapeutic benefit, e.g., overexpression of ADP. Expression may or may not require that a gene product, e.g. a protein, be synthesized. For exemplary methods and compositions related to adenovirus, adenoviral vectors and their derivatives see U.S. Pat. Nos. 6,511,847, 6,410,029, 6,410,010, 6,143,290, 6,110,744, 6,069,134, 6,017,524, 5,747,469, each of which is incorporated herein by reference.

An expression vector may comprise a genetically engineered form of adenovirus. Knowledge of the genetic organization of adenovirus, a 36 kb, linear, double-stranded DNA virus, allows substitution of pieces of adenoviral DNA with foreign sequences up to and greater than 7 kb (Grunhaus and Horwitz, 1992). In contrast to retroviruses, the adenoviral infection of host cells does not result in chromosomal integration because adenoviral DNA can replicate in an episomal manner without potential genotoxicity. As used herein, the term “genotoxicity” refers to permanent inheritable host cell genetic alteration. Also, adenoviruses are structurally stable, and no genome rearrangement has been detected after extensive amplification of normal derivatives. Adenovirus can infect virtually all epithelial cells regardless of their cell cycle stage.

One potential therapy under active investigation is treating tumors with recombinant viral vectors expressing anti-cancer therapeutic proteins. Adenovirus-based vectors contain several characteristics that make them conceptually appealing for use in treating cancer, as well as for therapy of genetic disorders. Adenoviruses (hereinafter used interchangeably with “Ads”) can easily be grown in culture to high titer stocks that are stable. They have a broad host range, replicating in most human cancer cell types. Their genome can be manipulated by site-directed mutation and insertion of foreign genes expressed from foreign promoters.

The adenovirion includes a DNA-protein core within a protein capsid (reviewed by Stewart et al., “Adenovirus structure by x-ray crystallography and electron microscopy.” in: The Molecular Repertoire of Adenoviruses, Doerfler, W. et al., (ed), Springer-Verlag, Heidelberg, Germany, p. 25-38). Virions bind to a specific cellular receptor, are endocytosed, and the genome is extruded from endosomes and transported to the nucleus. The genome is a linear double-stranded DNA of about 36 kbp, encoding about 36 genes. In the nucleus, the “immediate early” E1A proteins are expressed initially, and these proteins induce expression of the “delayed early” proteins encoded by the E1B, E2, E3, and E4 transcription units (reviewed by Shenk, T. “Adenoviridae: the viruses and their replication” in: Fields Virology, Fields, B. N. et al., Lippencott-Raven, Philadelphia, p. 2111-2148). E1A proteins also induce or repress cellular genes, resulting in stimulation of the cell cycle. About 23 early proteins function to usurp the host cell and initiate viral DNA replication. Cellular protein synthesis is shut off, and the cell becomes a factory for making viral proteins.

Virions assemble in the nucleus at about 1 day post infection (p.i.), and after 2-3 days the cell lyses and releases progeny virus. Cell lysis is mediated by the E3 11.6K protein, which has been renamed “adenovirus death protein” (ADP) (Tollefson et al., 1996a; Tollefson et al., 1996b). The term ADP as used herein in a generic sense refers collectively to ADP's from adenoviruses such as, e.g. Ad type 1 (Ad1), Ad type 2 (Ad2), Ad type 5 (Ad5) or Ad type 6 (Ad6) all of which express homologous ADP's with a high degree of sequence similarity.

The Ad vectors being investigated for use in anti-cancer and gene therapy are based on recombinant adenovirus that are either replication-defective or replication-competent. Typical replication-defective Ad vectors lack the E1A and E1B genes (collectively known as E1) and in some embodiments, contain in their place an expression cassette consisting of a promoter and pre-mRNA processing signals which drive expression of a foreign gene. (See e.g., Felzmann et al., 1997; Topf et al., 1998; Putzer et al., 1997; Arai et al., 1997, each of which is incorporated herein by reference). These vectors are unable to replicate because they lack the E1A genes required to induce Ad gene expression and DNA replication. In addition, the E3 genes are usually deleted because they are not essential for virus replication in cultured cells.

The adenoviral vector according to the invention may be engineered to be conditionally replicative (CRAd vectors) in order to replicate selectively in specific host cells (i.e. proliferative cells), for examples see Heise and Kim, 2000; Bischoff et al., 1996; Rodriguez et al., 1997; Alemany et al., 2000; Doronin et al., 2001; Suzuki et al., 2001, each of which is incorporated herein by reference. Conditionally replicative adenovirus (CRAd) vectors are designed for specific oncolytic replication in tumor tissues with concomitant sparing of normal cells. As such, conditionally replicative adenoviruses offer a level of anticancer potential for malignancies that have been refractory to previous cancer gene therapy interventions.

Several groups have proposed using replication-competent Ad vectors for therapeutic use. Replication-competent vectors retain Ad genes essential for replication and thus, do not require complementing cell lines to replicate. Replication-competent Ad vectors lyse cells as a natural part of the life cycle of the vector. Another advantage of replication-competent Ad vectors occurs when the vector is engineered to encode and express a foreign protein. (See e.g., Lubeck et al., 1994). Such vectors would be expected to greatly amplify synthesis of the encoded protein in vivo as the vector replicates. For use as anti-cancer agents, replication-competent viral vectors would theoretically also be advantageous in that they should replicate and spread throughout the tumor, not just in the initially infected cells as is the case with replication-defective vectors.

Certain embodiments include vectors which are replication competent in neoplastic cells. Replication of the virus may be engineered to (a) be restricted to neoplastic cells, e.g., by replacing the E4, or other adenoviral promoter with a tissue specific or tumor specific promoter and/or (b) lack expression of one or more of the E3 gp19K; RIDa; RIDb; and 14.7K proteins. In some embodiments, an anti-cancer product is inserted into the E3 or other adenoviral region.

Replication competent vectors may or may not overexpress an adenovirus death protein (ADP). The overexpression of ADP by a recombinant adenovirus allows the construction of a replication-competent adenovirus that kills neoplastic cells and spreads from cell-to-cell at a rate similar to or faster than that exhibited by adenoviruses expressing wild-type levels of ADP, even when the recombinant adenovirus contains a mutation that would otherwise reduce its replication rate in non-neoplastic cells. Naturally-occurring adenoviruses express ADP in low amounts from the E3 promoter at early stages of infection, and begin to make ADP in large amounts only at 24-30 h p.i., once virions have been assembled in the cell nucleus. It is contemplated that other non-adenoviral vectors can be used to deliver ADP's cell-killing activity to neoplastic cells, including other viral vectors and plasmid expression vectors. Exemplary methods and compositions related to ADP expressing viruses may be found in PCT application WO 01/04282, which is incorporated herein by reference.

Because many human tissues are permissive for Ad infection, a method may be devised to limit the replication of the virus to the target cells. To specifically target tumor cells, several research laboratories have manipulated the E1B and E1A regions of the adenovirus. For example, Onyx Pharmaceuticals recently reported on adenovirus-based anti-cancer vectors which are replication-deficient in non-neoplastic cells, but which exhibit a replication phenotype in neoplastic cells lacking functional p53 and/or retinoblastoma (pRB) tumor suppressor proteins (U.S. Pat. No. 5,677,178; Heise et al., 1997; Bischoff et al., 1996, each of which are incorporated herein by reference). This phenotype is reportedly accomplished by using recombinant adenoviruses containing a mutation in the E1B region that renders the encoded E1B-55K protein incapable of binding to p53 and/or a mutation (s) in the E1A region which make the encoded E1A protein (P289R or p243R) incapable of binding to pRB and/or p300 and/or p107. E1B-55K has at least two independent functions: it binds and inactivates the tumor suppressor protein p53, and it is required for efficient transport of Ad mRNA from the nucleus. Because these E1B and E1A viral proteins are involved in forcing cells into S-phase, which is required for replication of adenovirus DNA, and because the p53 and pRB proteins block cell cycle progression, the recombinant adenovirus vectors described by Onyx should replicate in cells defective in p53 and/or pRB, which is the case for many cancer cells, but not in cells with wild-type p53 and/or pRB. Onyx has reported that replication of an adenovirus lacking E1B-55K, named ONYX-015, was restricted to p53-minus cancer cell lines (Bischoff et al., supra), and that ONYX-015 slowed the growth or caused regression of a p53-minus human tumor growing in nude mice (Heise et al., supra). Others have challenged the Onyx report claiming that replication of ONYX-015 is independent of p53 genotype and occurs efficiently in some primary cultured human cells (Harada and Berk, 1999). ONYX-015 does not replicate as well as wild-type adenovirus because E1B-55K is not available to facilitate viral mRNA transport from the nucleus. Also, ONYX-015 expresses less ADP than wild-type virus.

As an extension of the ONYX-015 concept, a replication-competent adenovirus vector was designed that has the gene for E1B-55K replaced with the herpes simplex virus thymidine kinase gene (Wilder et al., 1999a). The group that constructed this vector reported that the combination of the vector plus gancyclovir showed a therapeutic effect on a human colon cancer in a nude mouse model (Wilder et al., 1999b). However, this vector lacks the gene for ADP, and accordingly, the vector will lyse cells and spread from cell-to-cell less efficiently than an equivalent vector that expresses ADP.

Thus, there is a continuing need for an efficient and effective delivery of various anti-cancer adenovirus vectors, in particular those viruses that can specifically target neoplastic cells, while replicating poorly or not at all in normal tissue, and efficiently spreading to neighboring neoplastic cells, thereby maximizing the cancer-killing ability of the adenovirus vector. For exemplary methods and compositions related to replicating adenoviruses see PCT application WO 02/24640 and Doronin et al., 2001, each of which are incorporated herein by reference.

Recombinant adenovirus may be generated, as is well known in the art, from homologous recombination between shuttle vector and provirus vector. Generation and propagation of the current adenovirus vectors may depend on a unique helper cell line designated 293, which was transformed from human embryonic kidney cells by Ad5 DNA fragments and constitutively expresses E1 proteins (Graham et al., 1977). Since the E3 region is dispensable from the adenovirus genome (Jones and Shenk, 1978), adenovirus vectors may carry foreign DNA in either the E1, the E3 or both regions (Graham and Prevec, 1991). In nature, adenovirus can package approximately 105% of the wild-type genome (Ghosh-Choudhury et al., 1987), providing capacity for about 2 extra kb of DNA.

Helper cell lines may be derived from human cells such as human embryonic kidney cells, muscle cells, hematopoietic cells or other human embryonic mesenchymal or epithelial cells. Alternatively, the helper cells may be derived from the cells of other mammalian species that are permissive for human adenovirus. Such cells include, e.g., Vero cells or other monkey embryonic mesenchymal or epithelial cells. As stated above, the preferred helper cell line is 293. In various embodiments a helper cell may not be needed.

Recently, Racher et al. (1995) disclosed improved methods for culturing 293 cells and propagating adenovirus. In one format, natural cell aggregates are grown by inoculating individual cells into 1 liter siliconized spinner flasks (Techne, Cambridge, UK) containing 100-200 ml of medium. Following stirring at 40 rpm, the cell viability is estimated with trypan blue. In another format, Fibra-Cel microcarriers (Bibby Sterlin, Stone, UK) (5 g/l) is employed as follows. A cell inoculum, resuspended in 5 ml of medium, is added to the carrier (50 ml) in a 250 ml Erlenmeyer flask and left stationary, with occasional agitation, for 1 to 4 h. The medium is then replaced with 50 ml of fresh medium and shaking is initiated. For virus production, cells are allowed to grow to about 80% confluence, after which time the medium is replaced (to 25% of the final volume) and adenovirus added at an MOI of 0.05. Cultures are left stationary overnight, following which the volume is increased to 100% and shaking commenced for another 72 h. Other exemplary methods for the production of adenovirus may be found in U.S. Pat. Nos. 6,194,191, 6,485,958, 6,040,174, 5,837,520, and the like, each of which is incorporated herein by reference.

The adenovirus vector may be replication defective (replication-deficient), replication competent, conditionally defective (conditionally-replicative), or replication-restricted. The nature of the adenovirus vector is not believed to be crucial to the successful practice of the invention. The adenovirus may be of any of the 42 different known stereotypes or subgroups A-F. Adenovirus type 5 of subgroup C is the preferred starting material in order to obtain an adenovirus vector for use in the present invention. This is because Adenovirus type 5 is a human adenovirus about which a great deal of biochemical, medical and genetic information is known, and it has historically been used for most constructions employing adenovirus as a vector.

As stated above, the typical vector according to the present invention may or may not be replication defective. Thus, in certain embodiments, the polynucleotide encoding the gene of interest may be introduced at the position from which the E1-coding sequences, or other adenoviral sequences have been removed. However, the position of insertion of the construct within the adenovirus sequences is not critical to the invention. The polynucleotide encoding the gene of interest may also be inserted in lieu of the deleted E3 region in E3 replacement vectors as described by Karlsson et al. (1986), or in the E4 region where a helper cell line or helper virus complements the E4 defect.

Adenovirus is easy to grow and manipulate and exhibits broad host range in vitro and in vivo. This group of viruses can be obtained in high titers, e.g., 10⁹-10¹¹ plaque-forming units per ml, and they are highly infective. The life cycle of adenovirus does not require integration into the host cell genome. The foreign genes delivered by adenovirus vectors are episomal and, therefore, have low genotoxicity to host cells. No side effects have been reported in studies of vaccination with wild-type adenovirus (Couch et al., 1963; Top et al., 1971), demonstrating their safety and therapeutic potential as in vivo gene transfer vectors.

Adenovirus vectors have been used in eukaryotic gene expression investigations (Levrero et al., 1991; Gomez-Foix et al., 1992) and vaccine development (Grunhaus and Horwitz, 1992; Graham and Prevec, 1992). Recently, animal studies suggested that recombinant adenovirus could be used for gene therapy (Stratford-Perricaudet and Perricaudet, 1991; Stratford-Perricaudet et al., 1990; Rich et al., 1993). Studies in administering recombinant adenovirus to different tissues include trachea instillation (Rosenfeld et al., 1991; Rosenfeld et al., 1992), muscle injection (Ragot et al., 1993), peripheral intravenous injections (Herz and Gerard, 1993), intranasal inoculation (Ginsberg et al., 1991), aerosol administration to lung (Bellon, 1996) intra-peritoneal administration (Song et al., 1997), Intra-pleural injection (Elshami et al., 1996) administration to the bladder using intra-vesicular administration (Werthman, et al., 1996), Subcutaneous injection including intraperitoneal, intrapleural, intramuscular or subcutaneously) (Ogawa, 1989) ventricular injection into myocardium (heart, French et al., 1994), liver perfusion (hepatic artery or portal vein, Shiraishi et al., 1997) and stereotactic inoculation into the brain (Le Gal La Salle et al., 1993).

2. Retrovirus

The retroviruses are a group of single-stranded RNA viruses characterized by an ability to convert their RNA to double-stranded DNA in infected cells by a process of reverse-transcription (Coffin, 1990). The resulting DNA then stably integrates into cellular chromosomes as a provirus and directs synthesis of viral proteins. The integration results in the retention of the viral gene sequences in the recipient cell and its descendants. The retroviral genome contains three genes, gag, pol, and env that code for capsid proteins, polymerase enzyme, and envelope components, respectively. A sequence found upstream from the gag gene contains a signal for packaging of the genome into virions. Two long terminal repeat (LTR) sequences are present at the 5′ and 3′ ends of the viral genome. These contain strong promoter and enhancer sequences and are also required for integration in the host cell genome (Coffin, 1990).

In order to construct a retroviral vector, a nucleic acid encoding a gene of interest is inserted into the viral genome in the place of certain viral sequences to produce a virus that is replication-defective. In order to produce virions, a packaging cell line containing the gag, pol, and env genes but without the LTR and packaging components is constructed (Mann et al., 1983). When a recombinant plasmid containing a CDNA, together with the retroviral LTR and packaging sequences is introduced into this cell line (by calcium phosphate precipitation for example), the packaging sequence allows the RNA transcript of the recombinant plasmid to be packaged into viral particles, which are then secreted into the culture media (Nicolas and Rubenstein, 1988; Temin, 1986; Mann et al., 1983). The media containing the recombinant retroviruses is then collected, optionally concentrated, and used for gene transfer. Retroviral vectors are able to infect a broad variety of cell types. However, integration and stable expression require the division of host cells (Paskind et al., 1975).

A novel approach designed to allow specific targeting of retrovirus vectors was recently developed based on the chemical modification of a retrovirus by the chemical addition of lactose residues to the viral envelope. This modification could permit the specific infection of hepatocytes via sialoglycoprotein receptors.

A different approach to targeting of recombinant retroviruses was designed in which biotinylated antibodies against a retroviral envelope protein and against a specific cell receptor were used. The antibodies were coupled via the biotin components by using streptavidin (Roux et al., 1989). Using antibodies against major histocompatibility complex class I and class II antigens, they demonstrated the infection of a variety of human cells that bore those surface antigens with an ecotropic virus in vitro (Roux et al., 1989).

There are certain limitations to the use of retrovirus vectors in all aspects of the present invention. For example, retrovirus vectors usually integrate into random sites in the cell genome. This can lead to insertional mutagenesis through the interruption of host genes or through the insertion of viral regulatory sequences that can interfere with the function of flanking genes (Varmus et al., 1981). Another concern with the use of defective retrovirus vectors is the potential appearance of wild-type replication-competent virus in the packaging cells. This can result from recombination events in which the intact- sequence from the recombinant virus inserts upstream from the gag, pol, env sequence integrated in the host cell genome. However, new packaging cell lines are now available that should greatly decrease the likelihood of recombination (Markowitz et al., 1988; Hersdorffer et al., 1990).

3. Herpesvirus

Because herpes simplex virus (HSV) is neurotropic, it has generated considerable interest in treating nervous system disorders. Moreover, the ability of HSV to establish latent infections in non-dividing neuronal cells without integrating in to the host cell chromosome or otherwise altering the host cell's metabolism, along with the existence of a promoter that is active during latency makes HSV an attractive vector. And though much attention has focused on the neurotropic applications of HSV, this vector also can be exploited for other tissues given its wide host range.

Another factor that makes HSV an attractive vector is the size and organization of the genome. Because HSV is large, incorporation of multiple genes or expression cassettes is less problematic than in other smaller viral systems. In addition, the availability of different viral control sequences with varying performance (temporal, strength, etc.) makes it possible to control expression to a greater extent than in other systems. It also is an advantage that the virus has relatively few spliced messages, further easing genetic manipulations.

HSV also is relatively easy to manipulate and can be grown to high titers. Thus, delivery is less of a problem, both in terms of volumes needed to attain sufficient MOI and in a lessened need for repeat dosings. For a review of HSV as a gene therapy vector, see Glorioso et al. (1995).

HSV, designated with subtypes 1 and 2, are enveloped viruses that are among the most common infectious agents encountered by humans, infecting millions of human subjects worldwide. The large, complex, double-stranded DNA genome encodes for dozens of different gene products, some of which derive from spliced transcripts. In addition to virion and envelope structural components, the virus encodes numerous other proteins including a protease, a ribonucleotides reductase, a DNA polymerase, a ssDNA binding protein, a helicase/primase, a DNA dependent ATPase, a dUTPase and others.

HSV genes form several groups whose expression is coordinately regulated and sequentially ordered in a cascade fashion (Honess and Roizman, 1974; Honess and Roizman 1975; Roizman and Sears, 1995). The expression of α genes, the first set of genes to be expressed after infection, is enhanced by the virion protein number 16, or α-transinducing factor (Post et al., 1981; Batterson and Roizman, 1983; Campbell, et al., 1983). The expression of β genes requires functional α gene products, most notably ICP4, which is encoded by the α4 gene (DeLuca et al., 1985). γ genes, a heterogeneous group of genes encoding largely virion structural proteins, require the onset of viral DNA synthesis for optimal expression (Holland et al., 1980).

In line with the complexity of the genome, the life cycle of HSV is quite involved. In addition to the lytic cycle, which results in synthesis of virus particles and, eventually, cell death, the virus has the capability to enter a latent state in which the genome is maintained in neural ganglia until some as of yet undefined signal triggers a recurrence of the lytic cycle. Avirulent variants of HSV have been developed and are readily available for use in gene therapy contexts (U.S. Pat. No. 5,672,344).

4. Adeno-Associated Virus

Recently, adeno-associated virus (AAV) has emerged as a potential alternative to the more commonly used retroviral and adenoviral vectors. While studies with retroviral and adenoviral mediated gene transfer raise concerns over potential oncogenic properties of the former, and immunogenic problems associated with the latter, AAV has not been associated with any such pathological indications.

In addition, AAV possesses several unique features that make it more desirable than the other vectors. Unlike retroviruses, AAV can infect non-dividing cells; wild-type AAV has been characterized by integration, in a site-specific manner, into chromosome 19 of human cells (Kotin and Berns, 1989; Kotin et al., 1990; Kotin et al., 1991; Samulski et al., 1991); and AAV also possesses anti-oncogenic properties (Ostrove et al., 1981; Berns and Giraud, 1996). Recombinant AAV genomes are constructed by molecularly cloning DNA sequences of interest between the AAV ITRs, eliminating the entire coding sequences of the wild-type AAV genome. The AAV vectors thus produced lack any of the coding sequences of wild-type AAV, yet retain the property of stable chromosomal integration and expression of the recombinant genes upon transduction both in vitro and in vivo (Berns, 1990; Berns and Bohensky, 1987; Bertran et al., 1996; Kearns et al., 1996; Ponnazhagan et al., 1997a). Until recently, AAV was believed to infect almost all cell types, and even cross species barriers. However, it now has been determined that AAV infection is receptor-mediated (Ponnazhagan et al., 1996; Mizukami et al, 1996).

AAV utilizes a linear, single-stranded DNA of about 4700 base pairs. Inverted terminal repeats flank the genome. Two genes are present within the genome, giving rise to a number of distinct gene products. The first, the cap gene, produces three different virion proteins (VP), designated VP-1, VP-2 and VP-3. The second, the rep gene, encodes four non-structural proteins (NS). One or more of these rep gene products is responsible for transactivating AAV transcription. The sequence of AAV is provided by Srivastava et al., (1983) and in U.S. Pat. No. 5,252,479 (entire text of which is specifically incorporated herein by reference).

The three promoters in AAV are designated by their location, in map units, in the genome. These are, from left to right, p5, p19 and p40. Transcription gives rise to six transcripts, two initiated at each of three promoters, with one of each pair being spliced. The splice site, derived from map units 42-46, is the same for each transcript. The four non-structural proteins apparently are derived from the longer of the transcripts, and three virion proteins all arise from the smallest transcript.

AAV is not associated with any pathologic state in humans. Interestingly, for efficient replication, AAV requires “helping” functions from viruses such as herpes simplex virus I and II, cytomegalovirus, pseudorabies virus and, of course, adenovirus. The best characterized of the helpers is adenovirus, and many “early” functions for this virus have been shown to assist with AAV replication. Low level expression of AAV rep proteins is believed to hold AAV structural expression in check, and helper virus infection is thought to remove this block.

5. Vaccinia Virus

Vaccinia virus vectors have been used extensively because of the ease of their construction, relatively high levels of expression obtained, wide host range and large capacity for carrying DNA. Vaccinia contains a linear, double-stranded DNA genome of about 186 kb that exhibits a marked “A-T” preference. Inverted terminal repeats of about 10.5 kb flank the genome. The majority of essential genes appear to map within the central region, which is most highly conserved among poxviruses. Estimated open reading frames in vaccinia virus number from 150 to 200. Although both strands are coding, extensive overlap of reading frames is not common.

At least 25 kb can be inserted into the vaccinia virus genome (Smith and Moss, 1983). Prototypical vaccinia vectors contain transgenes inserted into the viral thymidine kinase gene via homologous recombination. Vectors are selected on the basis of a tk-phenotype. Inclusion of the untranslated leader sequence of encephalomyocarditis virus, the level of expression is higher than that of conventional vectors, with the transgenes accumulating at 10% or more of the infected cell's protein in 24 h (Elroy-Stein et al., 1989).

B. Regulatory Elements

The recombinant DNA techniques encompassed by the present invention to prepare and produce viral compositions including compositions comprising polynucleotide encoding a tumor suppressor may utilize recombinant vectors or expression constructs containing regulatory elements. These regulatory elements can include promoters (tissue-specific, non-tissue-specific, and inducible) and enhancers, polyadenylation sequences, and internal ribosomal entry sites (IRES).

1. Promoters

The nucleic acid encoding a gene product is under transcriptional control of a promoter. A “promoter” refers to a DNA sequence recognized by the synthetic machinery of the cell, or introduced synthetic machinery, required to initiate the specific transcription of a gene. The phrase “under transcriptional control” means that the promoter is in the correct location and orientation in relation to the nucleic acid to control RNA polymerase initiation and expression of the gene.

The term promoter will be used here to refer to a group of transcriptional control modules that are clustered around the initiation site for RNA polymerase II. Much of the thinking about how promoters are organized derives from analyses of several viral promoters, including those for the HSV thymidine kinase (tk) and SV40 early transcription units.

At least one module in each promoter functions to position the start site for RNA synthesis. The best known example of this is the TATA box, but in some promoters lacking a TATA box, such as the promoter for the mammalian terminal deoxynucleotidyl transferase gene and the promoter for the SV40 late genes, a discrete element overlying the start site itself helps to fix the place of initiation.

Additional promoter elements regulate the frequency of transcriptional initiation. The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either co-operatively or independently to activate transcription.

The particular promoter employed to control the expression of a nucleic acid sequence of interest is not believed to be important, so long as it is capable of directing the expression of the nucleic acid in the targeted cell. Thus, where a human cell is targeted, it is preferable to position the nucleic acid coding region adjacent to and under the control of a promoter that is capable of being expressed in a human cell. Generally speaking, such a promoter might include either a human or viral promoter.

In various embodiments, the human cytomegalovirus (CMV) immediate early gene promoter, the SV40 early promoter, the Rous sarcoma virus long terminal repeat, β-actin, rat insulin promoter and glyceraldehyde-3-phosphate dehydrogenase can be used to obtain high-level expression of the coding sequence of interest. The use of other viral or mammalian cellular or bacterial phage promoters which are well-known in the art to achieve expression of a coding sequence of interest is contemplated as well, provided that the levels of expression are sufficient for a given purpose. By employing a promoter with well-known properties, the level and pattern of expression of the protein of interest following transfection or transformation can be optimized.

Selection of a promoter that is regulated in response to specific physiologic or synthetic signals can permit inducible expression of the gene product. For example in the case where expression of a transgene, or transgenes when a multicistronic vector is utilized, is toxic to the cells in which the vector is produced in, it may be desirable to prohibit or reduce expression of one or more of the transgenes. Examples of transgenes that may be toxic to the producer cell line are pro-apoptotic and cytokine genes. Several inducible promoter systems are available for production of viral vectors where the transgene product may be toxic.

In some circumstances, it may be desirable to regulate expression of a transgene in a gene therapy vector. For example, different viral promoters with varying strengths of activity may be utilized depending on the level of expression desired. In mammalian cells, the CMV immediate early promoter if often used to provide strong transcriptional activation. Modified versions of the CMV promoter that are less potent have also been used when reduced levels of expression of the transgene are desired. When expression of a transgene in hematopoetic cells is desired, retroviral promoters such as the LTRs from MLV or MMTV are often used. Other viral promoters that may be used depending on the desired effect include SV40, RSV LTR, HIV-1 and HIV-2 LTR, adenovirus promoters such as from the E1A, E2A, or MLP region, AAV LTR, cauliflower mosaic virus, HSV-TK, and avian sarcoma virus.

Similarly tissue specific promoters may be used to effect transcription in specific tissues or cells so as to reduce potential toxicity or undesirable effects to non-targeted tissues. In the present invention, embodiments cover promoters that direct expression in epithelium cells, particularly mucosal epithelium. Endothelial-specific promoters direct the regulation of genes such as E-selectin, von Willebrand factor, TIE (Korhonen et al., 1995) and KDR/flk-1.

In certain indications, it may be desirable to activate transcription at specific times after administration of the gene therapy vector. This may be done with such promoters as those that are hormone or cytokine regulatable. For example in gene therapy applications where the indication is a gonadal tissue where specific steroids are produced or routed to, use of androgen or estrogen regulated promoters may be advantageous. Such promoters that are hormone regulatable include MMTV, MT-1, ecdysone and RuBisco. Other hormone regulated promoters such as those responsive to thyroid, pituitary and adrenal hormones are expected to be useful in the present invention. Cytokine and inflammatory protein responsive promoters that could be used include K and T Kininogen (Kageyama et al., 1987), c-fos, TNF-alpha, C-reactive protein (Arcone et al., 1988), haptoglobin (Oliviero et al., 1987), serum amyloid A2, C/EBP alpha, IL-1, IL-6 (Poli and Cortese, 1989), Complement C3 (Wilson et al., 1990), IL-8, alpha-1 acid glycoprotein (Prowse and Baumann, 1988), alpha-1 antitypsin, lipoprotein lipase (Zechner et al., 1988), angiotensinogen (Ron et al., 1991), fibrinogen, c-jun (inducible by phorbol esters, TNF-alpha, UV radiation, retinoic acid, and hydrogen peroxide), collagenase (induced by phorbol esters and retinoic acid), metallothionein (heavy metal and glucocorticoid inducible), Stromelysin (inducible by phorbol ester, interleukin-1 and EGF), alpha-2 macroglobulin and alpha-1 antichymotrypsin.

It is envisioned that cell cycle regulatable promoters may be useful in the present invention. For example, in a bi-cistronic gene therapy vector, use of a strong CMV promoter to drive expression of a first gene such as pl6 that arrests cells in the G1 phase could be followed by expression of a second gene such as p53 under the control of a promoter that is active in the G1 phase of the cell cycle, thus providing a “second hit” that would push the cell into apoptosis. Other promoters such as those of various cyclins, PCNA, galectin-3, E2F1, p53 and BRCA1 could be used.

Tumor specific promoters such as osteocalcin, hypoxia-responsive element (HRE), MAGE-4, CEA, alpha-fetoprotein, GRP78/BiP and tyrosinase may also be used to regulate gene expression in tumor cells. Other promoters that could be used according to the present invention include Lac-regulatable, chemotherapy inducible (e.g. MDR), and heat (hyperthermia) inducible promoters, radiation-inducible (e.g., EGR (Joki et al., 1995)), Alpha-inhibin, RNA pol III tRNA met and other amino acid promoters, U1 snRNA (Bartlett et al., 1996), MC-1, PGK, β-actin and α-globin. Many other promoters that may be useful are listed in Walther and Stein (1996).

It is envisioned that any of the above promoters alone or in combination with another may be useful according to the present invention depending on the action desired. In addition, this list of promoters is should not be construed to be exhaustive or limiting, those of skill in the art will know of other promoters that may be used in conjunction with the promoters and methods disclosed herein.

2. Enhancers

Enhancers are genetic elements that increase transcription from a promoter located at a distant position on the same molecule of DNA. Enhancers are organized much like promoters. That is, they are composed of many individual elements, each of which binds to one or more transcriptional proteins. The basic distinction between enhancers and promoters is operational. An enhancer region as a whole must be able to stimulate transcription at a distance; this need not be true of a promoter region or its component elements. On the other hand, a promoter must have one or more elements that direct initiation of RNA synthesis at a particular site and in a particular orientation, whereas enhancers lack these specificities. Promoters and enhancers are often overlapping and contiguous, often seeming to have a very similar modular organization.

3. Polyadenylation Signals

Where a cDNA insert is employed, one will typically desire to include a polyadenylation signal to effect proper polyadenylation of the gene transcript. The nature of the polyadenylation signal is not believed to be crucial to the successful practice of the invention, and any such sequence may be employed such as human or bovine growth hormone and SV40 polyadenylation signals. Also contemplated as an element of the expression cassette is a terminator. These elements can serve to enhance message levels and to minimize read through from the cassette into other sequences.

4. IRES

In certain embodiments of the invention, the use of internal ribosome entry site (IRES) elements is contemplated to create multigene, or polycistronic, messages. IRES elements are able to bypass the ribosome scanning model of 5′ methylated Cap dependent translation and begin translation at internal sites (Pelletier and Sonenberg, 1988). IRES elements from two members of the picornavirus family (poliovirus and encephalomyocarditis) have been described (Pelletier and Sonenberg, 1988), as well an IRES from a mammalian message (Macejak and Sarnow, 1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message.

Any heterologous open reading frame can be linked to IRES elements. This includes genes for secreted proteins, multi-subunit proteins, encoded by independent genes, intracellular or membrane-bound proteins and selectable markers. In this way, expression of several proteins can be simultaneously engineered into a cell with a single construct and a single selectable marker.

III. Nucleic Acids

Certain aspects of the present invention concern polynucleotides and/or nucleic acids, including polynucleotides and/or nucleic acids encoding tumor suppressors and viral expression vectors. In certain aspects, the nucleic acid of the present invention is directed to a nucleic acid encoding a tumor suppressor comprising a nucleic acid encoding a wild-type or mutant tumor suppressor. The nucleic acid encoding a tumor suppressor encodes at least one transcribed nucleic acid. The nucleic acid encoding a tumor suppressor may encodes at least one tumor suppressor protein, polypeptide or peptide, or biologically functional equivalent thereof. In other aspects, the nucleic acid comprises at least one nucleic acid segment of SEQ ID NO:1, SEQ ID NO:3, SEQ ID NO:5, or SEQ ID NO:29 or at least one biologically functional equivalent thereof.

The invention also concerns the isolation or creation of at least one recombinant construct, e.g., an expression construct, or at least one recombinant host cell through the application of recombinant nucleic acid technology known to those of skill in the art or as described herein. The recombinant construct or host cell may comprise at least one nucleic acid encoding a tumor suppressor, and may express at least one tumor suppressor protein, peptide or peptide, or at least one biologically functional equivalent thereof.

As used herein “wild-type” refers to the naturally occurring sequence of a nucleic acid at a genetic locus in the genome of an organism that encodes a functional, non-disease associated, gene product, and sequences transcribed or translated from such a nucleic acid. Thus, the term “wild-type” also may refer to the amino acid sequence encoded by the nucleic acid. As a genetic locus may have more than one sequence or alleles in a population of individuals, the term “wild-type” encompasses all such naturally occurring alleles. As used herein the term “polymorphic” means that variation exists (i.e. two or more alleles exist) at a genetic locus in the individuals of a population. As used herein “mutant” refers to a change in the sequence of a nucleic acid or its encoded protein, polypeptide or peptide that is the result of the hand of man.

A nucleic acid may be made by any technique known to one of ordinary skill in the art. Non-limiting examples of synthetic nucleic acid, particularly a synthetic oligonucleotide, include a nucleic acid made by in vitro chemically synthesis using phosphotriester, phosphite or phosphoramidite chemistry and solid phase techniques such as described in EP 266,032, incorporated herein by reference, or via deoxynucleoside H-phosphonate intermediates as described by Froehler et al, 1986, and U.S. Pat. No. 5,705,629, each incorporated herein by reference. A non-limiting example of enzymatically produced nucleic acid include one produced by enzymes in amplification reactions such as PCR™ (see for example, U.S. Pat. No. 4,683,202 and U.S. Pat. No. 4,682,195, each incorporated herein by reference), or the synthesis of oligonucleotides described in U.S. Pat. No. 5,645,897, incorporated herein by reference. A non-limiting example of a biologically produced nucleic acid includes recombinant nucleic acid production in living cells, such as recombinant DNA vector production in bacteria (see for example, Sambrook et al. 1989, incorporated herein by reference).

A nucleic acid may be purified on polyacrylamide gels, cesium chloride centrifugation gradients, or by any other means known to one of ordinary skill in the art (see for example, Sambrook et al. 1989, incorporated herein by reference).

The term “nucleic acid” or “polynucleotide” will generally refer to at least one molecule or strand of DNA, RNA or a derivative or mimic thereof, comprising at least one nucleobase, such as, for example, a naturally occurring purine or pyrimidine base found in DNA (e.g. adenine “A,” guanine “G,” thyrnine “T” and cytosine “C”) or RNA (e.g. A, G, uracil “U” and C). The term “nucleic acid” encompass the terms “oligonucleotide” and “polynucleotide.” The term “oligonucleotide” refers to at least one molecule of between about 3 and about 100 nucleobases in length. The term “polynucleotide” refers to at least one molecule of greater than about 100 nucleobases in length. These definitions generally refer to at least one single-stranded molecule, but in specific embodiments will also encompass at least one additional strand that is partially, substantially or fully complementary to the at least one single-stranded molecule. Thus, a nucleic acid may encompass at least one double-stranded molecule or at least one triple-stranded molecule that comprises one or more complementary strand(s) or “complement(s)” of a particular sequence comprising a strand of the molecule. As used herein, a single stranded nucleic acid may be denoted by the prefix “ss”, a double stranded nucleic acid by the prefix “ds”, and a triple stranded nucleic acid by the prefix

Thus, the invention also encompasses at least one nucleic acid that is complementary to a nucleic acid encoding a tumor suppressor. In particular embodiments the invention encompasses at least one nucleic acid or nucleic acid segment complementary to the sequence set forth in SEQ ID NO:1, SEQ ID NO:3, SEQ ID NO:5, or SEQ ID NO:29. As used herein, SEQ ID NO:1 refers to a polynucleotide sequence encoding p53, and a representative sequence of which can be found in gene accession no. HUMP53A11, open reading frame 1376 to 2554. As used herein, SEQ ID NO:3 refers to a polynucleotide sequence encoding MDA7, and a representative sequence of which is gene accession no. U16261. As used herein, SEQ ID NO:5 refers to a polynucleotide sequence encoding PTEN, and a representative sequence of which can is gene accession no. HSU93051. As used herein, SEQ ID NO:29 refers to a polynucleotide sequence encoding FHIT, a representative sequence of which is gene accession no. U46922.

Nucleic acid(s) that are “complementary” or “complement(s)” are those that are capable of base-pairing according to the standard Watson-Crick, Hoogsteen or reverse Hoogsteen binding complementarity rules. As used herein, the term “complementary” or “complement(s)” also refers to nucleic acid(s) that are substantially complementary, as may be assessed by the same nucleotide comparison set forth above. The term “substantially complementary” refers to a nucleic acid comprising at least one sequence of consecutive nucleobases, or semiconsecutive nucleobases if one or more nucleobase moieties are not present in the molecule, are capable of hybridizing to at least one nucleic acid strand or duplex even if less than all nucleobases do not base pair with a counterpart nucleobase.

In certain embodiments, a “substantially complementary” nucleic acid contains at least one sequence in which about 70%, about 71%, about 72%, about 73%, about 74%, about 75%, about 76%, about 77%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, to about 100%, and any range therein, of the nucleobase sequence is capable of base-pairing with at least one single or double stranded nucleic acid molecule during hybridization. In certain embodiments, the term “substantially complementary” refers to at least one nucleic acid that may hybridize to at least one nucleic acid strand or duplex in stringent conditions. In certain embodiments, a “partly complementary” nucleic acid comprises at least one sequence that may hybridize in low stringency conditions to at least one single or double stranded nucleic acid, or contains at least one sequence in which less than about 70% of the nucleobase sequence is capable of base-pairing with at least one single or double stranded nucleic acid molecule during hybridization.

As used herein, “hybridization”, “hybridizes” or “capable of hybridizing” is understood to mean the forming of a double or triple stranded molecule or a molecule with partial double or triple stranded nature. The term “hybridization”, “hybridize(s)” or “capable of hybridizing” encompasses the terms “stringent condition(s)” or “high stringency” and the terms “low stringency” or “low stringency condition(s).”

As used herein “stringent condition(s)” or “high stringency” are those that allow hybridization between or within one or more nucleic acid strand(s) containing complementary sequence(s), but precludes hybridization of random sequences. Stringent conditions tolerate little, if any, mismatch between a nucleic acid and a target strand. Such conditions are well known to those of ordinary skill in the art, and are preferred for applications requiring high selectivity. Non-limiting applications include isolating at least one nucleic acid, such as a gene or nucleic acid segment thereof, or detecting at least one specific mRNA transcript or nucleic acid segment thereof, and the like.

Stringent conditions may comprise low salt and/or high temperature conditions, such as provided by about 0.02 M to about 0.15 M NaCl at temperatures of about 50° C. to about 70° C. It is understood that the temperature and ionic strength of a desired stringency are determined in part by the length of the particular nucleic acid(s), the length and nucleobase content of the target sequence(s), the charge composition of the nucleic acid(s), and to the presence of formamide, tetramethylammonium chloride or other solvent(s) in the hybridization mixture. It is generally appreciated that conditions may be rendered more stringent, such as, for example, the addition of increasing amounts of formamide.

It is also understood that these ranges, compositions and conditions for hybridization are mentioned by way of non-limiting example only, and that the desired stringency for a particular hybridization reaction is often determined empirically by comparison to one or more positive or negative controls. Depending on the application envisioned it is preferred to employ varying conditions of hybridization to achieve varying degrees of selectivity of the nucleic acid(s) towards target sequence(s). In a non-limiting example, identification or isolation of related target nucleic acid(s) that do not hybridize to a nucleic acid under stringent conditions may be achieved by hybridization at low temperature and/or high ionic strength. Such conditions are termed “low stringency” or “low stringency conditions”, and non-limiting examples of low stringency include hybridization performed at about 0.15 M to about 0.9 M NaCl at a temperature range of about 20° C. to about 50° C. Of course, it is within the skill of one in the art to further modify the low or high stringency conditions to suite a particular application.

One or more nucleic acid(s) may comprise, or be composed entirely of, at least one derivative or mimic of at least one nucleobase, a nucleobase linker moiety and/or backbone moiety that may be present in a naturally occurring nucleic acid. As used herein a “derivative” refers to a chemically modified or altered form of a naturally occurring molecule, while the terms “mimic” or “analog” refers to a molecule that may or may not structurally resemble a naturally occurring molecule, but functions similarly to the naturally occurring molecule. As used herein, a “moiety” generally refers to a smaller chemical or molecular component of a larger chemical or molecular structure, and is encompassed by the term “molecule.”

As used herein a “nucleobase” refers to a naturally occurring heterocyclic base, such as A, T, G, C or U (“naturally occurring nucleobase(s)”), found in at least one naturally occurring nucleic acid (i.e. DNA and RNA), and their naturally or non-naturally occurring derivatives and mimics. Non-limiting examples of nucleobases include purines and pyrimidines, as well as derivatives and mimics thereof, which generally can form one or more hydrogen bonds (“anneal” or “hybridize”) with at least one naturally occurring nucleobase in manner that may substitute for naturally occurring nucleobase pairing (e.g. the hydrogen bonding between A and T, G and C, and A and U).

Nucleobase, nucleoside and nucleotide mimics or derivatives are well known in the art, and have been described in exemplary references such as, for example, Scheit, Nucleotide Analogs (John Wiley, New York, 1980), incorporated herein by reference.

As used herein, “nucleoside” refers to an individual chemical unit comprising a nucleobase covalently attached to a nucleobase linker moiety. A non-limiting example of a “nucleobase linker moiety” is a sugar comprising 5-carbon atoms (a “5-carbon sugar”), including but not limited to deoxyribose, ribose or arabinose, and derivatives or mimics of 5-carbon sugars. Non-limiting examples of derivatives or mimics of 5-carbon sugars include 2′-fluoro-2′-deoxyribose or carbocyclic sugars where a carbon is substituted for the oxygen atom in the sugar ring. By way of non-limiting example, nucleosides comprising purine (i.e. A and G) or 7-deazapurine nucleobases typically covalently attach the 9 position of the purine or 7-deazapurine to the 1′-position of a 5-carbon sugar. In another non-limiting example, nucleosides comprising pyrimidine nucleobases (i.e. C, T or U) typically covalently attach the 1 position of the pyrimidine to 1′-position of a 5-carbon sugar (Kornberg and Baker, DNA Replication, 2nd Ed. (Freeman, San Francisco, 1992). However, other types of covalent attachments of a nucleobase to a nucleobase linker moiety are known in the art, and non-limiting examples are described herein.

As used herein, a “nucleotide” refers to a nucleoside further comprising a “backbone moiety” generally used for the covalent attachment of one or more nucleotides to another molecule or to each other to form one or more nucleic acids. The “backbone moiety” in naturally occurring nucleotides typically comprises a phosphorus moiety, which is covalently attached to a 5-carbon sugar. The attachment of the backbone moiety typically occurs at either the 3′- or 5′-position of the 5-carbon sugar. However, other types of attachments are known in the art, particularly when the nucleotide comprises derivatives or mimics of a naturally occurring 5-carbon sugar or phosphorus moiety, and non-limiting examples are described herein.

In certain aspect, the present invention concerns at least one nucleic acid that is an isolated nucleic acid. As used herein, the term “isolated nucleic acid” refers to at least one nucleic acid molecule that has been isolated free of, or is otherwise free of, the bulk of the total genomic and transcribed nucleic acids of one or more cells, particularly mammalian cells, and more particularly malignant cells. In certain embodiments, “isolated nucleic acid” refers to a nucleic acid that has been isolated free of, or is otherwise free of, bulk of cellular components and macromolecules such as lipids, proteins, small biological molecules, and the like. As different species may have a RNA or a DNA containing genome, the term “isolated nucleic acid” encompasses both the terms “isolated DNA” and “isolated RNA”. Thus, the isolated nucleic acid may comprise a RNA or DNA molecule isolated from, or otherwise free of, the bulk of total RNA, DNA or other nucleic acids of a particular species. As used herein, an isolated nucleic acid isolated from a particular species is referred to as a “species specific nucleic acid.” When designating a nucleic acid isolated from a particular species, such as human, such a type of nucleic acid may be identified by the name of the species. For example, a nucleic acid isolated from one or more humans would be an “isolated human nucleic acid”.

Of course, more than one copy of an isolated nucleic acid may be isolated from biological material, or produced in vitro, using standard techniques that are known to those of skill in the art. In particular embodiments, the isolated nucleic acid is capable of expressing a protein, polypeptide or peptide that has a tumor suppressor activity. In other embodiments, the isolated nucleic acid comprises an isolated tumor suppressor gene.

Herein certain embodiments, a “gene” refers to a nucleic acid that is transcribed. As used herein, a “gene segment” is a nucleic acid segment of a gene. In certain aspects, the gene includes regulatory sequences involved in transcription, or message production or composition. In particular embodiments, the gene comprises transcribed sequences that encode for a protein, polypeptide or peptide. In other particular aspects, the gene comprises a nucleic acid encoding a tumor suppressor, and/or encodes a tumor suppressor polypeptide or peptide coding sequences. In keeping with the terminology described herein, an “isolated gene” may comprise transcribed nucleic acid(s), regulatory sequences, coding sequences, or the like, isolated substantially away from other such sequences, such as other naturally occurring genes, regulatory sequences, polypeptide or peptide encoding sequences, etc. In this respect, the term “gene” is used for simplicity to refer to a nucleic acid comprising a nucleotide sequence that is transcribed, and the complement thereof. In particular aspects, the transcribed nucleotide sequence comprises at least one functional protein, polypeptide and/or peptide encoding unit. As will be understood by those in the art, this function term “gene” includes both genomic sequences, RNA or cDNA sequences or smaller engineered nucleic acid segments, including nucleic acid segments of a non-transcribed part of a gene, including but not limited to the non-transcribed promoter or enhancer regions of a gene. Smaller engineered gene nucleic acid segments may express, or may be adapted to express using nucleic acid manipulation technology, proteins, polypeptides, domains, peptides, fusion proteins, mutants and/or such like. “Isolated substantially away from other coding sequences” means that the gene of interest, in this case the tumor suppressor gene(s), forms the significant part of the coding region of the nucleic acid, or that the nucleic acid does not contain large portions of naturally-occurring coding nucleic acids, such as large chromosomal fragments, other functional genes, RNA or CDNA coding regions. Of course, this refers to the nucleic acid as originally isolated, and does not exclude genes or coding regions later added to the nucleic acid by the hand of man.

In certain embodiments, the nucleic acid is a nucleic acid segment. As used herein, the term “nucleic acid segment”, are smaller fragments of a nucleic acid, such as for non-limiting example, those that encode only part of the tumor suppressor peptide or polypeptide sequence. Thus, a “nucleic acid segment” may comprise any part of the tumor suppressor gene sequence(s), of from about two nucleotides to the full length of the tumor suppressor peptide or polypeptide encoding region. In certain embodiments, the “nucleic acid segment” encompasses the full length tumor suppressor gene(s) sequence. In particular embodiments, the nucleic acid comprises any part of the SEQ ID NO:1 and/or SEQ ID NO:2 and/or SEQ ID NO:3 and/or SEQ ID NO:29 sequence(s), of from about 2 nucleotides to the full length of the sequence disclosed in SEQ ID NO:1 and/or SEQ ID NO:2 and/or SEQ ID NO:3 and/or SEQ ID NO:29.

Various nucleic acid segments may be designed based on a particular nucleic acid sequence, and may be of any length. By assigning numeric values to a sequence, for example, the first residue is 1, the second residue is 2, etc., an algorithm defining all nucleic acid segments can be created:

n to n+y

where n is an integer from 1 to the last number of the sequence and y is the length of the nucleic acid segment minus one, where n+y does not exceed the last number of the sequence. Thus, for a 10-mer, the nucleic acid segments correspond to bases 1 to 10, 2 to 11, 3 to 12 . . . and/or so on. For a 15-mer, the nucleic acid segments correspond to bases 1 to 15, 2 to 16, 3 to 17 . . . and/or so on. For a 20-mer, the nucleic segments correspond to bases 1 to 20, 2 to 21, 3 to 22 . . . and/or so on.

The nucleic acid(s) of the present invention, regardless of the length of the sequence itself, may be combined with other nucleic acid sequences, including but not limited to, promoters, enhancers, polyadenylation signals, restriction enzyme sites, multiple cloning sites, coding segments, and the like, to create one or more nucleic acid construct(s). The overall length may vary considerably between nucleic acid constructs. Thus, a nucleic acid segment of almost any length may be employed, with the total length preferably being limited by the ease of preparation or use in the intended recombinant nucleic acid protocol.

In a non-limiting example, one or more nucleic acid constructs may be prepared that include a contiguous stretch of nucleotides identical to or complementary to SEQ ID NO:1 or SEQ ID NO:2 or SEQ ID NO:3 or SEQ ID NO:29. A nucleic acid construct may be about 3, about 5, about 8, about 10 to about 14, or about 15, about 20, about 30, about 40, about 50, about 100, about 200, about 500, about 1,000, about 2,000, about 3,000, about 5,000, about 10,000, about 15,000, about 20,000, about 30,000, about 50,000, about 100,000, about 250,000, about 500,000, about 750,000, to about 1,000,000 nucleotides in length, as well as constructs of greater size, up to and including chromosomal sizes (including all intermediate lengths and intermediate ranges), given the advent of nucleic acids constructs such as a yeast artificial chromosome are known to those of ordinary skill in the art. It will be readily understood that “intermediate lengths” and “intermediate ranges”, as used herein, means any length or range including or between the quoted values (i.e. all integers including and between such values). Non-limiting examples of intermediate lengths include about 11, about 12, about 13, about 16, about 17, about 18, about 19, etc.; about 21, about 22, about 23, etc.; about 31, about 32, etc.; about 51, about 52, about 53, etc.; about 101, about 102, about 103, etc.; about 151, about 152, about 153, etc.; about 1,001, about 1002, etc,; about 50,001, about 50,002, etc; about 750,001, about 750,002, etc.; about 1,000,001, about 1,000,002, etc. Non-limiting examples of intermediate ranges include about 3 to about 32, about 150 to about 500,001, about 3,032 to about 7,145, about 5,000 to about 15,000, about 20,007 to about 1,000,003, etc.

The term “a sequence essentially as set forth in SEQ ID NO:1” or “a sequence essentially as set forth in SEQ ID NO:3” or “a sequence essentially as set forth in SEQ ID NO:5” or “a sequence essentially as set forth in SEQ ID NO:29” means that the sequence substantially corresponds to a portion of SEQ ID NO:1, SEQ ID NO:3, SEQ ID NO:5, SEQ ID NO:29 and encodes relatively few amino acids that are not identical to, or a biologically functional equivalent of, the amino acids encoded by SEQ ID NO:1, SEQ ID NO:3, SEQ ID NO:5, and SEQ ID NO:29. Thus, “a sequence essentially as set forth in SEQ ID NO:1” or “a sequence essentially as set forth in SEQ ID NO:3” “a sequence essentially as set forth in SEQ ID NO:5” “a sequence essentially as set forth in SEQ ID NO:29” encompasses nucleic acids, nucleic acid segments, and genes that comprise part or all of the nucleic acid sequences as set forth in SEQ ID NO:1 and/or SEQ ID NO:3 and/or SEQ ID NO:5 and/or SEQ ID NO:29.

The term “biologically functional equivalent” is well understood in the art and is further defined in detail herein. Accordingly, a sequence that has between about 70% and about 80%; or more preferably, between about 81% and about 90%; or even more preferably, between about 91% and about 99%; of amino acids that are identical or functionally equivalent to the amino acids of SEQ ID NO:1 or SEQ ID NO:3 or SEQ ID NO:5 and/or SEQ ID NO:29 will be a sequence that is “essentially as set forth in SEQ ID NO:1” or “a sequence essentially as set forth in SEQ ID NO:3” or “a sequence essentially as set forth in SEQ ID NO:5” or “a sequence essentially as set forth in SEQ ID NO:29”, provided the biological activity of the protein, polypeptide or peptide is maintained.

In certain other embodiments, the invention concerns at least one recombinant vector that include within its sequence a nucleic acid sequence essentially as set forth in SEQ ID NO:1 or SEQ ID NO:3 or SEQ ID NO:5 or SEQ ID NO:29. In particular embodiments, the recombinant vector comprises DNA sequences that encode protein(s), polypeptide(s) or peptide(s) exhibiting tumor suppressor activity.

The term “functionally equivalent codon” is used herein to refer to codons that encode the same amino acid, such as the six codons for arginine and serine, and also refers to codons that encode biologically equivalent amino acids. For optimization of expression of a tumor suppressor gene in human cells, the codons are shown in Table 1 in preference of use from left to right. Thus, the most preferred codon for alanine is thus “GCC”, and the least is “GCG” (see Table 1, below).

TABLE 1

Preferred Human DNA Codons

| | |
|---|---|
| Amino Acids | Codons |

| | | | |
|---|---|---|---|
| Alanine | Ala | A | GCC GCT GCA GCG |
| Cysteine | Cys | C | TGC TGT |
| Aspartic acid | Asp | D | GAC GAT |
| Glutamic acid | Glu | E | GAG GAA |
| Phenylalanine | Phe | F | TTC TTT |
| Glycine | Gly | G | GGC GGG GGA GGT |
| Histidine | His | H | CAC CAT |
| Isoleucine | Ile | I | ATC ATT ATA |
| Lysine | Lys | K | AAG AAA |
| Leucine | Leu | L | CTG CTC TTG CTT CTA TTA |
| Methionine | Met | M | ATG |
| Asparagine | Asn | N | AAC AAT |
| Proline | Pro | P | CCC CCT CCA CCG |
| Glutamine | Gln | Q | CAG CAA |
| Arginine | Arg | R | CGC AGG CGG AGA CGA CGT |
| Serine | Ser | S | AGC TCC TCT AGT TCA TCG |
| Threonine | Thr | I | ACC ACA ACT ACG |
| Valine | Val | V | GTG GTC GTT GTA |
| Tryptophan | Trp | W | TGG |
| Tyrosine | Tyr | Y | TAC TAT |

Information on codon usage in a variety of non-human organisms is known in the art (see for example, Bennetzen and Hall, 1982; Ikemura, 1981a, 1981b, 1982; Grantham et al., 1980, 1981; Wada et al., 1990; each of these references are incorporated herein by reference in their entirety). Thus, it is contemplated that codon usage may be optimized for other animals, as well as other organisms such as fungi, plants, prokaryotes, virus and the like, as well as organelles that contain nucleic acids, such as mitochondria, chloroplasts and the like, based on the preferred codon usage as would be known to those of ordinary skill in the art.

It will also be understood that amino acid sequences or nucleic acid sequences may include additional residues, such as additional N- or C-terminal amino acids or 5′ or 3′ sequences, or various combinations thereof, and yet still be essentially as set forth in one of the sequences disclosed herein, so long as the sequence meets the criteria set forth above, including the maintenance of biological protein, polypeptide or peptide activity where expression of a proteinaceous composition is concerned. The addition of terminal sequences particularly applies to nucleic acid sequences that may, for example, include various non-coding sequences flanking either of the 5′ and/or 3′ portions of the coding region or may include various internal sequences, i.e., introns, which are known to occur within genes.

Excepting intronic and flanking regions, and allowing for the degeneracy of the genetic code, nucleic acid sequences that have between about 70% and about 79%; or more preferably, between about 80% and about 89%; or even more particularly, between about 90% and about 99%; of nucleotides that are identical to the nucleotides of SEQ ID NO:1 or SEQ ID NO:2 or SEQ ID NO:3 or SEQ ID NO:29 will be nucleic acid sequences that are “essentially as set forth in SEQ ID NO:1 or SEQ ID NO:3 or SEQ ID NO:5 or SEQ ID NO:29”.

It will also be understood that this invention is not limited to the particular nucleic acid of SEQ ID NO:1 or SEQ ID NO:3 or SEQ ID NO:5 or SEQ ID NO:29. Recombinant vectors and isolated nucleic acid segments may therefore variously include these coding regions themselves, coding regions bearing selected alterations or modifications in the basic coding region, and they may encode larger polypeptides or peptides that nevertheless include such coding regions or may encode biologically functional equivalent proteins, polypeptide or peptides that have variant amino acids sequences.

The nucleic acids of the present invention encompass biologically functional equivalent tumor suppressor proteins, polypeptides, or peptides. Such sequences may arise as a consequence of codon redundancy or functional equivalency that are known to occur naturally within nucleic acid sequences or the proteins, polypeptides or peptides thus encoded. Alternatively, functionally equivalent proteins, polypeptides or peptides may be created via the application of recombinant DNA technology, in which changes in the protein, polypeptide or peptide structure may be engineered, based on considerations of the properties of the amino acids being exchanged. Changes designed by man may be introduced, for example, through the application of site-directed mutagenesis techniques as discussed herein below, e.g., to introduce improvements or alterations to the antigenicity of the protein, polypeptide or peptide, or to test mutants in order to examine tumor suppressor protein, polypeptide or peptide activity at the molecular level.

Fusion proteins, polypeptides or peptides may be prepared, e.g., where the tumor suppressor coding regions are aligned within the same expression unit with other proteins, polypeptides or peptides having desired functions. Non-limiting examples of such desired functions of expression sequences include purification or immunodetection purposes for the added expression sequences, e.g., proteinaceous compositions that may be purified by affinity chromatography or the enzyme labeling of coding regions, respectively.

Encompassed by the invention are nucleic acid sequences encoding relatively small peptides or fusion peptides, such as, for example, peptides of from about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, about 90, about 91, about 92, about 93, about 94, about 95, about 96, about 97, about 98, about 99, to about 100 amino acids in length, or more preferably, of from about 15 to about 30 amino acids in length; as set forth in SEQ ID NO:2 or SEQ ID NO:4 or SEQ ID NO:6 or SEQ ID NO:30 and also larger polypeptides up to and including proteins corresponding to the full-length sequences set forth in SEQ ID NO:2 or SEQ ID NO:4 or SEQ ID NO:6 or SEQ ID NO:30.

As used herein an “organism” may be a prokaryote, eukaryote, virus and the like. As used herein the term “sequence” encompasses both the terms “nucleic acid” and “proteinaceous” or “proteinaceous composition.” As used herein, the term “proteinaceous composition” encompasses the terms “protein”, “polypeptide” and “peptide.” As used herein “artificial sequence” refers to a sequence of a nucleic acid not derived from sequence naturally occurring at a genetic locus, as well as the sequence of any proteins, polypeptides or peptides encoded by such a nucleic acid. A “synthetic sequence”, refers to a nucleic acid or proteinaceous composition produced by chemical synthesis in vitro, rather than enzymatic production in vitro (i.e. an “enzymatically produced” sequence) or biological production in vivo (i.e. a “biologically produced” sequence).

IV. Proteinaceous Compositions

Embodiments of the invention include compositions comprising at least one proteinaceous molecule, such as protamine or viral-protamine complex or protamine coupled to a linking moiety, such as a ligand or an antibody. As used herein, a “proteinaceous molecule,” “proteinaceous composition,” “proteinaceous compound,” “proteinaceous chain” or “proteinaceous material” generally refers, but is not limited to, a protein of greater than about 200 amino acids or the full length endogenous sequence translated from a gene; a polypeptide of greater than about 100 amino acids; and/or a peptide of from about 3 to about 100 amino acids. All the “proteinaceous” terms described above may be used interchangeably herein.

In certain embodiments the size of the at least one proteinaceous molecule may comprise, but is not limited to, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000, 1100, 1200, 1300, 1400, 1500, 1750, 2000, 2250, 2500 or greater amino molecule residues, and any range derivable therein. The invention includes those lengths of contiguous amino acids of any sequence discussed herein.

As used herein, an “amino molecule” refers to any amino acid, amino acid derivative or amino acid mimic as would be known to one of ordinary skill in the art. In certain embodiments, the residues of the proteinaceous molecule are sequential, without any non-amino molecule interrupting the sequence of amino molecule residues. In other embodiments, the sequence may comprise one or more non-amino molecule moieties. In particular embodiments, the sequence of residues of the proteinaceous molecule may be interrupted by one or more non-amino molecule moieties.

Accordingly, the term “proteinaceous composition” encompasses amino molecule sequences comprising at least one of the 20 common amino acids in naturally synthesized proteins, or at least one modified or unusual amino acid.

In certain embodiments the proteinaceous composition comprises at least one protein, polypeptide or peptide. In further embodiments the proteinaceous composition comprises a biocompatible protein, polypeptide or peptide. As used herein, the term “biocompatible” refers to a substance which produces no significant untoward effects when applied to, or administered to, a given organism according to the methods and amounts described herein. Such untoward or undesirable effects are those such as significant toxicity or adverse immunological reactions. In preferred embodiments, biocompatible protein, polypeptide or peptide containing compositions will generally be mammalian proteins or peptides or synthetic proteins or peptides each essentially free from toxins, pathogens and harmful immunogens.

Proteinaceous compositions may be made by any technique known to those of skill in the art, including the expression of proteins, polypeptides or peptides through standard molecular biological techniques, the isolation of proteinaceous compounds from natural sources, or the chemical synthesis of proteinaceous materials. The nucleotide and protein, polypeptide and peptide sequences for various genes have been previously disclosed, and may be found at computerized databases known to those of ordinary skill in the art. One such database is the National Center for Biotechnology Information's Genbank and GenPept databases. The coding regions for these known genes may be amplified and/or expressed using the techniques disclosed herein or as would be know to those of ordinary skill in the art. Alternatively, various commercial preparations of proteins, polypeptides and peptides are known to those of skill in the art.

In certain embodiments a proteinaceous compound may be purified. Generally, “purified” will refer to a specific or protein, polypeptide, or peptide composition that has been subjected to fractionation to remove various other proteins, polypeptides, or peptides, and which composition substantially retains its activity, as may be assessed, for example, by the protein assays, as would be known to one of ordinary skill in the art for the specific or desired protein, polypeptide or peptide.

In certain embodiments, the proteinaceous composition may comprise at least a part of an antibody, for example, an antibody against a molecule expressed on a cell's surface, to allow a viral protamine complex to be targeted to the cell. As used herein, the term “antibody” is intended to refer broadly to any immunologic binding agent such as IgG, IgM, IgA, IgD and IgE. Generally, IgG and/or IgM are preferred because they are the most common antibodies in the physiological situation and because they are most easily made in a laboratory setting.

The term “antibody” is used to refer to any antibody-like molecule that has an antigen binding region, and includes antibody fragments such as Fab′, Fab, F(ab′)₂, single domain antibodies (DABs), Fv, scFv (single chain Fv), and the like. The techniques for preparing and using various antibody-based constructs and fragments are well known in the art. Means for preparing and characterizing antibodies are also well known in the art (See, e.g., Harlow et al., 1988; incorporated herein by reference).

It is contemplated that virtually any protein, polypeptide or peptide containing component may be used in the compositions and methods disclosed herein. However, it is preferred that the proteinaceous material is biocompatible. In certain embodiments, it is envisioned that the formation of a more viscous composition will be advantageous in that will allow the composition to be more precisely or easily applied to the tissue and to be maintained in contact with the tissue throughout the procedure. In such cases, the use of a peptide composition, or more preferably, a polypeptide or protein composition, is contemplated. Ranges of viscosity include, but are not limited to, about 40 to about 100 poise. In certain aspects, a viscosity of about 80 to about 100 poise is preferred.

A. Functional Aspects

When the present application refers to the function or activity of protamine, it is meant that the molecule in question helps to precipitate a nucleic acid molecule. Determination of which molecules possess this activity may be achieved using assays familiar to those of skill in the art.

On the other hand, when the present invention refers to the function or activity of a “targeting moiety” one of ordinary skill in the art would further understand that this includes, for example, the ability to specifically bind a particular compound or molecule, thus allowing for targeting of the compound or molecule or a cell having the compound or molecule. Determination of which molecules are suitable targeting moieties may be achieved using assays familiar to those of skill in the art—some of which are disclosed herein—and may include, for example, the use of native and/or recombinant tumor suppressors.

B. Variants of Proteinaceous Compositions

Amino acid sequence variants of the polypeptides and peptides of the present invention can be substitutional, insertional or deletion variants. Deletion variants lack one or more residues of the native protein that are not essential for function or immunogenic activity, and are exemplified by the variants lacking a transmembrane sequence described above. Another common type of deletion variant is one lacking secretory signal sequences or signal sequences directing a protein to bind to a particular part of a cell. Insertional mutants typically involve the addition of material at a non-terminal point in the polypeptide. This may include the insertion of an immunoreactive epitope or simply a single residue. Terminal additions, called fusion proteins, are discussed below.

Substitutional variants typically contain the exchange of one amino acid for another at one or more sites within the protein, and may be designed to modulate one or more properties of the polypeptide, such as stability against proteolytic cleavage, without the loss of other functions or properties. Substitutions of this kind preferably are conservative, that is, one amino acid is replaced with one of similar shape and charge. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine.

The term “biologically functional equivalent” is well understood in the art and is further defined in detail herein. Accordingly, sequences that have between about 70% and about 80%; or more preferably, between about 81% and about 90%; or even more preferably, between about 91% and about 99%; of amino acids that are identical or functionally equivalent to the amino acids of the protamine or a linking moiety provided the biological activity of the protein is maintained. (see Table 2, below for a list of functionally equivalent codons).

The following is a discussion based upon changing of the amino acids of a protein to create an equivalent, or even an improved, second-generation molecule. For example, certain amino acids may be substituted for other amino acids in a protein structure without appreciable loss of interactive binding capacity with structures such as, for example, antigen-binding regions of antibodies or binding sites on substrate molecules. Since it is the interactive capacity and nature of a protein that defines that protein's biological functional activity, certain amino acid substitutions can be made in a protein sequence, and in its underlying DNA coding sequence, and nevertheless produce a protein with like properties. It is thus contemplated by the inventors that various changes may be made in the DNA sequences of genes without appreciable loss of their biological utility or activity, as discussed below.

TABLE 2

Codon Table

| | |
|---|---|
| Amino Acids | Codons |

| | | | |
|---|---|---|---|
| Alanine | Ala | A | GCA GCC GCG GCU |
| Cysteine | Cys | C | UGC UGU |
| Aspartic acid | Asp | D | GAC GAU |
| Glutamic acid | Glu | E | GAA GAG |
| Phenylalanine | Phe | F | UUC UUU |
| Glycine | Gly | G | GGA GGC GGG GGU |
| Histidine | His | H | CAC CAU |
| Isoleucine | Ile | I | AUA AUC AUU |
| Lysine | Lys | K | AAA AAG |
| Leucine | Leu | L | UUA UUG CUA CUC CUG CUU |
| Methionine | Met | M | AUG |
| Asparagine | Asn | N | AAC AAU |
| Proline | Pro | P | CCA CCC CCG CCU |
| Glutamine | Gln | Q | CAA CAG |
| Arginine | Arg | R | AGA AGG CGA CGC CGG CGU |
| Serine | Ser | S | AGC AGU UCA UCC UCG UCU |
| Threonine | Thr | T | ACA ACC ACG ACU |
| Valine | Val | V | GUA GUC GUG GUU |
| Tryptophan | Trp | W | UGG |
| Tyrosine | Tyr | Y | UAC UAU |

In making such changes, the hydropathic index of amino acids may be considered. The importance of the hydropathic amino acid index in conferring interactive biologic function on a protein is generally understood in the art (Kyte & Doolittle, 1982). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like.

It also is understood in the art that the substitution of like amino acids can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5 ±1); alanine (−0.5); histidine *−0.5); cysteine (−1.0); methionine (−1.3); valine (−1.5); leucine (−1.8); isoleucine (−1.8); tyrosine (−2.3); phenylalanine (−2.5); tryptophan (−3.4).

It is understood that an amino acid can be substituted for another having a similar hydrophilicity value and still produce a biologically equivalent and immunologically equivalent protein. In such changes, the substitution of amino acids whose hydrophilicity values are within ±2 is preferred, those that are within ±1 are particularly preferred, and those within ±0.5 are even more particularly preferred.

As outlined above, amino acid substitutions generally are based on the relative similarity of the amino acid side-chain substituents, for example, their hydrophobicity, hydrophilicity, charge, size, and the like. Exemplary substitutions that take into consideration the various foregoing characteristics are well known to those of skill in the art and include: arginine and lysine; glutamate and aspartate; serine and threonine; glutamine and asparagine; and valine, leucine and isoleucine.

Another embodiment for the preparation of polypeptides according to the invention is the use of peptide mimetics. Mimetics are peptide-containing molecules that mimic elements of protein secondary structure. See e.g., Johnson (1993). The underlying rationale behind the use of peptide mimetics is that the peptide backbone of proteins exists chiefly to orient amino acid side chains in such a way as to facilitate molecular interactions, such as those of antibody and antigen. A peptide mimetic is expected to permit molecular interactions similar to the natural molecule. These principles may be used, in conjunction with the principles outline above, to engineer second generation molecules having many of the natural properties of protamine or a linking moiety, but with altered and even improved characteristics.

C. Fusion Proteins

A specialized kind of insertional variant is the fusion protein. This molecule generally has all or a substantial portion of the native molecule, linked at the N- or C-terminus, to all or a portion of a second polypeptide. In the present invention, a fusion may comprise a protamine sequence and a linking moiety. In other examples, fusions employ leader sequences from other species to permit the recombinant expression of a protein in a heterologous host. Another useful fusion includes the addition of an immunologically active domain, such as an antibody epitope, to facilitate purification of the fusion protein. Inclusion of a cleavage site at or near the fusion junction will facilitate removal of the extraneous polypeptide after purification. Other useful fusions include linking of functional domains, such as active sites from enzymes such as a hydrolase, glycosylation domains, cellular targeting signals or transmembrane regions.

Following transduction with an expression construct or vector according to some embodiments of the present invention, primary mammalian cell cultures may be prepared in various ways. In order for the cells to be kept viable while in vitro and in contact with the expression construct, it is necessary to ensure that the cells maintain contact with the correct ratio of oxygen and carbon dioxide and nutrients but are protected from microbial contamination. Cell culture techniques are well documented and are disclosed herein by reference (Freshner, 1992).

One embodiment of the foregoing involves the use of gene transfer to immortalize cells for the production and/or presentation of proteins. The gene for the protein of interest may be transferred as described above into appropriate host cells followed by culture of cells under the appropriate conditions. The gene for virtually any polypeptide may be employed in this manner. The generation of recombinant expression vectors, and the elements included therein, are discussed above. Alternatively, the protein to be produced may be an endogenous protein normally synthesized by the cell in question.

Another embodiment of the present invention uses cell lines, which are transfected with an expression construct or vector that expresses a therapeutic protein such as a tumor suppressor. Examples of mammalian host cell lines include Vero and HeLa cells, other B- and T-cell lines, such as CEM, 721.221, H9, Jurkat, Raji, etc., as well as cell lines of Chinese hamster ovary, W138, BHK, COS-7, 293, HepG2, 3T3, RIN and MDCK cells. In addition, a host cell strain may be chosen that modulates the expression of the inserted sequences, or that modifies and processes the gene product in the manner desired. Such modifications (e.g., glycosylation) and processing (e.g., cleavage) of protein products may be important for the function of the protein. Different host cells have characteristic and specific mechanisms for the post-translational processing and modification of proteins. Appropriate cell lines or host systems can be chosen to insure the correct modification and processing of the foreign protein expressed.

A number of selection systems may be used including, but not limited to, HSV thymidine kinase, hypoxanthine-guanine phosphoribosyltransferase and adenine phosphoribosyltransferase genes, in tk-, hgprt- or aprt-cells, respectively. Also, anti-metabolite resistance can be used as the basis of selection: for dhfr, which confers resistance to; gpt, which confers resistance to mycophenolic acid; neo, which confers resistance to the aminoglycoside G418; and hygro, which confers resistance to hygromycin.

Animal cells can be propagated in vitro in two modes: as non-anchorage-dependent cells growing in suspension throughout the bulk of the culture or as anchorage-dependent cells requiring attachment to a solid substrate for their propagation (i.e., a monolayer type of cell growth).

Non-anchorage dependent or suspension cultures from continuous established cell lines are the most widely used means of large-scale production of cells and cell products. However, suspension cultured cells have limitations, such as tumorigenic potential and lower protein production than adherent cells.

V. Therapeutic Formulations and Routes of Administration

Embodiments of the invention include compositions and methods involving a viral composition for improved transduction efficiency, therapeutic efficacy and a decreased viral vector-reduced toxicity for delivering selective agents to a cancer cell. While systemic administration of formulations can provide a treatment method, frequently this delivery method fails to reach a location where it can confer a therapeutic benefit or it does so with reduced efficacy. The invention includes methods and compositions for systemic administration. Certain embodiments, include a targeting method that allows the delivery of viral compositions to mucosal epithelia and other cell types.

Where clinical applications are contemplated, it will be necessary to prepare the compositions of the present invention as pharmaceutically acceptable compositions, i.e., in a form appropriate for in vivo applications. Generally, this will entail preparing compositions that are essentially free of pyrogens, as well as other impurities that could be harmful to humans or animals.

A. Preparation Methods

The compounds of the invention include a viral composition comprising a viral vector and a protamine molecule. In some embodiments, a composition may include a therapeutic agent or a diagnostic agent. The protamine molecule or viral vectors of the invention may be linked, or operatively attached, to the therapeutic or diagnostic agent by either chemical conjugation (e.g., crosslinking) or through recombinant DNA techniques.

The present invention provides a method of preparing a viral composition comprising preparing a first solution comprising a viral vector, having a polynucleotide encoding a tumor suppressor, in a concentration of about 10¹⁰ viral particles per 50 μL diluent; preparing a second solution comprising a protamine molecule in a concentration of about 100 to 300 μg per 50 μL diluent; mixing the first solution with the second solution in a ratio of about 1:1, 1:2, 1:4, 2:1, 4:1 and so on to form a third solution; and incubating the third solution for a time sufficient to effect coordination between the viral vector and the protamine molecule and produce the viral composition.

Embodiments of the invention include methods that further comprises the step of adding the viral composition to a pharmacologically acceptable diluent at a therapeutically effective concentration. In one specific embodiment, the concentration is in a range between about 1×10¹⁰ to about 5×10¹¹ viral particles. The viral vector may be an adenoviral vector, a retroviral vector, a vaccinia viral vector, an adeno-associated viral vector, a polyoma viral vector, or a herpes viral vector.

Embodiments of the invention include a viral composition prepared by the process comprising preparing a first solution comprising a viral vector having a polynucleotide encoding a tumor suppressor in a concentration of about 10¹⁰, about 10¹¹, about 10¹², about 10¹³, about 10¹⁴, or about 10¹⁵ viral particles per 20, 25, 30, 40, 45, 50, 55, 60, 65, 70, 75, 80 μL or more diluent; preparing a second solution comprising a protamine molecule in a concentration of about 100, 125, 150, 175, 200, 225, 250, 275, or 300 μg per 20, 25, 30, 40, 45, 50, 55, 60, 65, 70, 75, 80 μL or more diluent; mixing the first solution with the second solution in a ratio of about 4:1, 2:1, 1:1, 1:2, 1:4 and so on to form a third solution; and incubating the third solution for a time sufficient to effect complex formation between the viral vector and the protamine molecule to produce a viral composition.

B. Formulations and Administrations

One will generally desire to employ appropriate salts and buffers to render delivery vectors and compositions stable and allow for uptake by target cells. Buffers also will be employed when recombinant cells are introduced into a patient. Aqueous compositions of the present invention comprise an effective amount of the viral composition to cells, dissolved or dispersed in a pharmaceutically acceptable carrier or aqueous medium. Such compositions also are referred to as inocula. The phrase “pharmaceutically or pharmacologically acceptable” refer to compositions and/or molecular entities that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well know in the art. Except insofar as any conventional media or agent is incompatible with the vectors or cells of the present invention, its use in therapeutic compositions is contemplated. Supplementary active ingredients also can be incorporated into the compositions.

The active compositions of the present invention include classic pharmaceutical preparations. Administration of these compositions according to the present invention will be via any common route so long as the target tissue is available via that route. This includes oral, nasal, buccal, rectal, vaginal or topical. Alternatively, administration may be by orthotopic, intradermal, subcutaneous, intralesional, intramuscular, intraperitoneal or intravenous. Such compositions would normally be administered as pharmaceutically acceptable compositions, described supra.

The active compounds may be administered via any suitable route, including parenterally, intravascularly or by direct injection or inhalation. Solutions of the active compounds as free base or pharmacologically acceptable salts can be prepared in water suitably mixed with a surfactant, such as hydroxypropylcellulose. Dispersions also can be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial an antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients also can be incorporated into the compositions.

The compositions of the present invention may be formulated in a neutral or salt form. Pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups also can be derived from inorganic bases such as, for example, sodium, potassium, ammonium, calcium, or ferric hydroxides, and such organic bases as isopropylamine, trimethylamine, histidine, procaine and the like.

Upon formulation, solutions will be administered in a manner compatible with the dosage formulation and in such amount as is therapeutically effective. The formulations are easily administered in a variety of dosage forms such as injectable solutions, drug release capsules and the like. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration.

The present invention can be administered intravascularly, intravenously, intradermally, intraarterially, intraperitoneally, intralesionally, intracranially, intraarticularly, intraprostaticaly, intrapleurally, intratracheally, intranasally, intravitreally, intravaginally, intrarectally, topically, intratumorally, intramuscularly, intraperitoneally, subcutaneously, subconjunctival, intravesicularlly, mucosally, intrapericardially, intraumbilically, intraocularally, orally, topically, locally, inhalation (e.g., aerosol inhalation), injection, infusion, continuous infusion, localized perfusion bathing target cells directly, via a catheter, via a lavage, in cremes, in lipid compositions (e.g., liposomes), or by other method or any combination of the forgoing as would be known to one of ordinary skill in the art (see, for example, Remington's Pharmaceutical Sciences, 18th Ed. Mack Printing Company, 1990, incorporated herein by reference). Additional formulations which are suitable for other modes of administration include suppositories and, in some cases, oral formulations. For suppositories, traditional binders and carriers may include, for example, polyalkalene glycols or triglycerides: such suppositories may be formed from mixtures containing the active ingredient in the range of about 0.5% to about 10%, preferably about 1 to about 2%. Oral formulations include such normally employed excipients as, for example, pharmaceutical grades of mannitol, lactose, starch, magnesium stearate, sodium saccharine, cellulose, magnesium carbonate and the like. These compositions take the form of solutions, suspensions, tablets, pills, capsules, sustained release formulations or powders and contain about 10 to about 95% of active ingredient, preferably about 25 to about 70%.

One may also use nasal solutions or sprays, aerosols or inhalants in the present invention. Nasal solutions are usually aqueous solutions designed to be administered to the nasal passages in drops or sprays. Nasal solutions are prepared so that they are similar in many respects to nasal secretions, so that normal ciliary action is maintained. Thus, the aqueous nasal solutions usually are isotonic and slightly buffered to maintain a pH of 5.5 to 6.5.

In addition, antimicrobial preservatives, similar to those used in ophthalmic preparations, and appropriate drug stabilizers, if required, may be included in the formulation. Various commercial nasal preparations are known and include, for example, antibiotics and antihistamines and are used for asthma prophylaxis.

In certain embodiments, active compounds may be administered orally. This is contemplated to be useful as many substances contained in tablets designed for oral use are absorbed by mucosal epithelia along the gastrointestinal tract.

Also, if desired, the peptides, antibodies and other agents may be rendered resistant, or partially resistant, to proteolysis by digestive enzymes. Such compounds are contemplated to include chemically designed or modified agents; dextrorotatory peptides; and peptide and liposomal formulations in time release capsules to avoid peptidase and lipase degradation.

For oral administration, the active compounds may be administered, for example, with an inert diluent or with an assimilable edible carrier, or they may be enclosed in hard or soft shell gelatin capsule, or compressed into tablets, or incorporated directly with the food of the diet. For oral therapeutic administration, the active compounds may be incorporated with excipients and used in the form of ingestible tablets, buccal tables, troches, capsules, elixirs, suspensions, syrups, wafers, and the like.

Various other materials may be present as coatings or to otherwise modify the physical form of the dosage unit. For instance, tablets, pills, or capsules may be coated with shellac, sugar or both. A syrup of elixir may contain the active compounds sucrose as a sweetening agent methyl and propylparabens as preservatives, a dye and flavoring, such as cherry or orange flavor. Of course, any material used in preparing any dosage unit form should be pharmaceutically pure and substantially non-toxic in the amounts employed. In addition, the active compounds may be incorporated into sustained-release preparation and formulations.

Upon formulation, the compounds will be administered in a manner compatible with the dosage formulation and in such amount as is therapeutically effective. The formulations are easily administered in a variety of dosage forms, as described herein.

C. Vaccines

The present invention contemplates vaccines for use in both active and passive immunization embodiments. Immunogenic compositions, proposed to be suitable for use as a vaccine, may be prepared most readily directly from immunogenic calcium binding peptides prepared in a manner disclosed herein. Preferably the antigenic material is extensively dialyzed to remove undesired small molecular weight molecules and/or lyophilized for more ready formulation into a desired vehicle.

The preparation of vaccines that comprise a viral vector and a protamine molecule are contemplated. (See U.S. Pat. Nos. 4,608,251; 4,601,903; 4,599,231; and 4,599,230, all incorporated herein by reference.) Typically, vaccines are prepared as injectables. Either as liquid solutions or suspensions: solid forms suitable for solution in, or suspension in, liquid prior to injection may also be prepared. The preparation may also be emulsified. The active immunogenic ingredient is often mixed with excipients which are pharmaceutically acceptable and compatible with the active ingredient. Suitable excipients are, for example, water, saline, dextrose, glycerol, ethanol, or the like and combinations thereof. In addition, if desired, the vaccine may contain minor amounts of auxiliary substances such as wetting or emulsifying agents, pH buffering agents, or adjuvants which enhance the effectiveness of the vaccines. Additionally, iscom, a supramolecular spherical structure, may be used for parenteral and mucosal vaccination (Morein et al., 1998).

Vaccines may be conventionally administered parenterally, by injection, for example, either subcutaneously or intramuscularly. Additional formulations which are suitable for other modes of administration include suppositories and, in some cases, oral formulations. For suppositories, traditional binders and carriers may include, for example, polyalkalene glycols or triglycerides: such suppositories may be formed from mixtures containing the active ingredient in the range of about 0.5% to about 10%, preferably about 1 to about 2%. Oral formulations include such normally employed excipients as, for example, pharmaceutical grades of mannitol, lactose, starch, magnesium stearate, sodium saccharine, cellulose, magnesium carbonate and the like. These compositions take the form of solutions, suspensions, tablets, pills, capsules, sustained release formulations or powders and contain about 10 to about 95% of active ingredient, preferably about 25 to about 70%.

The protamine-Ad complexes of the present invention may be formulated into the vaccine as neutral or salt forms. Pharmaceutically-acceptable salts, include the acid addition salts (formed with the free amino groups of the peptide) and those which are formed with inorganic acids such as, for example, hydrochloric or phosphoric acids, or such organic acids as acetic, oxalic, tartaric, mandelic, and the like. Salts formed with the free carboxyl groups may also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

The vaccines are administered in a manner compatible with the dosage formulation, and in such amount as will be therapeutically effective and immunogenic. The quantity to be administered depends on the subject to be treated, including, e.g., the capacity of the individual's immune system to synthesize antibodies, and the degree of protection desired. Precise amounts of active ingredient required to be administered depend on the judgment of the practitioner. However, suitable dosage ranges are of the order of several hundred micrograms active ingredient per vaccination. Suitable regimes for initial administration and booster shots are also variable, but are typified by an initial administration followed by subsequent inoculations or other administrations.

The manner of application may be varied widely. Any of the conventional methods for administration of a vaccine are applicable. These are believed to include oral application on a solid physiologically acceptable base or in a physiologically acceptable dispersion, parenterally, by injection or the like. The dosage of the vaccine will depend on the route of administration and will vary according to the size of the host.

Various methods of achieving adjuvant effect for the vaccine includes use of agents such as aluminum hydroxide or phosphate (alum), commonly used as about 0.05 to about 0.1% solution in phosphate buffered saline, admixture with synthetic polymers of sugars (Carbopol®) used as an about 0.25% solution, aggregation of the protein in the vaccine by heat treatment with temperatures ranging between about 70° to about 101° C. for a 30-second to 2-minute period, respectively. Aggregation by reactivating with pepsin treated (Fab) antibodies to albumin, mixture with bacterial cells such as C. parvum or endotoxins or lipopolysaccharide components of Gram-negative bacteria, emulsion in physiologically acceptable oil vehicles such as mannide mono-oleate (Aracel A) or emulsion with a 20% solution of a perfluorocarbon (Fluosol-DA®) used as a block substitute may also be employed.

In many instances, it will be desirable to have multiple administrations of the vaccine, usually not exceeding six vaccinations, more usually not exceeding four vaccinations and preferably one or more, usually at least about three vaccinations. The vaccinations will normally be at from two to twelve week intervals, more usually from three to five week intervals. Periodic boosters at intervals of 1-5 years, usually three years, will be desirable to maintain protective levels of the antibodies. The course of the immunization may be followed by assays for antibodies for the supernatant antigens. The assays may be performed by labeling with conventional labels, such as radionuclides, enzymes, fluorescents, and the like. These techniques are well known and may be found in a wide variety of patents, such as U.S. Pat. Nos. 3,791,932; 4,174,384 and 3,949,064, as illustrative of these types of assays. “Unit dose” is defined as a discrete amount of a therapeutic composition dispersed in a suitable carrier. For example, in accordance with the present methods, viral doses include a particular number of virus particles or plaque forming units (pfu). For embodiments involving adenovirus, particular unit doses include 10³, 10⁴, 10⁵, 10⁶, 10⁷, 10⁸, 10⁹, 10¹⁰, 10¹¹, 10¹², 10¹³, 10¹⁴ or 10¹⁵ pfu or viral particles. Particle doses may be somewhat higher (10 to 100-fold) due to the presence of infection defective particles.

In this connection, sterile aqueous media which can be employed will be known to those of skill in the art in light of the present disclosure. For example, a unit dose could be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

In some embodiments, the present invention is directed at the treatment of human malignancies. A variety of different routes of administration are contemplated. For example, a classic and typical therapy will involve direct, intratumoral injection of a discrete tumor mass. The injections may be single or multiple; where multiple, injections are made at about 1 cm spacings across the accessible surface of the tumor. Alternatively, targeting the tumor vasculature by direct, local or regional intra-arterial injection are contemplated. The lymphatic systems, including regional lymph nodes, present another likely target given the potential for metastasis along this route. Further, systemic injection may be preferred when specifically targeting secondary (i.e., metastatic) tumors.

In another embodiment, the viral gene therapy may precede or following resection of the tumor. Where prior, the gene therapy may, in fact, permit tumor resection where not possible before. Alternatively, a particularly advantageous embodiment involves the prior resection of a tumor (with or without prior viral gene therapy), followed by treatment of the resected tumor bed. This subsequent treatment is effective at eliminating microscopic residual disease which, if left untreated, could result in regrowth of the tumor. This may be accomplished, quite simply, by bathing the tumor bed with a viral preparation containing a unit dose of viral vector. Another preferred method for achieving the subsequent treatment is via catheterization of the resected tumor bed, thereby permitting continuous perfusion of the bed with virus over extended postoperative periods.

VI. Combined Therapy with Protamine-Ad Complex

In many therapies, it will be advantageous to provide more than one functional therapeutic. Such “combined” therapies may have particular importance in treating aspects of multidrug resistant (MDR) cancers and in antibiotic resistant bacterial infections. Thus, one aspect of the present invention utilizes a viral composition comprising a viral vector encoding a tumor suppressor and a protamine molecule to deliver therapeutic compounds or polynucleotides for treatment of diseases, while a second therapy, either targeted or non-targeted, also is provided.

The non-targeted treatment may precede or follow the targeted agent treatment by intervals ranging from minutes to weeks. In embodiments where the other agent and expression construct are applied separately to the cell, one would generally ensure that a significant period of time did not expire between the time of each delivery, such that the agent and expression construct would still be able to exert an advantageously combined effect on the cell. In such instances, it is contemplated that one would contact the cell with both modalities within about 12-24 hours of each other and, more preferably, within about 6-12 hours of each other, with a delay time of only about 12 hours being most preferred. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

It also is conceivable that more than one administration of either agent will be desired. Various combinations may be employed, where an inventive viral composition is “A” and the non-targeted agent is “B”, as exemplified below:

| | | | | | | | |
|---|---|---|---|---|---|---|---|
| A/B/A | B/A/B | B/B/A | A/A/B | B/A/A | A/B/B | B/B/B/A | B/B/A/B |

| | | | | | | |
|---|---|---|---|---|---|---|
| A/A/B/B | A/B/A/B | A/B/B/A | B/B/A/A | B/A/B/A | B/A/A/B | B/B/B/A |
| A/A/A/B | B/A/A/A | A/B/A/A | A/A/B/A | A/B/B/B | B/A/B/B | B/B/A/B |

Other combinations are contemplated. For example, in the context of the present invention, it is contemplated that gene therapy of the present invention could be used in conjunction with non-targeted anti-cancer agents, including chemo- or radiotherapeutic intervention. To kill cells, inhibit cell growth, inhibit metastasis, inhibit angiogenesis or otherwise reverse or reduce the malignant phenotype of tumor cells, using the methods and compositions of the present invention, one would generally contact a “target” cell with a targeting agent/therapeutic agent and at least one other agent; these compositions would be provided in a combined amount effective achieve these goals. This process may involve contacting the cells with the expression construct and the agent(s) or factor(s) at the same time. This may be achieved by contacting the cell with a single composition or pharmacological formulation that includes both agents, or by contacting the cell with two distinct compositions or formulations, at the same time, wherein one composition includes the expression construct and the other includes the agent. Alternatively, a gene therapy treatment involving a tumor suppressor gene, an antisense oncogene or oncogene-specific ribozyme may be used.

Agents or factors suitable for use in a combined cancer therapy are any chemical compound or treatment method with anticancer activity; therefore, the term “anticancer agent” that is used throughout this application refers to an agent with anticancer activity. These compounds or methods include alkylating agents, topisomerase I inhibitors, topoisomerase II inhibitors, RNA/DNA antimetabolites, DNA antimetabolites, antimitotic agents, as well as DNA damaging agents, which induce DNA damage when applied to a cell.

Examples of alkylating agents include, inter alia, chloroambucil, cis-platinum, cyclodisone, flurodopan, methyl CCNU, piperazinedione, teroxirone. Topisomerase I inhibitors encompass compounds such as camptothecin and camptothecin derivatives, as well as morpholinodoxorubicin. Doxorubicin, pyrazoloacridine, mitoxantrone, and rubidazone are illustrations of topoisomerase II inhibitors. RNA/DNA antimetabolites include L-alanosine, 5-fluoraouracil, aminopterin derivatives, methotrexate, and pyrazofurin; while the DNA antimetabolite group encompasses, for example, ara-C, guanozole, hydroxyurea, thiopurine. Typical antimitotic agents are colchicine, rhizoxin, taxol, and vinblastine sulfate. Other agents and factors include radiation and waves that induce DNA damage such as, γ-irradiation, X-rays, UV-irradiation, microwaves, electronic emissions, and the like. A variety of anti-cancer agents, also described as “chemotherapeutic agents,” function to induce DNA damage, all of which are intended to be of use in the combined treatment methods disclosed herein. Chemotherapeutic agents contemplated to be of use, include, e.g., adriamycin, bleomycin, 5-fluorouracil (5FU), etoposide (VP-16), camptothecin, actinomycin-D, mitomycin C, cisplatin (CDDP), podophyllotoxin, verapamil, and even hydrogen peroxide. The invention also encompasses the use of a combination of one or more DNA damaging agents, whether radiation-based or actual compounds, such as the use of X-rays with cisplatin or the use of cisplatin with etoposide.

The skilled artisan is directed to “Remington's Pharmaceutical Sciences” 15th Edition, chapter 33, in particular pages 624-652. Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

The inventors propose that local, regional delivery of a therapeutic/preventative agent targeted to a malignancy in patients with cancers, precancers, or hyperproliferative conditions will be a very efficient method for delivering a therapeutically effective compound to counteract the clinical disease. Similarly, the chemo- or radiotherapy may be directed to a particular, affected region of the subjects body. Alternatively, systemic delivery of compounds and/or the agents may be appropriate in certain circumstances, for example, where extensive metastasis has occurred.

In addition to combination therapies with chemo- and radiotherapies, it also is contemplated that combination with other gene therapies will be advantageous. For example, targeting of a malignancy using a combination of p53, p16, p21, Rb, APC, DCC, NF-1, NF-2, BCRA2, p16, FHIT, WT-1, MEN-I, MEN-II, BRCA1, VHL, FCC, or MCC, or antisense versions of the oncogenes ras, myc, neu, raf, erb, src, fms, jun, trk, ret, gsp, hst, bcl or abl are included within the scope of the invention.

VII. Assays

Embodiments of the invention include compositions that provide increased transduction efficiency. Such compositions may be tested in vitro, for transduction efficiency, and in vivo, for therapeutic efficacy, viral-induced toxicity, and the like. The various assays for use in determining such changes in function are routine to those of ordinary skill in the art.

In vitro assays involve the use of an isolated viral composition or cells transfected with the viral composition. A convenient way to monitor transduction efficiency is by use of a detectable label, and assess the quantity of the label in the cellular population. Alternatively, a functional read out may be preferred, for example, the ability to affect (kill, promote or inhibit the growth of) a target cell or a host cell.

Some vectors may employ control sequences that allow it to be replicated and/or expressed in both prokaryotic and eukaryotic cells. One of skill in the art would further understand the conditions under which to incubate all of the above described host cells to maintain them and to permit replication of a vector. Also understood and known are techniques and conditions that would allow large-scale production of vectors, as well as production of the nucleic acids encoded by vectors and their cognate polypeptides, proteins, or peptides.

In vivo assays, such as an MDCK transcytosis system assay, also can be easily conducted (Mostov et al., 1986). In these systems, it again is generally preferred to label the test candidate constructs with a detectable marker and to follow the presence of the marker after administration to the animal, preferably via the route intended in the ultimate therapeutic treatment strategy. As part of this process, one would take samples of body fluids, and one would analyze the samples for the presence of the marker associated with the viral composition.

Other compounds are known in the art to serve as diagnostic compounds. For example, protein conjugates in which a protein sequence such as a peptide having a therapeutic activity or a viral composition or a protamine molecule is linked to a detectable label. “Detectable labels” are compounds or elements that can be detected due to their specific functional properties, or chemical characteristics, the use of which allows the peptide or protein to which they are attached to be detected, and further quantified if desired.

Many appropriate imaging agents are known in the art, as are methods for their attachment to proteins (see, e.g., U.S. Pat. Nos. 5,021,236 and 4,472,509, both incorporated herein by reference). Certain attachment methods involve the use of a metal chelate complex employing, for example, an organic chelating agent such a DTPA attached to the antibody (U.S. Pat. No. 4,472,509). Protein sequences may also be reacted with an enzyme in the presence of a coupling agent such as glutaraldehyde or periodate. Conjugates with fluorescein markers are prepared in the presence of these coupling agents or by reaction with an isothiocyanate. Rhodamine markers can also be prepared.

In the case of paramagnetic ions, one might mention by way of example ions such as chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III) and erbium (III), with gadolinium being particularly preferred.

Ions useful in other contexts, such as X-ray imaging, include but are not limited to lanthanum (III), gold (III), lead (II), and especially bismuth (III). In the case of radioactive isotopes for therapeutic and/or diagnostic application, one might mention astatine²¹¹, carbon¹⁴, chromium⁵¹, chlorine³⁶, cobalt⁵⁷, cobalt58, copper⁶⁷, Eu¹⁵², gallium⁶⁷, hydrogen³, iodine¹²³, iodine¹²⁵, iodine¹³¹, indium¹¹¹, iron⁵⁹, phosphorus³², rhenium¹⁸⁶, rhenium¹⁸⁸, selenium⁷⁵, sulphur³⁵, technicium^{99m} and yttrium⁹⁰. Iodine¹²⁵ is often being preferred for use in certain embodiments, and technicium^{99m} and indium¹¹¹ are also often preferred due to their low energy and suitability for long range detection.

VIII. Protamine Conjugates

Embodiments of the invention include viral compositions comprising a viral vector having a polynucleotide encoding a first therapeutic molecule, and a protamine molecule conjugated to a targeting moiety. In preferred embodiments, the targeting moiety is a site-directing or targeting compound that improves the compositions ability to be localized or site-specific in the host. The therapeutic compound may be a nucleic acid molecule, small molecule, or it may be a proteinaceous compound, as discussed herein.

A. Therapeutic Compounds

A targeting moiety of the present invention may be operatively linked or attached to the protamine. Different and varied therapeutic compounds are illustrated. These include enzymes, drugs (e.g., antibacterial, antifungal, anti-viral), antibody regions, regions that mediate protein-protein or ligand receptor interactions, cytokines, growth factors, hormones, toxins, polynucleotides coding for proteins, antisense sequences, radiotherapeutics, chemotherapeutics, ribozymes, tumor suppressors, transcription factors, inducers of apoptosis, or liposomes containing any of the foregoing. In addition to encompassing the delivery of purified compounds, the present invention further contemplates the delivery of nucleic acids that encode cognate compounds such as polypeptides. Therefore, according to the present invention, both purified compounds and nucleic acid sequences encoding that compound, e.g., a cytokine, may be delivered in conjunction with the composition of the present invention.

1. Tumor Suppressors

A number of proteins have been characterized as tumor suppressors, which define a class of proteins that are involved with regulated cell proliferation. The loss of wild-type tumor suppressor activity is associated with neoplastic or unregulated cell growth. It has been shown by several groups that the neoplastic growth of cells lacking a wild-type copy of a particular tumor suppressor can be halted by the addition of a wild-type version of that tumor suppressor (Diller et al., 1990). The present invention contemplates the use of a protamine molecule for the delivery of a tumor suppressor, such as p53. Other tumor suppressors that may be employed according to the present invention include p21, p15, BRCA1, BRCA2, IRF-1, PTEN (MMAC1), Rb, APC, DCC, NF-1, NF-2, WT-1, MEN-I, MEN-II, zacI, p73, VHL, FCC, and MCC.

2. Enzymes

Various enzymes are of interest according to the present invention. Enzymes that could be conjugated to the protamine molecule, either directly or through a linking moiety, include cytosine deaminase, adenosine deaminase, hypoxanthine-guanine phosphoribosyltransferase, galactose-1-phosphate uridyltransferase, phenylalanine hydroxylase, glucose-6-phosphate dehydrogenase, HSV thymidine kinase, and human thymidine kinase and extracellular proteins such as collagenase and matrix metalloprotease, lysosomal glucosidase (Pompe's disease), muscle phosphorylase (McArdle's syndrome), glucocerebosidase (Gaucher's disease), α-L-iduronidase (Hurler syndrome), L-iduronate sulfatase (Hunter syndrome), sphingomyelinase (Niemann-Pick disease) and hexosaminidase (Tay-Sachs disease).

3. Drugs

According to the present invention, a drug may be operatively linked to a vector, or a linking moiety to deliver the drug to the mucosal epithelia. It is contemplated that drugs such as antimetabolites (e.g., purine analogs, pyrimidine analogs, folic acid analogs), enzyme inhibitors, metabolites, or antibiotics (e.g., mitomycin) are useful in the present invention. Small molecules are also included.

4. Antibody Regions

Regions from the various members of the immunoglobulin family are also encompassed by the present invention. Both variable regions from specific antibodies are covered within the present invention, including complementarity determining regions (CDRs), as are antibody neutralizing regions, including those that bind effector molecules such as Fc regions. Antigen specific-encoding regions from antibodies, such as variable regions from IgGs, IgMs, or IgAs, can be employed with the protamine molecule complexed to the vector of the present invention in combination with an antibody neutralization region or with one of the therapeutic compounds described above.

In yet another embodiment, one gene may comprise a single-chain antibody. Methods for the production of single-chain antibodies are well known to those of skill in the art. The skilled artisan is referred to U.S. Pat. No. 5,359,046, (incorporated herein by reference) for such methods. A single chain antibody is created by fusing together the variable domains of the heavy and light chains using a short peptide linker, thereby reconstituting an antigen binding site on a single molecule.

Single-chain antibody variable fragments (scFvs) in which the C-terminus of one variable domain is tethered to the N-terminus of the other via a 15 to 25 amino acid peptide or linker, have been developed without significantly disrupting antigen binding or specificity of the binding (Bedzyk et al., 1990; Chaudhary et al, 1990). These Fvs lack the constant regions (Fc) present in the heavy and light chains of the native antibody.

Antibodies to a wide variety of molecules are contemplated, such as oncogenes, cytokines, growth factors, hormones, enzymes, transcription factors or receptors. Also contemplated are secreted antibodies targeted against serum, angiogenic factors (VEGF/VPF; βFGF; αFGF; and others), coagulation factors, and endothelial antigens necessary for angiogenesis (i.e., V3 integrin). Specifically contemplated are growth factors such as transforming growth factor, fibroblast growth factor, and platelet derived growth factor (PDGF) and PDGF family members.

The present invention further embodies composition targeting specific pathogens through the use of antigen-specific sequences or targeting specific cell types, such as those expressing cell surface markers to identify the cell. Examples of such cell surface markers would include tumor-associated antigens or cell-type specific markers such as CD4 or CD8.

5. Regions Mediating Protein-Protein or Ligand-Receptor Interaction

The use of a region of a protein that mediates protein-protein interactions, including ligand-receptor interactions, also is contemplated by the present invention. This region could be used as an inhibitor or a competitor of a protein-protein interaction or as a specific targeting motif. Consequently, the invention covers using a polypeptide, such as a polypeptide having a binding domain, to recruit a protein region that mediates a protein-protein interaction to a cancer cell. Once the compositions of the present invention reach the cancer cell, more specific targeting of the composition is contemplated through the use of a region that mediates protein-protein interactions including ligand-receptor interactions.

Protein-protein interactions include interactions between and among proteins such as receptors and ligands; receptors and receptors; polymeric complexes; transcription factors; kinases and downstream targets; enzymes and substrates; etc. For example, a ligand binding domain mediates the protein:protein interaction between a ligand and its cognate receptor. Consequently, this domain could be used either to inhibit or compete with endogenous ligand binding or to target more specifically cell types that express a receptor that recognizes the ligand binding domain operatively attached to the protamine molecule or the therapeutic molecule.

Examples of ligand binding domains include ligands such as VEGF/VPF; βPFGF; αFGF; coagulation factors, and endothelial antigens necessary for angiogenesis (i.e., V3 integrin); growth factors such as transforming growth factor, fibroblast growth factor, colony stimulating factor, Kit ligand (KL), flk-2/flt-3, and platelet derived growth factor (PDGF) and PDGF family members; ligands that bind to cell surface receptors such as MHC molecules, among other.

The most extensively characterized ligands are asialoorosomucoid (ASOR) (Wu and Wu, 1987) and transferrin (Wagner et al., 1990). Recently, a synthetic neoglycoprotein, which recognizes the same receptor as ASOR, has been used as a gene delivery vehicle (Ferkol et al., 1993; Perales et al., 1994) and epidermal growth factor (EGF) has also been used to deliver genes to squamous carcinoma cells (Myers, EPO 0273085).

In other embodiments, Nicolau et al. (1987) employed lactosyl-ceramide, a galactose-terminal asialganglioside, incorporated into liposomes and observed an increase in the uptake of the insulin gene by hepatocytes. Also, the human prostate-specific antigen (Watt et al, 1986) may be used as the receptor for mediated delivery to prostate tissue.

6. Cytokines

Another class of compounds that is contemplated to be operatively linked to a vector complexed to at least one protamine molecule or to a protamine molecule of the present invention includes interleukins and cytokines, such as interleukin 1 (IL-1), IL-2, IL-3, IL-4, IL-5, IL-6, IL-7, IL-8, IL-9, IL-10, IL-11, IL-12, IL-13, IL-14, IL-15, β-interferon, α-interferon, γ-interferon, angiostatin, thrombospondin, endostatin, METH-1, METH-2, Flk2/Flt3 ligand, GM-CSF, G-CSF, M-CSF, and tumor necrosis factor (TNF).

7. Growth Factors

In other embodiments of the present invention, growth factors or ligands will be encompassed by the therapeutic agent. Examples include VEGF/VPF, FGF, TGFβ, ligands that bind to a TIE, tumor-associated fibronectin isoforms, scatter factor, hepatocyte growth factor, fibroblast growth factor, platelet factor (PF4), PDGF, KIT ligand (KL), colony stimulating factors (CSFs), LIF, and TIMP.

8. Hormones

Additional embodiments embrace the use of a hormone as a selective agent. For example, the following hormones or steroids can be implemented in the present invention: prednisone, progesterone, estrogen, androgen, gonadotropin, ACTH, CGH, or gastrointestinal hormones such as secretin.

9. Toxins

In certain embodiments of the present invention, therapeutic agents will include generally a plant-, fungus-, or bacteria-derived toxin such as ricin A-chain (Burbage, 1997), a ribosome inactivating protein, α-sarcin, aspergillin, restrictocin, a ribonuclease, diphtheria toxin A (Masuda et al., 1997; Lidor, 1997), pertussis toxin A subunit, E. coli enterotoxin toxin A subunit, cholera toxin A subunit, and pseudomonas toxin c-terminal. Recently, it was demonstrated that transfection of a plasmid containing a fusion protein regulatable diphtheria toxin A chain gene was cytotoxic for cancer cells. Thus, gene transfer of regulated toxin genes might also be applied to the treatment of diseases (Masuda et al., 1997).

10. Antisense Constructs

Antisense methodology takes advantage of the fact that nucleic acids tend to pair with “complementary” sequences. By complementary, it is meant that polynucleotides are those which are capable of base-pairing according to the standard Watson-Crick complementarity rules. That is, the larger purines will base pair with the smaller pyrimidines to form combinations of guanine paired with cytosine (G:C) and adenine paired with either thymine (A:T) in the case of DNA, or adenine paired with uracil (A:U) in the case of RNA. Inclusion of less common bases such as inosine, 5-methylcytosine, 6-methyladenine, hypoxanthine and others in hybridizing sequences does not interfere with pairing.

Targeting double-stranded (ds) DNA with polynucleotides leads to triple-helix formation; targeting RNA will lead to double-helix formation. Antisense polynucleotides, when introduced into a target cell, specifically bind to their target polynucleotide and interfere with transcription, RNA processing, transport, translation and/or stability. Antisense RNA constructs, or DNA encoding such antisense RNA's, may be employed to inhibit gene transcription or translation or both within a host cell, either in vitro or in vivo, such as within a host animal, including a human subject.

Antisense constructs may be designed to bind to the promoter and other control regions, exons, introns or even exon-intron boundaries of a gene. It is contemplated that the most effective antisense constructs will include regions complementary to intron/exon splice junctions. Thus, it is proposed that a preferred embodiment includes an antisense construct with complementarity to regions within 50-200 bases of an intron-exon splice junction. It has been observed that some exon sequences can be included in the construct without seriously affecting the target selectivity thereof. The amount of exonic material included will vary depending on the particular exon and intron sequences used. One can readily test whether too much exon DNA is included simply by testing the constructs in vitro to determine whether normal cellular function is affected or whether the expression of related genes having complementary sequences is altered.

As stated above, “antisense” means polynucleotide sequences that are substantially complementary over their entire length and have very few base mismatches. For example, sequences of fifteen bases in length may be termed complementary when they have complementary nucleotides at thirteen or fourteen positions. Naturally, sequences which are completely complementary will be sequences which are entirely complementary throughout their entire length and have no base mismatches. Other sequences with lower degrees of homology also are contemplated. For example, an antisense construct that has limited regions of high homology, but also contains a non-homologous region (e.g., ribozyme; see below) could be designed. These molecules, though having less than 50% homology, would bind to target sequences under appropriate conditions.

It may be advantageous to combine portions of genomic DNA with cDNA or synthetic sequences to generate specific constructs. For example, where an intron is desired in the ultimate construct, a genomic clone will need to be used. The cDNA or a synthesized polynucleotide may provide more convenient restriction sites for the remaining portion of the construct and, therefore, would be used for the rest of the sequence.

Particular oncogenes that are targets for antisense constructs are ras, myc, neu, raf, erb, src, fms, jun, trk, ret, hst, gsp, bcl-2, and abl. Also contemplated to be useful are anti-apoptotic genes and angiogenesis promoters. Other antisense constructs can be directed at genes encoding viral or microbial genes to reduce or eliminate pathogenicity. Specific constructs target genes such as viral env, pol, gag, rev, tat or coat or capsid genes, or microbial endotoxin, recombination, replication, or transcription genes.

11. Ribozymes

Although proteins traditionally have been used for catalysis of nucleic acids, another class of macromolecules has emerged as useful in this endeavor. Ribozymes are RNA-protein complexes that cleave nucleic acids in a site-specific fashion. Ribozymes have specific catalytic domains that possess endonuclease activity (Kim and Cook, 1987; Gerlach et al., 1987; Forster and Symons, 1987). For example, a large number of ribozymes accelerate phosphoester transfer reactions with a high degree of specificity, often cleaving only one of several phosphoesters in an oligonucleotide substrate (Michel and Westhof, 1990; Reinhold-Hurek and Shub, 1992). This specificity has been attributed to the requirement that the substrate bind via specific base-pairing interactions to the internal guide sequence (“IGS”) of the ribozyme prior to chemical reaction. Molecules for use as antisense constructs are also contemplated for use as ribozymes, and vice versa.

12. Chemo- and Radiotherapeutics

According to the invention, chemotherapeutic and radiotherapeutic compounds can be operatively attached to a vector complexed to at least one protamine molecule or to a protamine molecule of the present invention. Chemotherapeutic agents contemplated to be of use include, e.g., adriamycin, bleomycin, 5-fluorouracil (5FU), etoposide (VP-16), camptothecin, actinomycin-D, mitomycin C, cisplatin (CDDP), podophyllotoxin, verapamil, and even hydrogen peroxide.

13. Transcription Factors and Regulators

Another class of genes that can be applied in an advantageous combination are transcription factors, both negative and positive regulators. Examples include C/EBPα, IκB, NFκB, AP-1, YY-1, Sp1, CREB, VP16, and Par-4.

14. Cell Cycle Regulators

Cell cycle regulators provide possible advantages, when combined with other genes. Such cell cycle regulators include p27, p16, p21, p57, p18, p73, p19, p15, E2F-1, E2F-2, E2F-3, p107, p130, and E2F-4. Other cell cycle regulators include anti-angiogenic proteins, such as soluble Flk1 (dominant negative soluble VEGF receptor), soluble Wnt receptors, soluble Tie2/Tek receptor, soluble hemopexin domain of matrix metalloprotease 2, and soluble receptors of other angiogenic cytokines (e.g., VEGFR1, VEGFR2/KDR, VEGFR3/Flt4, and neutropilin-1 and -2 coreceptors).

15. Chemokines

Chemokines also may be used in the present invention. Chemokines generally act as chemoattractants to recruit immune effector cells to the site of chemokine expression. It may be advantageous to express a particular chemokine gene in combination with, for example, a cytokine gene, to enhance the recruitment of other immune system components to the site of treatment. Such chemokines include RANTES, MCAF, MIP1-alpha, MIP1-beta, and IP-10. The skilled artisan will recognize that certain cytokines are also known to have chemoattractant effects and could also be classified under the term chemokines.

16. Inducers of Apoptosis

Inducers of apoptosis, such as Bax, Bak, Bcl-Xₛ, Bad, Bim, Bik, Bid, Harakiri, Ad E1B, MDA7 and ICE-CED3 proteases, similarly could be of use according to the present invention.

Moreover, it should be reiterated that any of the agents listed here also can be used individually to treat the related condition in conjunction with providing a viral composition of the present invention to treat a malignancy.

B. Peptides and/or Polypeptides

Embodiments of the invention include a protamine molecule operatively linked or conjugated to a targeting moiety. The targeting moiety can include a peptide or polypeptide. A peptide or polypeptide may be a ligand for a cell surface receptor. The peptides of the invention can be synthesized in solution or on a solid support in accordance with conventional techniques. Various automatic synthesizers are commercially available and can be used in accordance with known protocols. See, for example, Stewart and Young, (1984); Tam et al., (1983); Merrifield, (1986); and Barany and Merrifield (1979), each incorporated herein by reference. Short peptide sequences, or libraries of overlapping peptides, usually from about 6 up to about 35 to 50 amino acids, which correspond to the selected regions described herein, can be readily synthesized and then screened in screening assays designed to identify reactive peptides. Peptides with at least about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 50, 55, 60, 65, 70, 75, 80, 85, 90, 95 or up to about 100 amino acid residues are contemplated by the present invention.

The viral compositions of the invention may include a peptide comprising a protamine peptide that has been modified to render it biologically protected. Biologically protected peptides have certain advantages over unprotected peptides when administered to human subjects and, as disclosed in U.S. Pat. No. 5,028,592, incorporated herein by reference, protected peptides often exhibit increased pharmacological activity. Further, the viral compositions of the present invention may comprise a ligand that is covalently attached to the protamine by way of a linking moiety. The ligand is a polypeptide that may also be modified to render it biologically protected.

Compositions for use in the present invention may also comprise peptides that include all L-amino acids, all D-amino acids, or a mixture thereof. The use of D-amino acids may confer additional resistance to proteases naturally found within the human body and are less immunogenic and can therefore be expected to have longer biological half lives.

1. Linkers/Coupling Agents

If desired, dimers or multimers of the protamine molecule and the therapeutic or preventative compound may be joined via a biologically-releasable bond, such as a selectively-cleavable linker or amino acid sequence. For example, peptide linkers that include a cleavage site for an enzyme preferentially located or active within a tumor environment are contemplated. Exemplary forms of such peptide linkers are those that are cleaved by urokinase, plasmin, thrombin, Factor IXa, Factor Xa, or a metalloproteinase, such as collagenase, gelatinase, or stromelysin.

It is also contemplated that a peptide containing multimers of the protamine molecule may be comprised of heteromeric sequences, in which the binding sequences utilized are not identical to each other, or homomeric sequences, in which a binding domain sequence is repeated at least once. Amino acids such as selectively-cleavable linkers, synthetic linkers, or other amino acid sequences may be used to separate a binding domain from another binding domain. Alternatively, linker sequences may be employed both between at least once set of binding domains, as well as between a binding domain and a selective agent or compound. The term “binding domain” refers to at least one amino acid residue that is employed to link, conjugate, coordinate, or complex another compound or molecule, either directly (i.e., covalent bond) or indirectly (i.e., via a linking moiety).

Additionally, while numerous types of disulfide-bond containing linkers are known which can successfully be employed to conjugate the polypeptide having a therapeutic activity with the protamine molecule of the invention, certain linkers will generally be preferred over other linkers, based on differing pharmacologic characteristics and capabilities. For example, linkers that contain a disulfide bond that is sterically “hindered” are preferred, due to their greater stability in vivo, thus preventing release of the toxin moiety prior to binding at the site of action. Furthermore, while certain advantages in accordance with the invention will be realized through the use of any of a number of linking moieties, the inventors have found that the use of salicylhydroxamic acid will provide particular benefits. It is also contemplated that linkers are employed to conjugate the tumor suppression gene with selective agents to, for example, aid in detection.

2. Biochemical Cross-Linkers

The joining of any of the above components, to the protamine molecule will generally employ the same technology as developed for the preparation of an immunotoxin. It can be considered as a general guideline that any biochemical cross-linker that is appropriate for use in an immunotoxin will also be of use in the present context, and additional linkers may also be considered.

Cross-linking reagents are used to form molecular bridges that tie together functional groups of two different molecules, e.g., a stablizing and coagulating agent. To link two different proteins in a step-wise manner, hetero-bifunctional cross-linkers can be used that eliminate unwanted homopolymer formation. Non-limiting examples of hetero-bifunctional cross-linkers are listed in Table 3.

TABLE 3
HETERO-BIFUNCTIONAL CROSS-LINKERS

| | | | Spacer Arm |
|---|---|---|---|
| | | Advantages | Length\after |
| Linker | Reactive Toward | and Applications | crosslinking |
| SMPT | Primary amines | Greater stability | 11.2 A |
| | Sulfhydryls | | |
| SPDP | Primary amines | Thiolation | 6.8 A |
| | Sulfhydryls | Cleavable cross-linking | |
| LC-SPDP | Primary amines | Extended spacer arm | 15.6 A |
| | Sulfhydryls | | |
| Sulfo-LC- | Primary amines | Extended spacer arm | 15.6 A |
| SPDP | Sulfhydryls | Water-soluble | |
| SMCC | Primary amines | Stable maleimide | 11.6 A |
| | Sulfhydryls | reactive group Enzyme- | |
| | | antibody conjugation | |
| | | Hapten-carrier protein | |
| | | conjugation | |
| Sulfo-SMCC | Primary amines | Stable maleimide | 11.6 A |
| | Sulfhydryls | reactive group | |
| | | Water-soluble | |
| | | Enzyme-antibody | |
| | | conjugation | |
| MBS | Primary amines | Enzyme-antibody | 9.9 A |
| | Sulfhydryls | conjugation | |
| | | Hapten-carrier protein | |
| | | conjugation | |
| Sulfo-MBS | Primary amines | Water-soluble | 9.9 A |
| | Sulfhydryls | | |
| SIAB | Primary amines | Enzyme-antibody | 10.6 A |
| | Sulfhydryls | conjugation | |
| Sulfo-SIAB | Primary amines | Water-soluble | 10.6 A |
| | Sulfhydryls | | |
| SMPB | Primary amines | Extended spacer arm | 14.5 A |
| | Sulfhydryls | Enzyme-antibody | |
| | | conjugation | |
| Sulfo-SMPB | Primary amines | Extended spacer arm | 14.5 A |
| | Sulfhydryls | Water-soluble | |
| EDC/Sulfo- | Primary amines | Hapten-Carrier | 0 |
| NHS | Carboxyl groups | conjugation | |
| ABH | Carbohydrates | Reacts with sugar groups | 11.9 A |
| | Nonselective | | |

An exemplary hetero-bifunctional cross-linker contains two reactive groups: one reacting with primary amine group (e.g., N-hydroxy succinimide) and the other reacting with a thiol group (e.g., pyridyl disulfide, maleimides, halogens, etc.). Through the primary amine reactive group, the cross-linker may react with the lysine residue(s) of one protein (e.g., the selected antibody or fragment) and through the thiol reactive group, the cross-linker, already tied up to the first protein, reacts with the cysteine residue (free sulfhydryl group) of the other protein (e.g., the selective agent).

It can therefore be seen that a targeted peptide composition will generally have, or be derivatized to have, a functional group available for cross-linking purposes. This requirement is not considered to be limiting in that a wide variety of groups can be used in this manner. For example, primary or secondary amine groups, hydrazide or hydrazine groups, carboxyl alcohol, phosphate, or alkylating groups may be used for binding or cross-linking. For a general overview of linking technology, one may wish to refer to Ghose & Blair (1987).

The spacer arm between the two reactive groups of a cross-linkers may have various length and chemical compositions. A longer spacer arm allows a better flexibility of the conjugate components while some particular components in the bridge (e.g., benzene group) may lend extra stability to the reactive group or an increased resistance of the chemical link to the action of various aspects (e.g., disulfide bond resistant to reducing agents). The use of peptide spacers, such as L-Leu-L-Ala-L-Leu-L-Ala, is also contemplated.

It is preferred that a cross-linker having reasonable stability in blood will be employed. Numerous types of disulfide-bond containing linkers are known that can be successfully employed to conjugate targeting and therapeutic/preventative agents. Linkers that contain a disulfide bond that is sterically hindered may prove to give greater stability in vivo, preventing release of the targeting peptide prior to reaching the site of action. These linkers are thus one group of linking agents.

Another cross-linking reagent is SMPT, which is a bifunctional cross-linker containing a disulfide bond that is “sterically hindered” by an adjacent benzene ring and methyl groups. It is believed that steric hindrance of the disulfide bond serves a function of protecting the bond from attack by thiolate anions such as glutathione which can be present in tissues and blood, and thereby help in preventing decoupling of the conjugate prior to the delivery of the attached agent to the tumor site. It is contemplated that the SMPT agent may also be used in connection with the bispecific coagulating ligands of this invention.

The SMPT cross-linking reagent, as with many other known cross-linking reagents, lends the ability to cross-link functional groups such as the SH of cysteine or primary amines (e.g., the epsilon amino group of lysine). Another possible type of cross-linker includes the hetero-bifunctional photoreactive phenylazides containing a cleavable disulfide bond such as sulfosuccinimidyl-2-(p-azido salicylamido) ethyl-1,3′-dithiopropionate. The N-hydroxy-succinimidyl group reacts with primary amino groups and the phenylazide (upon photolysis) reacts non-selectively with any amino acid residue.

In addition to hindered cross-linkers, non-hindered linkers also can be employed in accordance herewith. Other useful cross-linkers, not considered to contain or generate a protected disulfide, include SATA, SPDP and 2-iminothiolane (Wawrzynczak & Thorpe, 1987). The use of such cross-linkers is well understood in the art.

Once conjugated, the targeting peptide generally will be purified to separate the conjugate from unconjugated targeting agents or coagulants and from other contaminants. A large a number of purification techniques are available for use in providing conjugates of a sufficient degree of purity to render them clinically useful. Purification methods based upon size separation, such as gel filtration, gel permeation or high performance liquid chromatography, will generally be of most use. Other chromatographic techniques, such as Blue-Sepharose separation, may also be used.

In addition to chemical conjugation, a purified protamine protein or peptide may be modified at the protein level. Included within the scope of the invention are protamine protein fragments or other derivatives or analogs that are differentially modified during or after translation, for example by glycosylation, acetylation, phosphorylation, amidation, derivatization by known protecting/blocking groups, and proteolytic cleavage. Any number of chemical modifications may be carried out by known techniques, including but not limited to specific chemical cleavage by cyanogen bromide, trypsin, chymotrypsin, papain, V8 protease, NaBH₄; acetylation, formylation, farnesylation, oxidation, reduction; metabolic synthesis in the presence of tunicamycin.

As will be understood by those of skill in the art, small modification and changes may be made in the structure of a domain that binds protamine to the viral vector or protamine to, for example, a ligand, including those changes that confer a greater binding affinity. Furthermore, certain amino acids may be substituted for other amino acids in a protein structure without appreciable loss of interactive binding capacity with the protamine or the therapeutic molecule. Since it is the interactive capacity and nature of a protein that defines that protein's biological functional activity, certain amino acid sequence substitutions can be made in a protein sequence (or, of course, its underlying DNA coding sequence) and nevertheless obtain a protein with like (agonistic) properties. It is thus contemplated by the inventors that various changes may be made in the binding sequence of therapeutic or preventative compound polypeptides or peptides (or underlying DNA) without appreciable loss of their biological utility or activity.

In the present invention, residues shown to be necessary for binding a polypeptide having a therapeutic activity or a protamine molecule generally should be substituted with conservative amino acids or not changed at all.

Amino acid substitutions are generally based on the relative similarity of the amino acid side-chain substituents, for example, their hydrophobicity, hydrophilicity, charge, size, and the like. An analysis of the size, shape, and type of the amino acid side-chain substituents reveals that arginine, lysine, and histidine are all positively charged residues; that alanine, glycine, and serine are all a similar size; and that phenylalanine, tryptophan, and tyrosine all have a generally similar shape. Therefore, based upon these considerations, the following subsets are defined herein as biologically functional equivalents: arginine, lysine, and histidine; alanine, glycine, and serine; and phenylalanine, tryptophan, and tyrosine.

To effect more quantitative changes, the hydropathic index of amino acids may be considered. Each amino acid has been assigned a hydropathic index on the basis of their hydrophobicity and charge characteristics, these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5), asparagine (−3.5); lysine (−3.9); and arginine (−4.5).

The importance of the hydropathic amino acid index in conferring interactive biological function on a protein is generally understood in the art (Kyte & Doolittle, 1982, incorporated herein by reference). It is known that certain amino acids may be substituted for other amino acids having a similar hydropathic index or score and still retain a similar biological activity. In making changes based upon the hydropathic index, the substitution of amino acids whose hydropathic indices are within ±2 is preferred, those which are within ±1 are particularly preferred, and those within ±0.5 are even more particularly preferred.

It also is understood in the art that the substitution of like amino acids can be made effectively on the basis of hydrophilicity, particularly where the biological functional equivalent protein or peptide thereby created is intended for use in immunological embodiments, as in the present case. U.S. Pat. No. 4,554,101, incorporated herein by reference, states that the greatest local average hydrophilicity of a protein, as governed by the hydrophilicity of its adjacent amino acids, correlates with its immunogenicity and antigenicity, i.e. with a biological property of the protein.

As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4).

In making changes based upon similar hydrophilicity values, the substitution of amino acids whose hydrophilicity values are within ±2 is preferred, those which are within ±1 are particularly preferred, and those within ±0.5 are even more particularly preferred.

It also is conceivable that non-peptide structures such as “peptide mimetics” may be used to duplicate the structure and contact points within the protamine-peptide or polypeptide conjugate structure.

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Materials and Methods

Cell Lines

Human lung cancer cell lines with varied p53 and 3p21.3 status were examined for the tumor-suppressing function of 3p genes in vitro and in vivo. One of these lines is H1299, a NSCLC cell line that contains an internal homozygous deletion of p53 and does not have a normal copy of chromosome 3 with a LOH of 3p alleles. Also, H1299 has very high levels of telomerase expression and activity. A549, is a lung carcinoma cell line that contains wild-type p53 with abnormal 3p alleles; H358 is a lung cancer cell line that contains wild-type p53 with 2 3p alleles; and H460 is, a lung cancer cell line that contains wild-type p53 with loss of one allele of the 3p21.3 region. Normal HBECs or fibroblast cells (Clonetics Inc., Walkersville, Md.) were also used to evaluate the general toxicity of the 3p genes and Ad-3 ps. The 293 cell line was used in the construction, amplification, and titration of adenoviral vectors. Cells were maintained in Quebecois Modified Eagle Medium (DMEM) containing 4.5 g/L of glucose with 10% FBS.

Construction of Recombinant Adenoviral Vectors

The recombinant adenoviral vectors were constructed using a recently developed ligation-mediated plasmid-adenovirus vector construction system. The gene of interest, e.g., a 3p gene, was first placed in a plasmid shuttle vector (pLJ37) containing the adenoviral inverted repeated terminal (IRT) sequence, an expression cassette of a cytomegalovirus (CMV) promoter and bovine growth hormone (BGH) poly (A) signal sequence, and having two unique restriction sites BstBI and ClaI at the 5′ and 3′ ends of the IRT-CMV-multiple cloning sites-BGH sequence, respectively. The BstBI/ClaI-released DNA fragment containing IRT-CMV-3p-BGH was then inserted into an adenoviral plasmid vector, pLJ34, which contains a complete E1 and E3-deleted adenovirus type 5 genome and three unique restriction sites (PacI, BstBI, and ClaI), by in vitro ligation using BstBI and ClaI sites. After transformation into E. coli, >80% of the transformants had the correct insert. Finally, PacI/BstBI digestion of the resulting plasmid allows release of the entire adenovirus genome-containing the 3p gene.

The recombinant Ad-3p DNA was then transfected into 293 cells, resulting in a homogeneous population of recombinant Ad-3p. Other adenoviral vectors Ad-p53, Ad-LacZ, Ad-GFP, Ad-MDA7, Ad-EV, Ad-FHIT were prepared by conventional methods and obtained from adenoviral stocks prepared by Adenoviral Vector Core at MDACC. Ad-E1- (Ad-EV), an empty E1- vector, was used as a negative control. Control vectors were obtained from the Adenoviral Vector Core at the University of Texas M.D. Anderson Cancer Center. Viral titers were determined by both optical density measurement and plaque assay.

DNA Sequencing and Analysis

Potential contamination of the viral preparation by the wild-type virus was monitored by polymerase chain reaction (PCR) analysis. Sequences of 3p genes in the viral vectors were confirmed by automated DNA sequencing.

Preparation of Protamine-Adenoviral Vector Complex

The protamine-adenovirus complexes were prepared by mixing about 10-20 mL of original stock without dilution, which provided about 1×10¹⁰ viral particles, with 50 μg of protamine sulfate (10 mg/ml)(Fujisawa USA, Inc., Deerfield, Ill.). The mixture was incubated for 10 min at ambient temperature to form the complex, then diluted in an appropriate volume of PBS for designated in vitro or in vivo studies. See, FIG. 1 for an illustration of an exemplary protamine-adenovirus complex.

Preparation and Administration of Protamine-Adenoviral Complex In Vitro

The adenovirus stock and reagents are incubated for at least 15 min at ambient temperature. The adenoviral vector stock was then diluted in a final concentration of 1×10¹⁰ viral particles/50 μl in PBS. The protamine sulfate solution was diluted to a final concentration of 100 μg/50 μl in PBS. The diluted viral vector was then mixed with the diluted protamine by gentle aspiration, and then incubated for 10-15 min at ambient temperature to form the composition comprising the protamine-adenovirus complex.

It was observed that as the resistance to adenoviral transduction increased for a cell line, the transduction efficient to the protamine-adenovirus complex increased.

Preparation and Administration of Protamine-Adenoviral vector Complex In Vivo

A solution of 3×10¹⁰ viral particles in PBS was diluted to a final volume of 50 μl. The protamine solution was diluted to a final concentration of 300 μg/50 μl in PBS. The diluted viral vector solution was then mixed with the diluted protamine by gentle aspiration, and then incubated for 10-15 min at ambient temperature to form the viral composition comprising a protamine-adenovirus complex.

About 100 μl of D5W was added to the protamine-adenovirus complex solution and gently mixed. Injection of the viral composition in D5W (200 μ1/mouse) using a 32-gauge needle was performed slowly (within about 1-2 min) via intravenous injection or locally to the tumor (200 μl/tumor).

Preparation and Administration of Protamine-Adenoviral vector Complex for Nebulization

About 5×10¹¹ viral particles in PBS were diluted to a final volume of 500 μl, and then mixed with 500 μl (5 mg) of protamine. The mixture was incubated for 10-15 min at ambient temperature to form the viral composition comprising the protamine-adenovirus complex. The viral composition (1 mL) was diluted to a final volume of 5 ml in PBS just before application.

The diluted viral composition was placed into a nebulizer chamber, which was then closed tightly. The nebulizer was fixed into the aerosol application unit, and mice (up to 10) were placed into the aerosol administration unit. After tightly sealing the aerosol administration unit, the aerosol compressor was turned on. The mice were treated by respiratory inhalation with the entire volume (5 ml) of the viral composition, which took about 20-30 min.

All working surfaces and aerosol administration units were disinfected after treatment.

### Example 2

### Effects of Ad-TSGs on Tumor Cell Growth and Proliferation

The growth properties of various lung cancer cells with abnormalities of various tumor suppressor genes (TSGs) were tested for alteration by the introduction of wild-type TSGs. Cell viability in Ad-TSG-transduced tumor cells at varied MOIs at designated posttransduction time intervals were assayed by XTT staining (Roche Molecular Biochemicals, Mannheim, Germany). The untransduced and Ad-EV-, Ad-GFP-, or Ad-LacZ-transduced cells were used as controls. Each experiment was repeated at least three times, with each treatment in duplicate or triplicate.

Proliferation of the Ad-TSG-transduced cells was analyzed by an immunofluorescence-enzyme-linked immunosorbent assay for incorporation of bromodeoxyuridine (BrdU) into cellular DNA in the 96-well plates following manufacturers instructions (Roche Molecular Biochemicals). Ad-3p-transduced normal HBECs were used to evaluate the possible general toxicity of the TSGs and Ad-TSGs in vitro. Transcription and expression of TSGs in Ad-TSG-transduced cells were examined by reverse transcriptase-polymerase chain reaction, northern- and/or western-blot analysis with anti-TSG protein polyclonal antibodies, which were obtained from commercial resources or from collaborators.

### Example 3

### Induction of Apoptosis and Alteration of Cell Cycle Kinetics by TSGs

Inhibition of tumor cell growth and proliferation by tumor suppressor genes is commonly characterized by induction of apoptosis and alteration of cell cycle processes. TSG-induced apoptosis and cell cycle kinetics were analyzed by flow cytometry using the terminal deoxy transferase deoxyuridine triphosphate (dUTP) nick-end labelling (TUNEL) reaction with fluorescein isothiocyanate-labeled dUTP (Roche Molecular Biochemicals) and propidium iodide staining, respectively. Cells (1×10⁶/well) are seeded on six-well plates and transduced with Ad-TSG constructs; untreated and Ad-EV-, Ad-GFP-, or Ad-LacZ-transduced cells were used as controls. Cells were harvested at designated post-transduction times and then analyzed for DNA fragmentation and apoptosis by TUNEL reaction, and for DNA content and cell cycle status by propidium iodide staining using flow cytometry.

### Example 4

### Effects of Gene Expression on Tumorigenicity and Tumor Growth In Vivo

For the tumorigenicity study, H1299 or A549 cells were transduced in vitro with Ad-TSGs at an appropriate MOI with phosphate-buffered saline (PBS) alone as a mock control, Ad-EV as a negative control, and Ad-LacZ as a nonspecific control. The transduced cells were harvested at 24 h and 48 h post-transduction. The viability of the cells was determined by trypan blue exclusion staining. Viable cells (1×10⁷) were then injected subcutaneously into the right flank of 6- to 8-week-old female nude mice. Tumor formation in mice was observed two or three times weekly for up to 3 months. Tumor dimensions were measured every 2 or 3 days.

### Example 5

### Effect of 3p Genes on Tumor Growth

H1299 or A549 cells were used to establish subcutaneous tumors in nude mice. Briefly, 1×10⁷ cells were injected into the right flank of 6- to 8-week-old female nude mice. When the tumors reached 5 to 10 mm in diameter (at about 2 weeks postinjection), the animals were intratumorally injected with Ad-TSGs and control vectors, respectively, 4 to 5 times within 10 to 12 days for at a total dose of 3 to 5×10¹⁰ pfu per tumor. Tumor size was measured and calculated as described above. At the end of each experiment, the animals were killed and the tumors were excised and processed for pathological and immunohistochemical analysis.

### Example 6

### Effect of TSGs on Metastatic Tumor Growth by P-Ad-TSG-Mediated Gene Transfer In Vivo

The experimental lung metastasis models of human NSCLC H1299 and A549 cells or pancreatic carcinoma S2-VP10 cells were used to study the effects of various TSGs on tumor progression and metastasis by systemic treatment of lung metastatic tumors using intravenous injection of P-Ad-TSG complexes. A549 cells (1-2×10⁶) in 200 ml PBS were intravenously inoculated into nude mice and H1299 cells (1-2×10⁶) into SCID mice. Metastatic tumor colonies were formed 7-10 days post-inoculation. P-Ad-TSGs and control complexes were administered to animals by i.v. injection every other two days for 3 times each at a dose of 2-5×10¹⁰ viral particles/200-500 mg protamine, in a total volume of 200 ml per animal. Animals were sacrificed two weeks after the last injection. Lung metastasis were stained with Indian ink 51, tumor colonies on the surfaces of lung were counted under an anatomic microscope, and then the lung tissue were sectioned for further pathologic and immunohistochemical analysis.

### Example 7

### Western Blot Analysis of Treated Cells

Expression of 3p genes in Ad-3p-transduced cells was analyzed by Western blot, using polyclonal antibodies against polypeptides derived from predicted 3p amino acid sequences or monoclonal antibodies against c-myc or FLAG tags in 3p fusion proteins. Cells grown in 60 mm-dishes (1-5×10⁶/well) were treated with Ad-3 ps, (PBS alone was used as a control). Each lane was loaded with about 60 μg cell lysate protein and electrophoresed at 100 V for 1-2 h on a SDS-PAGE gel. Proteins were then transferred from gels to Hybond-ECL membranes (Amersham International, England). Membranes were blocked in blocking solution (3% dry milk, 0.1% Tween 20 in PBS) for 1 h at room temperature. Membranes were then incubated with 1:1000 dilution of rabbit anti-human 3p peptides or anti-myc or FLAG monoclonal antibodies, and 1:1000 dilutions of mouse anti-β-actin monoclonal antibodies. Immunocomplexes were detected with secondary HRP-labeled rabbit anti-mouse IgG or goat anti-rabbit IgG antibodies using an ECL kit (Amersham), according to the manufacturer's instructions.

### Example 8

### Method of Neutralizing Antibody Assay

Either a C3H or C57BL6 mouse strain were used. Treatment and serum sample collection were performed at particular time points based on a schedule. The mice were divided into various treatment groups: Group I: PBS, Group II. Protamine (or Ca++/Phosphate), Group III. Ad-GFP, Group IV. Protamine-Ad-GFP (or Ca⁺⁺Phosphate-Ad-GFP), and Group V, Protamine-Ad-X (X, the gene of interest). The pre-immune serum (PI) was collected; followed by inoculation of the mice with each of the treatments. At 3 weeks post-inoculation (IM-1), serum was collected, followed by a repeat injection given at week 4.

Serum was collected 24 hr after the second inoculation with various treatment groups. The animals were sacrificed and lung and liver samples were collected for determination of GFP expression.

The assay for neutralizing antibodies in the collected serum was performed by first plating H1299 cells from 95% confluent of 100 mm dishes to a 96-well plate with 5×10³ cells/well which were incubated at 37° C. overnight. The samples were heat-inactivated for testing at 55° C. for 30 min.

Serial dilutions of the serum samples at 1:3 in 100 μl of growth medium were prepared and mixed with Ad-GFP. A serum of known titer was used as a positive control. Blanks comprised cells without serum or adenovirus.

The medium was removed from each well and 100 μl of above medium with various serum dilutions and Ad-GFP viral vectors were added to a corresponding well. The reaction was incubated at 37° C. for 24-48 hr. The medium was then removed and analyzed for fluorescence intensity using a fluorescence microplate reader at excitation wavelength of 485 nm and emission wavelength of 530 nm.

Data can be plotted using a linear regression curve fit to determine the titer of neutralizing antibody at ID50 (50% of fluorescence intensity reduction) from the fitted equation y=aX+b.

### Example 9

### Protamine Adenovirus Complex Inoculation of A549 Metastases in Nude Mice

A549 cells were grown in F12 medium with 5% serum and 5% glutamine till about 70% confluence. Mice were irradiated at 350 rad one day before injection of protamine-adenvirus complex. Cells were harvested and dilute in PBS at a final concentration of 1×10⁶ cells/100 μl PBS. Cells were injected into mice by the tail vein with 100 μl of 1×10⁶A549 cells/mouse

Intravenous (i.v.) or local injection in mice was carried out as follows. 1×10¹¹ viral particles were diluted in PBS to a final volume of 100 μl. Protamine was diluted to a final concentration of 150 μg/100 μl in D5W. Diluted viral vectors were mixed with the diluted protamine by pipetting up and down several times. The protamine-adenovirus complex was incubated for 10-15 min at RT

The protamine-adenovirus-D5W solution was injected at 200 μl/mouse via i.v. slowly (within about 1-2 min) with a 32-gauge needle, or locally to the tumor at 200 μL/tumor. The treatment schedule included i.v. injection on day 1, 7, 10, and 14.

Staining of metastatic tumors was done as follows. At the end of study animals were sacrificed by CO₂ inhalation. The chest of the mouse was immediately open to expose the trachea. About 2 ml of 15% black India ink (add several drops of Ammonium hydrate to maintain ink suspension) was injected through the trachea with 28 gauge needle. The lungs were removed and fix in Fekete's solution (100 ml of 70% ethanol, 10 ml of formalin, and 5 ml of glacial acetic acid). White nodules on the black lung surface are counted under a dissecting microscope.

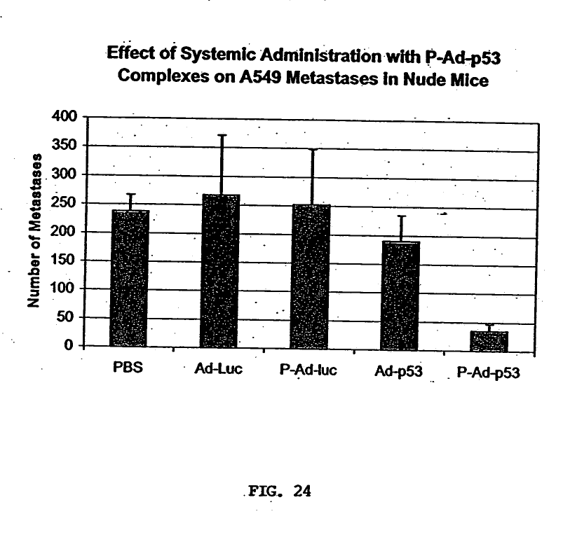

The results of this study are provided in FIG. 24. In summary protamine-conjugated Ad-p53 showed a significant inhibition on the development of lung metastases by systemic injection of the complexes compared to unconjugated Ad-p53 alone. The Ad-p53 alone and the control vectors, either Ad-Luc or P-Ad-Luc also showed no effect on the development of metastases, as expected, compared to PBS-protamine treated control group.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.

U.S. Pat. No. 3,791,932

U.S. Pat. No. 3,949,064

U.S. Pat. No. 4,174,384

U.S. Pat. No. 4,472,509

U.S. Pat. No. 4,554,101

U.S. Pat. No. 4,599,230

U.S. Pat. No. 4,599,231

U.S. Pat. No. 4,601,903

U.S. Pat. No. 4,608,251

U.S. Pat. No. 4,682,195

U.S. Pat. No. 4,683,202

U.S. Pat. No. 5,021,236

U.S. Pat. No. 5,028,592

U.S. Pat. No. 5,252,479

U.S. Pat. No. 5,359,046

U.S. Pat. No. 5,645,897

U.S. Pat. No. 5,672,344

U.S. Pat. No. 5,705,629

WO 00/05356

WO 00/26368

Alemany et al., 2000.

Anderson, Science, 256:808-13 (1992).

Arcasoy et al., Chest, 111: 142S-143S, 1997.

Arcasoy et al., Gene Ther., 4:32-38, 1997.

Arcone et al., Nucleic Acids Research, 16(8):3195-3207, 1988.

Arai et al., Proc. Natl Acad. Sci. USA 94: 13862-13867, 1997.

Barany and Merrifield, In: The Peptides: Analysis, Synthesis, Biology, Gross and Meinhofer, eds., Academic Press, New York, pp. 3-284, 1980.

Bartlett et al., Proc. Natl. Acad. Sci. USA, 93:8852-8857, 1996.

Batterson and Roizman, J. Virol., 46:371-377, 1983.

Bedzyk et al, J. Biol. Chem., 265:18615, 1990.

Bellon et al., de Ses Filiales,190(1):109-142, 1996.

Bems, Microbiol Rev, 54:316-29, 1990.

Berns and Bohenzky, Adv. Virus Res., 32:243-307, 1987.

Bems and Giraud, Curr. Top. Microbiol. Immunol., 218:1-23, 1996.

Bischoffet al, Science 274: 373-376, 1996.

Burbage et al, Leuk Res, 21(7):681-690, 1997.

Campbell et al., J. Mol. Biol., 180:1-19, 1984.

Chaudhary et al. Proc. Natl. Acad. Sci., 87:9491, 1990.

Coffin, In: Virology, Fields et al., eds., Raven Press, New York, pp. 1437-1500, 1990.

Couch et al., Am. Rev. Resp. Dis., 88:394-403, 1963.

DeLuca et al., J. Virol., 56:558-570, 1985.

Diller et al. Mol. Cell. Bio., 10: 5772-5781, 1990.

Doronin et al., J. Virol. 75: 3314-3324, 2001

Elroy-Stein et al., Proc. Nat'l Acad. Sci. USA, 1989.

Elshami et al., Gene Therapy, 7(2):141-148, 1996.

Fasbender et al., J. Biol.Chem., 272: 6479-6489, 1997.

Felzmann et al., Gene Ther. 4: 1322-1329, 1997.

Ferkol et al., FASEB J., 7:1081-1091, 1993.

Forster and Symons, Cell, 49:211-220, 1987.

French, et al., Circulation, 90(5):2414-2424, 1994.

Freshney, In: Animal Cell Culture: a Practical Approach, Second Edition, Oxford/New York, IRL Press, Oxford University Press, 1992.

Froehler et al., Nucleic Acids Res. 14(13):5399-5407, 1986.

Gerlach et al., Nature (London), 328:802-805, 1987.

Ghose & Blair, CRC Critical Reviews in Therapeutic Drug Carrier Systems, 3:262-359, 1987.

Ghosh-Choudhury et al., EMBO J, 6:1733-1739, 1987.

Ginsberg et al., PNAS, 88(5)1651-1655, 1991.

Glorioso et al., Ann. Rev. Microbiol. 49:675-710, 1995.

Goldman et al., Proc. Natl. Acad. Sci. USA, 95: 8795-8800, 1998.

Goldman and Wilson, J. Virol., 69: 5951-5958, 1995.

Gomez-Foix et al., J. Biol. Chem., 267:25129-25134, 1992.

Graham and Prevec, In: Methods in Molecular Biology: Gene Transfer and Expression Protocol, E. J. Murray, ed., Humana Press, Clifton, N.J., 7:109-128, 1991.

Graham and Prevec, Biotechnology, 20:363-390, 1992.

Graham et al, J. Gen. Virol., 36:59-72, 1977.

Grunhaus and Horwitz, Seminar in Virology, 3:237-252, 1992.

Harada and Berk, J. Viral 73 : 5333-5344, 1999.

Harlow and Lane, Antibodies: A Laboratory manual, Cold Spring Harbor Laboratory, 1988.

Heise et al., Nature Med. 6: 639-645, 1997.

Heise and Kim, J. Clin. Invest. 105, 847-851, 2000.

Hersdorffer et al., DNA Cell Biol., 9:713-723, 1990.

Herz and Gerard, Proc. Nat'l Acad. Sci. USA, 90:2812-2816, 1993.

Holland et al., Virology, 101:10-18, 1980.

Honess and Roizman, J. Virol., 14:8-19, 1974.

Honess and Roizman, J. Virol., 16:1308-1326, 1975.

Johnson et al., In: Biotechnology And Pharmacy, Pezzuto et al., eds., Chapman and Hall, New York, 1993.

Joki, et al., Human Gene Ther., 6:1507-1513, 1995.

Jones and Shenk, Cell, 13:181-188, 1978.

Kageyama, et al., J. Biol. Chem., 262(5):2345-2351, 1987.

Karlsson et al., EMBO J., 5:2377-2385, 1986.

Kearns, et al., Gene Ther., 3:748-755, 1996.

Kim and Cook, Proc. Natl. Acad. Sci. USA, 84:8788-8792, 1987.

Kotin and Berns, Virol., 170:460-467, 1989.

Kotin et al., Proc. Natl. Acad. Sci. USA, 87:2211-2215, 1990.

Kotin et al., Genomics, 10:831-834, 1991.

Kyte and Doolittle, J. Mol. Biol., 157:105-132, 1982.

Le Gal La Salle et al., Science, 259:988-990, 1993.

Lanuti et al., Gene Ther., 6:1600-1610, 1999.

Levrero et al., Gene, 101: 195-202, 1991.

Li and Huang, Gene Ther., 4: 891-900, 1997.

Li et al., Gene Ther., 5: 930-937, 1998.

Lidor et al., Am J Obstet Gynecol;177(3):579-585, 1997.

Lubeck et al., AIDS Res. Hum. Retroviruses, 10:1443-1449, 1994.

Macejak and Sarnow, Nature, 353:90-94, 1991.

Mann et al., Cell, 33:153-159, 1983.

Markowitz et al., J. Virol., 62:1120-1124, 1988.

Masuda et al., Proc Natl Acad Sci USA, 94(26):14701-14706, 1997.

Merrifield, Science, 232:341-7, 1986.

Michel and Westhof, J. Mol. Biol., 216:585-610, 1990.

Mizukami et al., Virology, 217:124-130, 1996.

Morein et al., Dev. Biol. Stand. 92:33-39, 1998.

Mostov et al., Cell 46:613-621, 1986.

Nicolas and Rubinstein, In: Vectors: A Survey of Molecular Cloning Vectors and Their Uses, Rodriguez and Denhardt, eds., Butterworth, Stoneham, England, pp. 494-513, 1988.

Nicolau et al., Methods Enzymol., 149:157-176, 1987.

Ogawa, W., Neuropathologica, 77(3):244-253, 1989.

Olivierio et al., EMBO J., 6(7):1905-1912, 1987.

Ostrove et al., Virology, 113:532-533, 1981.

Paskind et al., Virology, 67:242-248, 1975.

Pelletier and Sonenberg, Nature, 334:320-325, 1988.

Perales et al., Proc. Natl. Acad. Sci. 91:4086-4090, 1994.

Poli and Cortese, Proc. Natl. Acad. Sci. USA, 86:8202-8206, 1989.

Ponnazhagan, et al., J. Gen. Virol., 77:1111-1122, 1996.

Ponnazhagan, et al, Hum. Gene Ther., 8:275-284, 1997a.

Post et al., Cell 24:555-565, 1981.

Prowse et al., Mol Cell Biol, 8:42-51, 1988.

Putzer et al., Proc. Natl. Acad. Sci. USA 94: 10889-10894, 1997.

Racher et al., Biotechnology Techniques, 9:169-174, 1995.

Ragot et al., Nature, 361:647-650, 1993.

Reinhold-Hurek and Shub, Nature, 357:173-176, 1992.

Remington's Pharmaceutical Sciences, 15th ed., Mack Publishing Company, Easton, Pa., 1980.

Renan, Radiother. Oncol., 19:197-218, 1990.

Rich et al., Hum. Gene Ther., 4:461-476, 1993.

Rodriguez et al., 1997.

Roizman and Sears, In Fields' Virology, 3rd Edition, eds. Fields, et al. (Raven Press, New York, N.Y.), pp. 2231-2295, 1995.

Ron et al., Mol. Cell. Biol., 2887-2895, 1991.

Rosenfeld et al., Cell, 68: 143-155, 1992.

Rosenfeld et al., Science, 252:431-434, 1991.

Roux et al., Proc. Natl Acad. Sci. USA, 86:9079-9083, 1989.

Sambrook, et al., , Molecular Cloning, A Laboratory Manual, 2nd ed., Cold Spring Harbor Laboratory, Cold Spring, Harbor, N.Y., 1989.

Samulski et al., EMBO J., 10:3941-3950, 1991.

Shiraishi, et al., Transplant International, 1-0(3):202-206, 1997.

Smith and Moss, Gene 25:21-28, 1983.

Song et al., Hum. Gene. Ther., 8(10):1207-1217, 1997.

Stewart and Young, Solid Phase Peptide Synthesis, 2d. ed., Pierce Chemical Co., 1984.

Stratford-Perricaudet and Perricaudet, In: Human Gene Transfer, O. Cohen-Haguenauer and M. Boiron, eds., John Libbey Eurotext, France, p. 51-61, 1991.

Stratford-Perricaudet et al., Hum. Gene. Ther., 1:241-256, 1990.

Suzuki et al., 2001.

Tam et al., J. Am. Chem. Soc., 105:6442, 1983.

Temin, In: Gene Transfer, Kucherlapati, ed., Plenum Press, New York, pp. 149-188, 1986.

Top et al., J. Infect. Dis., 124:155-160, 1971.

Tollefson et al., J. Virol. 70: 2296-2306, 1996a.

Tollefson et al., Virol. 220: 152-162, 1996b.

Topfet al., Gene Ther. 5: 507-513, 1998.

Varmus et al., Cell, 25:23-36, 1981.

Wada et al., “Codon usage tabulated from the GenBank genetic sequence data,” Nucleic Acids Res. 18:2367-2411, 1990.

Wagner et al., Proc. Natl. Acad. Sci. 87(9):3410-3414, 1990.

Walther and Stein, J. Mol. Med, 74:379-392, 1996.

Watt et al., Proc. Natl Acad. Sci., 83(2): 3166-3170, 1986.

Wawrzynczak and Thorpe In: Immunoconjugates: Antibody Conjugates in Radioimaging and Therapy of Cancer, Vogel (ed.), New York, Oxford University Press, pp. 28-55, 1987.

Werthman et al., Journal of Urology, 155(2):753-756, 1996.

Wichham et al., Nat.Biotechnol., 14: 1570-1574, 1996.

Wilder et al., Gene Therapy 6: 57-62, 1999a.

Wilder et al., Cancer Res. 59: 410-413, 1999b.

Wilson, et al., Mol. Cell. Biol., 6181-6191, 1990.

Wu and Wu, J. Biol. Chem., 262:4429-4432, 1987.

Zechner, et al., Mol. Cell. Biol., 2394-2401, 1988.

What is claimed:

1. A method of treating cancer comprising administering to a cancer patient an effective amount of a viral composition comprising
i) a protamine molecule; and
ii) a therapeutic viral vector.

2. The method of claim 1, wherein the therapeutic viral vector is a viral vector comprising a nucleic acid encoding a tumor suppressor under the control of a promoter.

3. The method of claim 1, wherein the viral composition is in a pharmacologically acceptable solution.

4. The method of claim 1, wherein the viral composition comprises a ratio of about 10¹⁰ viral particles to about 100 μg protamine.

5. The method of claim 1, wherein the viral composition comprises a ratio of about 10¹⁰ viral particles to about 200 μg protamine.

6. The method of claim 1, wherein the viral composition comprises a ratio of about 10¹⁰ viral particles to about 300 μg protamine.

7. The method of claim 1, wherein the viral composition comprises a ratio of about 10¹¹ viral particles to about 100 μg protamine.

8. The method of claim 1, wherein the viral composition comprises a ratio of about 10¹¹ viral particles to about 200 μg protamine.

9. The method of claim 1, wherein the viral composition comprises a ratio of about 10¹¹ viral particles to about 300 μg protamine.

10. The method of claim 1, wherein the viral vector is an adenoviral vector, a retroviral vector, a vaccinia viral vector, an adeno-associated viral vector, a polyoma viral vector, or a herpes viral vector.

11. The method of claim 10, wherein the viral vector is an adenoviral vector.

12. The method of claim 11, wherein the adenoviral vector lacks the E1b coding region.

13. The method of claim 2, wherein the tumor suppressor is p53, FHIT, MDA7, or 3p21.3.

14. The method of claim 13, wherein the tumor suppressor is p53.

15. The method of claim 2, wherein the promoter is a CMV IE, dectin-1, dectin-2, human CD11c, F4/80, SM22 a MHC class II promoter, SV40, polyoma or adenovirus 2 promoter.

16. The method of claim 1, wherein between about 10¹⁰ to about 10¹⁵ viral particle are administered.

17. The method of claim 1, wherein the administration is by respiratory inhalation, intravenous injection, continuous infusion, aerosol inhalation, intratumoral injection or intravascular injection.

18. The method of claim 1, wherein the cancer is lung cancer, human lung cancer, non-small cell lung cancer, adenocarcinoma, epithelial cancer, soft tissue carcinoma, or Kaposi's sarcoma.

19. The method of claim 1, wherein the cancer comprises a tumor.

20. The method of claim 19, further comprising resecting all or part of the tumor.

21. The method of claim 20, wherein the tumor resection occurs prior to said administration.

22. The method of claim 21, wherein the administration comprises injection of the residual tumor site.

23. The method of claim 20, wherein the tumor resection is performed by bronchoscopy.

24. The method of claim 1, wherein the protamine further comprises a linking moiety.

25. The method of claim 24, wherein the linking moiety is SHA, SMTP, SPDP, LC-SPDP, Sulpho-LC-SDPD, SMCC, Sulfo-SMCC, MBS, Sulfo-MBS, SLAB, Sulfo-SIAB, SMPB, Sulfo-SMPB, EDC/Sulfo-NHS, or ABH.

26. The method of claim 25, wherein the linking moiety is salicylhydroxamic acid (SHA).

27. The method of claim 24, further comprising a targeting ligand coupled to the linking moiety.

28. The method of claim 27, wherein the targeting ligand is a polypeptide.

29. The method of claim 28, wherein the polypeptide is a ligand for a cell surface receptor.

30. The method of claim 1, wherein the viral composition comprises an adenovirus that is replication competent in one or more types of human neoplastic cells.

31. The viral composition of claim 30, wherein the adenovirus does not replicate in one or more non-neoplastic cells to the same extent that it replicates in neoplastic cells.

32. The viral composition of claim 30, wherein the adenovirus exhibits an upregulated expression of ADP relative to wild-type adenovirus.

33. A viral composition comprising:
a) a protamine molecule; and
b) an a therapeutic adenovirus.

34. The viral composition of claim 33, wherein the therapeutic adenovirus is an adenoviral vector comprising a nucleic acid encoding a tumor suppressor polypeptide wherein the composition is in a pharmaceutically acceptable composition.

35. The viral composition of claim 34, wherein the adenoviral vector comprises a wild type p53, an MDA7, an FHIT or a PTEN tumor suppressor polypeptide.

36. The viral composition of claim 35, wherein the tumor suppressor polypeptide is p53.

37. The viral composition of claim 33, wherein a linking moiety is covalently attached to the protamine molecule.

38. The viral composition of claim 37, wherein the linking moiety is SHA, SMTP, SPDP, LC-SPDP, Sulpho-LC-SDPD, SMCC, Sulfo-SMCC, MBS, Sulfo-MBS, SIAB, Sulfo-SIAB, SMPB, Sulfo-SMPB, EDC/Sulfo-NHS, or ABH.

39. The viral composition of claim 38, wherein the linking moiety is salicylhydroxamic acid (SHA).

40. The viral composition of claim 37, wherein the linking moiety is coupled to a polypeptide.

41. The viral composition of claim 40, wherein the polypeptide is a ligand for a cell surface receptor.

42. The viral composition of claim 33, wherein the viral composition comprises a ratio of about 10¹⁰-10¹⁵ viral particles to about 50-300 μg protamine.

43. The viral composition of claim 42, wherein the viral composition comprises a ratio of about 10¹¹-10¹³ viral particles to about 100-200 μg protamine.

44. The viral composition of claim 33, wherein the therapeutic adenovirus is an adenovirus that is replication competent in one or more types of human neoplastic cells.

45. The viral composition of claim 44, wherein the adenovirus does not replicate in one or more non-neoplastic cells to the same extent that it replicates in neoplastic cells.

46. The viral composition of claim 44, wherein the adenovirus exhibits an upregulated expression of ADP relative to wild-type adenovirus.

47. A method of preparing a viral composition effective in inhibiting cancer comprising:
a) preparing a first solution comprising a viral vector comprising a therapeutic virus, in a concentration of about 10¹⁰ viral particles per 50 μL diluent;
b) preparing a second solution comprising a protamine molecule in a concentration of about 100 to 300 μg per 50 μL diluent;
c) mixing the first solution with the second solution in a ratio of about 1:1 to form a third solution; and
d) incubating the third solution for a time sufficient to complex the viral vector and the protamine molecule.

48. The method of claim 47, further comprising adding the viral composition to a pharmacologically acceptable diluent.

49. The method of claim 47, wherein the concentration is between about 1×10¹⁰ to about 5×10¹¹ viral particles.

50. The method of claim 47, wherein the viral vector is an adenoviral vector, a retroviral vector, a vaccinia viral vector, an adeno-associated viral vector, a polyoma viral vector, or a herpes viral vector.

51. The method of claim 47, wherein the viral vector is an adenoviral vector.

52. The method of claim 51, wherein the adenoviral vector lacks an E1b coding region.

53. The method of claim 47 or 51, wherein the viral vector comprises a nucleic acid encoding a tumor suppressor operably linked to a promoter.

54. The method of claim 53, wherein the tumor suppressor gene is p53, FHIT, MDA7, PTEN, or 3p21.3.

55. The method of claim 53, wherein the promoter is a CMV IE, dectin-1, dectin-2, human CD11c, F4/80, SM22 a MHC class II promoter, SV40, polyoma or adenovirus 2 promoter.

56. The method of claim 47, wherein the therapeutic adenovirus comprises an adenovirus that is replication competent in one or more types of human neoplastic cells.

57. The method of claim 56, wherein the adenovirus does not replicate in one or more non-neoplastic cells to the same extent that it replicates in neoplastic cells.

58. The method of claim 56, wherein the adenovirus exhibits an upregulated expression of ADP relative to wild-type adenovirus.
